SEQUENCE LISTING (26 sequences)

SEQ ID NO: 1 - protein, 11 aa, Artificial
  peptide fragment
SPSPAHIVSNK

SEQ ID NO: 2 - DNA, 29 bp, Artificial
  oligo for mutagenesis
ggccccatcaccggcacacattgtctcta

SEQ ID NO: 3 - DNA, 29 bp, Artificial
  oligo for mutagenesis
ggggcatccttgagggcttgtctactctg

SEQ ID NO: 4 - RNA, 21 nt, Artificial
  sense RNA for siRNA
cgacauacccagacugauauu

SEQ ID NO: 5 - RNA, 21 nt, Artificial
  antisense RNA for siRNA
uaucagucuggguaugucguu

SEQ ID NO: 6 - RNA, 21 nt, Artificial
  sense RNA for siRNA
gaccgaagcucuugguuacuu

SEQ ID NO: 7 - RNA, 21 nt, Artificial
  antisense RNA for siRNA
guaaccaagagcuucggucuu

SEQ ID NO: 8 - RNA, 21 nt, Artificial
  shRNA
acacacugguuuccauguaau

SEQ ID NO: 9 - RNA, 21 nt, Artificial
  shRNA
agagaaggaaccuugaugauu

SEQ ID NO: 10 - RNA, 21 nt, Artificial
  shRNA
agaaagauuucggugguaaau

SEQ ID NO: 11 - RNA, 21 nt, Artificial
  shRNA
ggaaucucauucgaugcauac

SEQ ID NO: 12 - DNA, 25 bp, Artificial
  oligo for mutagenesis
agggtcatggatgtcacaggggatg

SEQ ID NO: 13 - DNA, 27 bp, Artificial
  oligo for mutagenesis
aatagctgcaagctgtccggttttaac

SEQ ID NO: 14 - DNA, 35 bp, Artificial
  oligo for mutagenesis
atggccagtgattctccggctcgtagcctggatga

SEQ ID NO: 15 - DNA, 27 bp, Artificial
  oligo for mutagenesis
atcgatgggatcctgcaaaaagaacaa

SEQ ID NO: 16 - DNA, 25 bp, Artificial
  antisense morpholino oligonucleotide for Xenopus
gggagtcgctcgccatgtttccttt

SEQ ID NO: 17 - DNA, 25 bp, Artificial
  antisense morpholino oligonucleotide for Xenopus
ccccgttctttccaccttgcggctg

SEQ ID NO: 18 - DNA, 25 bp, Artificial
  control morpholino oligonucleotide for Xenopus
ggcagtggctccccatctttcgttt

SEQ ID NO: 19 - DNA, 25 bp, Artificial
  control morpholino oligonucleotide for Xenopus
ccgcgttgtttcgaccttccgcctg

SEQ ID NO: 20 - protein, 11 aa, Artificial
  peptide fragment
SPSPAHIVSNK

SEQ ID NO: 21 - protein, 11 aa, Artificial
  peptide fragment
SPSPAHIVSNK

SEQ ID NO: 22 - protein, 1360 aa, Homo sapiens
MASDSPARSLDEIDLSALRDPAGIFELVELVGNGTYGQVYKGRHVKTGQLAAIKVMDVTGDEEEEIKQEINMLKKYSHHRNIATYYGAFIKKNPPGMDDQLWLVMEFCGAGSVTDLIKNTKGNTLKEEWIAYICREILRGLSHLHQHKVIHRDIKGQNVLLTENAEVKLVDFGVSAQLDRTVGRRNTFIGTPYWMAPEVIACDENPDATYDFKSDLWSLGITAIEMAEGAPPLCDMHPMRALFLIPRNPAPRLKSKKWSKKFQSFIESCLVKNHSQRPATEQLMKHPFIRDQPNERQVRIQLKDHIDRTKKKRGEKDETEYEYSGSEEEEEENDSGEPSSILNLPGESTLRRDFLRLQLANKERSEALRRQQLEQQQRENEEHKRQLLAERQKRIEEQKEQRRRLEEQQRREKELRKQQEREQRRHYEEQMRREEERRRAEHEQEYIRRQLEEEQRQLEILQQQLLHEQALLLEYKRKQLEEQRQAERLQRQLKQERDYLVSLQHQRQEQRPVEKKPLYHYKEGMSPSEKPAWAKEVEERSRLNRQSSPAMPHKVANRISDPNLPPRSESFSISGVQPARTPPMLRPVDPQIPHLVAVKSQGPALTASQSVHEQPTKGLSGFQEALNVTSHRVEMPRQNSDPTSENPPLPTRIEKFDRSSWLRQEEDIPPKVPQRTTSISPALARKNSPGNGSALGPRLGSQPIRASNPDLRRTEPILESPLQRTSSGSSSSSSTPSSQPSSQGGSQPGSQAGSSERTRVRANSKSEGSPVLPHEPAKVKPEESRDITRPSRPASYKKAIDEDLTALAKELRELRIEETNRPMKKVTDYSSSSEESESSEEEEEDGESETHDGTVAVSDIPRLIPTGAPGSNEQYNVGMVGTHGLETSHADSFSGSISREGTLMIRETSGEKKRSGHSDSNGFAGHINLPDLVQQSHSPAGTPTEGLGRVSTHSQEMDSGTEYGMGSSTKASFTPFVDPRVYQTSPTDEDEEDEESSAAALFTSELLRQEQAKLNEARKISVVNVNPTNIRPHSDTPEIRKYKKRFNSEILCAALWGVNLLVGTENGLMLLDRSGQGKVYNLINRRRFQQMDVLEGLNVLVTISGKKNKLRVYYLSWLRNRILHNDPEVEKKQGWITVGDLEGCIHYKVVKYERIKFLVIALKNAVEIYAWAPKPYHKFMAFKSFADLQHKPLLVDLTVEEGQRLKVIFGSHTGFHVIDVDSGNSYDIYIPSHIQGNITPHAIVILPKTDGMEMLVCYEDEGVYVNTYGRITKDVVLQWGEMPTSVAYIHSNQIMGWGEKAIEIRSVETGHLDGVFMHKRAQRLKFLCERNDKVFFASVRSGGSSQVFFMTLNRNSMMNW

SEQ ID NO: 23 - protein, 485 aa, Xenopus
MASDSPARSLDEIDLSALRDPAGIFELVELVGNGTYGQVYKGRHVKTGQLAAIKVMDVTGDEEEEIKQEINMLKKYSHHRNIATYYGAFIKKNPPGMDDQLWLVMEFCGAGSVTDLIKNTKGNTLKEEWIAYICREILRGLSHLHQHKVIHRDIKGQNVLLTENAEVKLVDFGVSAQLDRTVGRRNTFIGTPYWMAPEVIACDENPDATYDFKSDLWSLGITAIEMAEGAPPLCDMHPMRALFLIPRNPAPRLKSKKWSKKFQSFIDSCLVKNHSQRPTTDQLMKHPFIRDQPNERQVRIQLKDHIDRTKKKRGEKDETEYEYSGSEEEEEDNDSGEPSSILNLPGESTLRRDFLRLQLANKERSEALRRQQLEQQQRENEEHKRQLLAERQKRIEEQKEQRRRLEEQQRKEKEMRKQQERDQRRRYEEQVRREEERRRAEHEKEYKRKQVEEQRQAERLQRQLQQERDYLVSLQQQQQQQRQQE

SEQ ID NO: 24 - DNA, 40 bp, Artificial
  forward primer
aatttcagggcgccatggcgagcgactccccggctcgaag

SEQ ID NO: 25 - DNA, 42 bp, Artificial
  reverse primer
attcgaaagcggccgctcatcctcgcttcttctttgttctat

SEQ ID NO: 26 - protein, 7 aa, Artificial
  substrate peptides
KYKTLRQ

(19) United States
(12) Patent Application Publication
(10) Pub. No.: US 2010/0137386 A1
(43) Pub. Date: Jun. 3, 2010

(54) TNIK INHIBITOR AND THE USE

(76) Inventors: Tesshi YAMADA, Tokyo (JP); Miki SHITASHIGE, Tokyo (JP); Koichi YOKOTA, Sakai-shi (JP); Masaaki SAWA, Ibaraki-shi (JP); Hideki MORIYAMA, Kobe-shi (JP)

Correspondence Address: WESTERMAN, HATTORI, DANIELS & ADRIAN, LLP, 1250 CONNECTICUT AVENUE, NW, SUITE 700, WASHINGTON, DC, 20036, US

(21) Appl. No.: 12/503,374

(22) Filed: Jul. 15, 2009

Related U.S. Application Data
(60) Provisional application No. 61/118,809, filed on Dec. 1, 2008.

(51) Int. Cl. A61K 31/426; C07D 277/56; A61P 35/00

(52) U.S. Cl. 514/371; 548/191

(57) ABSTRACT

The present invention relates to compositions and methods for the treatment of cancer patients with Traf2- and Nck-interacting kinase (TNIK) inhibitors. More particularly, the present invention relates to pharmaceutical compositions comprising TNIK inhibitor and a pharmaceutically acceptable carrier, and to methods for treating the TNIK inhibitor administered to cancer patients, especially to solid cancer patients such as colorectal cancer, pancreatic cancer, non-small cell lung cancer, prostate cancer or breast cancer.

And the present invention relates to a novel aminothiazole derivatives.

### FIELD OF THE INVENTION

The present invention relates to compositions and methods for the treatment of cancer patients with Traf2- and Nck-interacting kinase (TNIK) inhibitors. More particularly, the present invention relates to pharmaceutical compositions comprising TNIK inhibitor and a pharmaceutically acceptable carrier, and to methods for treating the TNIK inhibitor administered to cancer patients, especially to solid cancer patients such as colorectal cancer, pancreatic cancer, non-small cell lung cancer, prostate cancer or breast cancer.

And the present invention relates to a novel aminothiazole derivatives.

### BACKGROUND OF THE INVENTION

Wnt proteins are a large family of secreted glycoproteins that activate signal transduction pathways to control a wide variety of cellular processes such as determination of cell fate, proliferation, migration, and polarity. Wnt proteins are capable of signaling through several pathways, the best-characterized being the canonical pathway through β-catenin (Wnt/β-catenin signaling). Deregulation of Wnt/β-catenin signaling is frequently found in many human cancers like colorectal cancer (Näthke I., Nat Rev Cancer. 2006 December; 6(12):967-74), pancreatic cancer (Mimeault M and Batra S K., Gut. 2008 October; 57(10):1456-68), non-small cell lung cancer (Huang C L, Liu D, Ishikawa S, Nakashima T, Nakashima N, Yokomise H, Kadota K, Ueno M., Eur J Cancer. 2008 November; 44(17):2680-8), prostate cancer (Verras M, Sun Z., Cancer Lett. 2006 Jun. 8; 237(1):22-32), breast cancer (Huang C L, Liu D, Ishikawa S, Nakashima T, Nakashima N, Yokomise H, Kadota K, Ueno M., Eur J Cancer. 2008 November; 44(17):2680-8), and many others (Paul S and Dey A., Neoplasma. 2008; 55(3):165-76).

TNIK is known as one of STE20 family kinases that activates the c-Jun N-terminal kinase pathway and regulates the cytoskeleton (Fu C A, et al., J Biol Chem. 1999, 274:30729-37; Taira K, et al. J Biol Chem. 2004, 279:49488-96). Recently, TNIK was identified as one of 70 proteins immunoprecipitated commonly with anti-TCF4 in two colorectal cancer cell lines DLD1 and HCT-116 (Shitashige M, et al., Gastroenterology 2008, 134:1961-71).

More than 80% of colorectal cancers show mutation of the adenomatous polyposis coli (APC) gene¹, and half of the remainder do so in the CTNNB1 gene^{2,3}, resulting in accumulation of β-catenin and constitutive activation of Wnt signaling⁴⁻⁶. β-Catenin exerts its oncogenic activity by forming complexes with T-cell factor (TCF)/lymphoid enhancer factor (LEF) family DNA-binding proteins⁷⁻⁹ and by transactivating their target genes^{10,11} (Supplementary Fig. S1). TCF4 is a TCF/LEF family member commonly expressed in colorectal cancer cells¹² and implicated in colorectal carcinogenesis¹³. We previously identified TNIK as one of 70 proteins immunoprecipitated commonly with anti-TCF4 and anti-β-catenin antibodies in two colorectal cancer cell lines (DLD1 and HCT-116)¹⁴. DLD1 has a truncating mutation in the APC gene and loss of the other allele, and HCT-116 has a missense mutation in the CTNNB1 gene². TNIK was detected in the immunoprecipitates with anti-TCF4 or anti-β-catenin antibody, but not with control IgG. Conversely, β-catenin and TCF4 proteins were immunoprecipitated with anti-TNIK antibody (FIG. 1a), indicating that TCF4, β-catenin and TNIK proteins form a complex in colorectal cancer cells. Two-hybrid assay revealed that TNIK interacted with TCF4 through the amino acids 1-289 including the kinase domain (Supplementary Fig. S2a). The amino acids 100-216 of TCF4 were necessary for interaction with TNIK (Supplementary Fig. S2c).

TCF4 protein was phosphorylated by TNIK (WT, wild type) (FIG. 1b-e and Supplementary Fig. S3a-b), but not by the catalytically inactive mutant of TNIK with substitution (K54R) of the conserved lysine 54 residue in the ATP-binding pocket of the kinase domain¹⁵ (Supplementary FIG. 4). Tandem mass spectrometry (MS/MS) revealed that the serine 154 residue of TCF4 was phosphorylated by TNIK (WT) (Supplementary Fig. S3c-f). Consistently, substitution of the serine 154 residue by alanine (S154A) abolished the phosphorylation of TCF4 by TNIK (FIG. 1c). TCF4 was phosphorylated upon transfection of DLD1 cells with TNIK (WT), but not with TNIK (K54R) (FIG. 1d-e).

Auto-phosphorylation of TNIK¹⁵ seems to be necessary for its nuclear translocation and interaction with TCF4 (FIG. 1f-h and Supplementary Fig. S5). DLD1 and HCT-116 cells were transfected with TNIK (WT) or catalytically inactive TNIK (K54R) and analysed by immunoblotting (FIG. 1f) and immunofluorescence microscopy (Supplementary Fig. S5a). We found that TNIK (WT) induced the phosphorylation of its own serine 764 residue (TNIKpS764)¹⁶ (FIG. 1f, anti-TNIKpS764). The phosphorylated TNIK was incorporated into the nuclei, whereas the K54R substitution significantly inhibited the phosphorylation and nuclear translocation of TNIK (FIG. 1f, nuclear fraction, and Supplementary Fig. S5a) and reduced the amount of TNIK interacting with TCF4 (FIG. 1g). Endogenous TNIK protein was distributed along the filamentous cytoskeleton (Supplementary Fig. S5b), whereas phosphorylated TNIK (TNIKpS764) was detected mainly in the nuclei and co-localized with TCF4 (FIG. 1h). TNIKpS764 was detected in colorectal cancer cells, but not in untransformed HEK293 cells (Supplementary Fig. S6a).

The expression and localization of TNIK were examined in clinical specimens of colorectal cancer (Supplementary Fig. S6b-e). Although the overall expression level of TNIK protein did not differ significantly between cancer and normal mucosa (Supplementary Fig. S6b), the expression of phosphorylated TNIK (pS764) was increased in cancer cells compared to neighboring normal intestinal epithelial cells (Supplementary Fig. S6c-d). Nuclear TNIKpS764 was detected most predominantly in the invasive front of colorectal cancer (Supplementary Fig. S6e), where β-catenin was accumulated in the nucleus and cytoplasm (Supplementary Fig. S6f).

We then investigated the effects of TNIK on the transcriptional activity of the β-catenin and TCF4 complex (FIG. 2). HEK293 and HeLa cells have wild-type APC and CTNNB1 genes^{2,17}. Transient transfection of these cells with β-catenin stabilized by deletion of the N-terminal glycogen synthase kinase 3β (GSK3β)-phosphorylation site (β-cateninΔN134) increased the luciferase activity of the canonical TCF/LEF reporter (TOP-FLASH) in comparison with mock transfection, but did not increase the luciferase activity of the mutant reporter (FOP-FLASH) (FIG. 2a). Co-transfection with hemagglutinin (HA)-tagged wild-type TNIK (WT), but not with TNIK (K54R), further enhanced the β-catenin-evoked transcriptional activity (FIG. 2a) and colony formation by HEK293 and HeLa cells (FIG. 2b). TNIK did not significantly affect transcriptional activity or colony formation in the absence of β-cateninΔN134, indicating that the effects of TNIK are dependent upon activation of Wnt signaling. Transient transfection of DLD1 and HCT-116 cells with TNIK, but not with TNIK (K54R), also enhanced their TCF/LEF transcriptional activity and colony formation (Supplementary Fig. S7).

Conversely, knockdown of TNIK by short interfering RNA (siRNA) against TNIK (constructs 12 and 13) abolished the β-cateninΔN134-evoked TCF/LEF transcriptional activity of HEK293 and HeLa cells (Supplementary Fig. S8a). Knockdown of TNIK by short hairpin RNA (shRNA) against TNIK (constructs T1, T2 and T3) abolished the β-cateninΔN134-evoked colony formation, but did not significantly affect the proliferation of HEK293 and HeLa cells that were not co-transfected with β-cateninΔN134 (Supplementary Fig. S8b). Knockdown of TNIK suppressed the TCF/LEF transcriptional activity (FIG. 2c) and proliferation (FIG. 2d) of DLD1 and HCT-116 cells. The expression of known target genes of the β-catenin and TCF/LEF complexes, such as axis inhibitor-2 (AXIN2)¹⁸, c-myc (MYC)¹⁹, c-jun (JUN)²⁰ and matrilysin (MMP7)²¹, except for cyclin D1 (CCND1)²², was significantly reduced by transient transfection of siRNA against TNIK (Supplementary Fig. S9).

We next examined the effects of TNIK on the growth of human colon cancer cells in vivo (FIG. 3). HCT-116 cells were implanted in the flank of immunodeficient mice. One week after the inoculation, siRNA against TNIK (12 or 13) mixed with atelocollagen²³ was injected directly into the tumours (224.5±8.9 mm³ in size). Three days after the siRNA injection, some tumours were excised and the silencing of TNIK mRNA was confirmed by real-time PCR (FIG. 3b). The volume of xenografts was monitored for 18 days after siRNA injection (FIG. 3a). We found that the tumours regressed almost completely after a single injection of siRNA against TNIK (12 or 13). FIGS. 3c and 3d shows the appearance of representative mice and excised tumours. Tumours treated with siRNA against TNIK (12 or 13) were significantly smaller than those not treated (No treat), treated with only atelocollagen (Atelo only) or treated with control RNA (X or IX) (FIG. 3d). We observed similar regression of established tumours after a single injection of siRNA against TNIK in two other colorectal cancer cell lines, DLD1 and WiDr (Supplementary Figs. S10 and S11).

Finally, the functional involvement of TNIK in Wnt signaling was examined in Xenopus embryos (FIG. 4). There was a registry homologous to human TNIK in the Unigene Xenopus laevis database (named hypothetical protein LOC443633). The kinase domain (amino acids 25-289) was highly conserved (98.9%) between human and Xenopus (Supplementary Fig. S4b). Xenopus TNIK (XTNIK) was expressed maternally and the expression was maintained throughout the tadpole stages (Supplementary FIG. S12a). Ectopic activation of Wnt signaling in the ventral marginal zone is known to induce axis duplication²⁴. Co-injection with XTNIK (WT) mRNA enhanced X β-catenin-induced secondary axis formation, whereas catalytically inactive XTNIK (K54R) completely inhibited it (FIG. 4a). Embryos injected only with XTNIK (WT) or XTNIK (K54R) (without Xβ-catenin) developed normally (data not shown). In the animal cap assay (FIG. 4b), injection of X β-catenin induced the expression of known target genes of Wnt signaling: Siamois and Xnr3²⁵. Co-injection with XTNIK (WT) enhanced the expression of these genes, whereas XTNIK (K54R) shut down their expression.

Injection of XTNIK (K54R) mRNA into the dorsal blastomeres of 8-cell-stage embryos inhibited the initiation of gastrulation at stage 10 (FIG. 4c). Embryos injected dorsally with XTNIK (WT) showed a marked increase in the expression of Siamois and Xnr3, whereas XTNIK (K54R) decreased their expression (FIG. 4d). Embryos that received an injection of XTNIK (K54R) mRNA into the dorsal blastomeres at the 8-cell stage developed significant axis defects: complete loss of head and axis structures (FIG. 4e), typical phenotypes resulting from dorsal inhibition of Wnt signaling²⁶.

Antisense morpholino oligonucleotides (MOs) to XTNIK (MO1 and MO3) (Supplementary FIG. 12b) blocked secondary axis formation induced by X□-catenin when co-injected into the ventral marginal zone of 8-cell-stage embryos (Supplementary FIG. 12c-e). The blockage was abrogated by co-injection of HA-tagged XTNIK_{ORF (open reading frame)} mRNA [lacking the 5′UTR (5′-untranslated region) targeted by MO1 and MO3] (Supplementary FIG. 12d-e). Embryos injected dorsally with either XTNIK-MO failed to initiate gastrulation at stage 10 (Supplementary FIG. 13a-b) and developed into abnormal tadpoles with significantly reduced head and axis structures (Supplementary FIG. 14a-b). The defects caused by XTNIK-MOs were rescued by co-injection of XTNIK_{ORF} (Supplementary FIGS. 13 and 14). Embryos injected with control MOs with nucleotide mismatches (5mis-Control-1 and -3) did not show the effects observed in TNIK-MO1 and -MO3 (Supplementary FIGS. 12-14). The reduction of Siamois and Xnr3 expression by XTNIK-MOs was reversed by co-injection of XTNIK_{ORF} (Supplementary FIG. 13c).

Synthetic ATP-competitors of protein kinases have been incorporated successfully into oncological practice²⁷. For example, imatinib, which blocks the Bcr-Abl fusion kinase of chronic myeloid leukemia (CIVIL), is currently a first-line therapeutic drug for CML²⁸. The epidermal growth factor receptor (EGFR) tyrosine kinase inhibitors, gefitinib and erlotinib, have been used in the treatment of non-small cell lung cancer²⁹. Wnt signaling is a major force driving colorectal carcinogenesis. TNIK was activated in colorectal cancer, and colorectal cancer cells were highly dependent upon the expression and kinase activity of TNIK for proliferation. Our results indicate the feasibility of developing drugs that target TNIK.

### Methods Summary:

The methodological details of immunoprecipitation, immunoblotting, immunofluorescence microscopy, immunohistochemistry, mass spectrometry, two-hybrid assay, luciferase reporter assay, colony formation, real-time RT-PCR, axis formation assay and animal cap assay are available in FULL METHODS. Antibodies used in this study and their suppliers are listed in Supplementary Table S1. Mouse and Xenopus experiments were carried out according to the guidelines of the National Cancer Center Research Institute (Tokyo, Japan), which meet all the ethical requirements stipulated by Japanese law. The minimum number of mice necessary for obtaining reliable results were used and euthanatized. Patients submitted written informed consent authorizing the collection and use of their materials for research purposes. The experimental protocols were reviewed and approved by the institutional ethics and recombination safety committees.

### REFERENCES

1 Kinzler, K. W. & Vogelstein, B. Lessons from hereditary colorectal cancer. Cell 87, 159 (1996).
2 Morin, P. J. et al. Activation of β-catenin-Tcf signaling in colon cancer by mutations in β-catenin or APC. Science 275, 1787 (1997).
3 Sparks, A. B., Morin, P. J., Vogelstein, B. & Kinzler, K. W. Mutational analysis of the APC/β-catenin/Tcf pathway in colorectal cancer. Cancer Res 58, 1130 (1998).
4 Peifer, M. & Polakis, P. Wnt signaling in oncogenesis and embryogenesis—a look outside the nucleus. Science 287, 1606 (2000).
5 Polakis, P. Wnt signaling and cancer. Genes Dev 14, 1837 (2000).
6 Clevers, H. Wnt breakers in colon cancer. Cancer Cell 5, 5 (2004).
7 Behrens, J. et al. Functional interaction of β-catenin with the transcription factor LEF-1. Nature 382, 638 (1996).
8 Huber, O. et al. Nuclear localization of β-catenin by interaction with transcription factor LEF-1. Mech Dev 59, 3 (1996).
9 van de Wetering, M. et al. Armadillo coactivates transcription driven by the product of the Drosophila segment polarity gene dTCF. Cell 88, 789 (1997).
10 van Es, J. H., Barker, N. & Clevers, H. You Wnt some, you lose some: oncogenes in the Wnt signaling pathway. Curr Opin Genet Dev 13, 28 (2003).
11 Wong, N. A. & Pignatelli, M. βCatenin—a linchpin in colorectal carcinogenesis?Am J Pathol 160, 389 (2002).
12 Korinek, V. et al. Constitutive transcriptional activation by a β-catenin-Tcf complex in APC−/− colon carcinoma. Science 275, 1784 (1997).
13 Korinek, V. et al. Depletion of epithelial stem-cell compartments in the small intestine of mice lacking Tcf-4. Nat Genet 19, 379 (1998).
14 Shitashige, M. et al. Regulation of Wnt signaling by the nuclear pore complex. Gastroenterology 134, 1961 (2008).
15 Fu, C. A. et al. TNIK, a novel member of the germinal center kinase family that activates the c-Jun N-terminal kinase pathway and regulates the cytoskeleton. J Biol Chem 274, 30729 (1999).
16 Wissing, J. et al. Proteomics analysis of protein kinases by target class-selective prefractionation and tandem mass spectrometry. Mol Cell Proteomics 6, 537 (2007).
17 Naishiro, Y. et al. Morphological and transcriptional responses of untransformed intestinal epithelial cells to an oncogenic β-catenin protein. Oncogene 24, 3141 (2005).
18 Yan, D. et al. Elevated expression of axin2 and hnkd mRNA provides evidence that Wnt/β-catenin signaling is activated in human colon tumors. Proc Natl Acad Sci USA 98, 14973 (2001).
19 He, T. C. et al. Identification of c-MYC as a target of the APC pathway. Science 281, 1509 (1998).
20 Mann, B. et al. Target genes of β-catenin-T cell-factor/lymphoid-enhancer-factor signaling in human colorectal carcinomas. Proc Natl Acad Sci USA 96, 1603 (1999).
21 Crawford, H. C. et al. The metalloproteinase matrilysin is a target of β-catenin transactivation in intestinal tumors. Oncogene 18, 2883 (1999).
22 Tetsu, O. & McCormick, F. β-Catenin regulates expression of cyclin D1 in colon carcinoma cells. Nature 398, 422 (1999).
23 Takeshita, F. et al. Efficient delivery of small interfering RNA to bone-metastatic tumors by using atelocollagen in vivo. Proc Natl Acad Sci USA 102, 12177 (2005).
24 Funayama, N., Fagotto, F., McCrea, P. & Gumbiner, B. M. Embryonic axis induction by the armadillo repeat domain of β-catenin: evidence for intracellular signaling. J Cell Biol 128, 959 (1995).
25 McKendry, R., Hsu, S. C., Harland, R. M. & Grosschedl, R. LEF-1/TCF proteins mediate wnt-inducible transcription from the Xenopus nodal-related 3 promoter. Dev Biol 192, 420 (1997).
26 Heasman, J., Kofron, M. & Wylie, C. β-Catenin signaling activity dissected in the early Xenopus embryo: a novel antisense approach. Dev Biol 222, 124 (2000).
27 Hunter, T. Treatment for chronic myelogenous leukemia: the long road to imatinib. J Clin Invest 117, 2036 (2007).
28 Druker, B. J. et al. Five-year follow-up of patients receiving imatinib for chronic myeloid leukemia. N Engl J Med 355, 2408 (2006).
29 Fong, T., Morgensztern, D. & Govindan, R. EGFR inhibitors as first-line therapy in advanced non-small cell lung cancer. J Thorac Oncol 3, 303 (2008).

### FIGURE LEGENDS

FIG. 1|Phosphorylation of TCF4 by TNIK.

a, Total lysates (Total) and immunoprecipitates (IP) with anti-TCF4, anti-β-catenin (β-cat), anti-TNIK antibody or control IgG, of DLD1 and HCT-116 cells, were blotted with the indicated antibodies.

b, c, Glutathione S-transferase (GST), GST-TCF4 (WT) and GST-TCF4 (S154A) proteins were incubated with the in-vitro translation product of pCIneoHA-TNIK-WT, TNIK-K54R or empty plasmid (Cont) at 30° C. for 30 minutes in the presence of 0.1 mM of ATP, immunoprecipitated with anti-GST antibody and blotted with anti-phosphoserine (pSer) and anti-GST antibodies. The blots at the bottom (anti-HA) indicate the comparable production of TNIK-WT and TNIK-K54R proteins.

d, e, DLD1 cells were transfected with pFLAG-TCF4 and pCIneoHA-TNIK-WT, TNIK-K54R or empty plasmid (Cont). Immunoprecipitates with anti-TCF4 antibody or control IgG were blotted with the indicated antibodies. The comparable expression of TCF4, TNIK and β-actin (loading control) proteins was confirmed by immunoblotting.

f, DLD1 or HCT-116 cells were transfected with 1 or 2 μg of pCIneoHA-TNIK-WT, 1 or 2 μg of pCIneoHA-TNIK-K54R or 2 μg of empty plasmid (Control). The total amount of DNA used for transfection was kept constant by adding empty plasmid DNA. Total cell lysates and nuclear fraction proteins were blotted with the indicated antibodies.

g, Total lysates of cells transfected as described in (d, e) were immunoprecipitated and blotted with the indicated antibodies.

h, Localization of endogenous TNIKpS764 and TCF4 proteins in colorectal cancer cells.

FIG. 2|Enhancement of TCF/LEF transcriptional activity by TNIK.

a, b, HEK293 or HeLa cells were co-transfected with pFLAG-β-cateninΔN134 (+) or empty plasmid (pFLAG-CMV4) (−) and pCIneoHA-TNIK-WT, -TNIK-K54R or empty plasmid (Cont).

c, DLD1 or HCT-116 cells were co-transfected in triplicate with control RNA (X or IX) or siRNA against TNIK (12 and 13).

d, DLD1 or HCT-116 cells were transfected with shRNA constructs, pGeneClip-TNIK1, -TNIK2, -TNIK3 or -control.

a-d, Luciferase activity and colony formation were assessed as described in FULL METHODS. The expression levels of β-catenin, TNIK and β-actin (loading control) proteins were analysed by immunoblotting.

FIG. 3|Inhibition of colorectal cancer growth by siRNA against TNIK.

a, HCT-116 cells were inoculated into BALB/c nu/nu nude mice subcutaneously. The developed tumours were not treated, or treated with only atelocollagen, control RNA (X or IX) or siRNA against TNIK (12 or 13). The volume of tumours (n=8 per group) was monitored as indicated. #, P<0.001 (Mann-Whitney U-test); Bars, SE; N. S., not significant.

b, mRNA expression of TNIK in tumours injected with siRNA (n=3 per group).

c, d, Representative appearance of mice and excised tumours 18 days after the injection of siRNA. Columns indicate the average weight of excised tumours (n=8 per group). $, P<0.005 (Mann-Whitney U-test); Bars, SE.

FIG. 4|Regulation of Wnt signaling by TNIK in Xenopus embryos.

mRNA for Xβ-catenin (25 pg), nuclear β-galactosidase (n-βgal) (500 pg), XTNIK-WT-Myc (500 pg) or XTNIK-K54R-Myc (500 pg) was injected in the indicated combinations into the ventral marginal zone of 8-cell-stage embryos (a), animal poles of 4-cell-stage embryos (b) or dorsal marginal zone of 8-cell-stage embryos (c-e).

a, Representative appearance of tadpoles and the ratios of tadpoles with secondary axis formation. “Complete” indicates axis formation with head to trunk structures. “Partial” indicates axis formation without heads. Secondary axes with or without cement glands were counted as “Complete” or “Partial”, respectively. The expression levels of Xβ-catenin, Myc-tagged XTNIK and β-actin (loading control) proteins at stage 15 were determined by immunoblotting.

b, mRNA expression of Siamois and Xnr3 determined by real-time PCR. Twenty animal caps per group were dissected at stage 9, cultured for 30 minutes, and then harvested for RNA isolation. AC, animal cap; RT, reverse transcription.

c, Representative embryos at stage 10+ with the vegetal poles up and the dorsal sides left. The ratios of embryos with dorsal blastopore lip formation are shown in columns.

d, Embryos were harvested at stage 9 and their relative mRNA expression of Siamois and Xnr3 was determined by real-time PCR.

e, The ratios of tadpoles with eyes at stage 35 are shown in columns.

### Legends for Supplementary Figures

Supplementary FIG. S1|Activation of Wnt signaling in colorectal cancer.

### (Background and Main Finding)

a, The APC protein forms a complex with β-catenin, axin, GSK3β and others. The protein complex is necessary for the phosphorylation of β-catenin by GSK3β. Phosphorylated β-catenin is rapidly degraded through the ubiquitin-proteasome pathway^{1,2}.

b, In colorectal cancer cells the protein complex cannot be assembled due to a truncating mutation of the APC gene³, resulting in accumulation of β-catenin and constitutive activation of Wnt signaling^{1, 2, 4}. β-Catenin exerts its oncogenic activity by activating a transcription factor, TCF4⁵⁻⁷. Because restoration of the loss of function resulting from mutation of the APC gene is not a realistic approach, we have been searching for molecules involved in the Wnt signaling pathway downstream of APC, especially in the nucleus⁸, that might be potential drug targets. In this study we report the identification of TNIK as the activating kinase of the TCF4 and β-catenin transcriptional complex.

Supplementary Figure S2|Domains necessary for the interaction between TNIK and TCF4.

a, b, HEK293 cells were co-transfected with the pAct vector carrying the entire coding sequence of TCF4 cDNA (pAct-TCF4—WT, ▪) or an empty vector (pAct-Control, □), the pBind plasmid carrying one of the serial deletion mutants of TNIK (b) or an empty pBind vector (Control) and pG5luc plasmid.

c, d, HEK293 cells were co-transfected with the pBind vector carrying amino acids 1-289 of TNIK [pBind-TNI K(1-289), ≡] or an empty vector [pBind-Control, □] and the pAct plasmid carrying one of the truncated forms of TCF4 (d) or an empty pAct vector (Control) and pG5luc plasmid.

a, c, The expression of constructs was confirmed by immunoblotting with anti-GAL4 (Bind) and VP16 (Act) antibodies. Forty-eight hours after transfection, luciferase activity was measured using Renilla reniformis luciferase activity as an internal control. Bars, SD; NLS, nuclear localization signal; CNH, citron homology.

Supplementary Figure S3|Phosphorylation site of TCF4.

a, b, HEK293 cells were transfected with a pCIneoHA-TNIK-WT, -TNIK-K54R or empty plasmid (Cont). Twenty four hours later, the comparable expression of TNIK proteins was confirmed by blotting with anti-HA and anti-β-actin antibodies. The immunoprecipitates (IP) with anti-HA antibody were incubated with GST-TCF4 recombinant protein at 30° C. for 30 minutes in the presence of 0.1 mM ATP and analysed by blotting directly with anti-pSer and anti-GST antibodies (a) or by immunoprecipitation and blotting with the indicated antibodies (b).

c-f, MS/MS spectra of the GST-TCF4 recombinant protein incubated with Control (c), TNIK-K54R (d) or TNIK-WT (e) protein. 168.0- and 608.8-m/z MS peaks corresponding to phosphoserine (fragment ion, b1) and pSPSPAHIVSNK ([M+2H]²⁺), respectively, were detected only in TNIK-WT (e) with high confidence scores (f). N.D., not detected.

Supplementary Figure S4|Human and Xenopus TNIK proteins.

a. Domain structure of human TNIK. CNH, citron homology.

b, Alignment of Xenopus laevis hypothetical protein LOC443633 (BOTTOM) with the human TNIK amino acid sequence (TOP).

Supplementary Figure S5|Phosphorylation-dependent nuclear localization of TNIK.

a, DLD1 cells were transfected with pCIneoHA-TNIK-WT (WT) or -TNIK-K54R (K54R). Twenty four hours after transfection, the localizations of transfected TNIK (GREEN) and endogenous TCF4 (RED) proteins were visualized by immunofluorescence staining with anti-HA rabbit polyclonal (GREEN) and anti-TCF4 mouse monoclonal (RED) antibodies.

b, Immunofluorescence microscopy analysis of endogenous TNIK (GREEN) and TCF4 (RED) proteins.

Supplementary Figure S6|Detection of phosphorylated TNIK in colorectal cancer.

a, Immunoblotting of total lysates extracted from HEK293, DLD1 or HCT-116 cells with the indicated antibodies.

b-f, Immunoperoxidase staining of the TNIK (b), phosphorylated TNIK (TNIKpS764) (c-e) and β-catenin (f) proteins in clinical samples of colorectal cancer. “N” and “C” indicate normal mucosa and cancer, respectively. The area in the orange square in (c) has been enlarged and is shown in (d).

Supplementary Figure S7|Regulation of TCF/LEF transcriptional activity by TNIK.

a, DLD1 or HCT-116 cells were co-transfected in triplicate with pCIneoHA-TNIK-WT (WT), -TNIK-K54R (K54R) or empty plasmid (pCIneoHA) (Cont) and one of the reporter plasmids (TOP-FLASH or FOP-FLASH). Twenty-four hours after transfection, luciferase activities were measured using Renilla reniformis luciferase activity as an internal control. Bars, SD.

b, Colony formation by DLD1 or HCT-116 cells transfected with pCIneoHA-TNIK-WT (WT), -TNIK-K54R (K54R) or empty plasmid (pCIneoHA) (Cont). Transfectants were cultured in the presence of G418 for 8 days and then stained.

Supplementary Figure S8|Suppression of TCF/LEF transcriptional activity by knockdown of TNIK.

a, HEK293 or HeLa cells were co-transfected in triplicate with one of the reporter plasmids (TOP-FLASH or FOP-FLASH), pFLAG-β-cateninΔN134 (+) or its relevant empty plasmid (pFLAG-CMV4) (−) and control siRNA (X or IX) or siRNA against TNIK (12 or 13). Forty-eight hours after transfection luciferase activities were measured. Bars, SD.

b, Colony formation by HEK293 or HeLa cells co-transfected with pFLAG-β-cateninΔN134 (+) or its relevant empty plasmid (pFLAG-CMV4) (−) and pGeneClip-TNIK1 (T1), -TNIK2 (T2), TNIK3 (T3) or -Control (C).

a, b, The expression levels of β-cateninΔN134, TNIK and β-actin (loading control) proteins were analysed by immunoblotting.

Supplementary Figure S9|Regulation of TCF/LEF target gene expression by TNIK. DLD1 or HCT-116 cells were transfected with siRNA against TNIK (12 or 13) or control RNA (X or IX). Forty eight hours after transfection, the relative expression levels of genes encoding axis inhibitor-2 (AXIN2), c-myc (MYC), c-jun (JUN) and matrilysin (MMP7) and cyclin D1 (CCND1) were quantified by real-time RT-PCR and expressed as ratios (MET) relative to the controls (X).

Supplementary Figures S10 and S11|Growth inhibition and regression of colorectal tumours by siRNA against TNIK.

DLD1 (S10) or WiDr (S11) cells were inoculated into the flanks of BALB/c nu/nu nude mice on day 0. Developed tumours (DLD1, 64.0±1.9 mm³; WiDr, 95.9±1.6 mm³) were not treated or treated with only atelocollagen, control RNA (X or IX) or siRNA against TNIK (12 or 13) on day 7.

a, Volume of tumours (n=8 per group) measured on days 7, 9, 13, 16, 19, 22 and 25.

*, P<0.0001 (Mann-Whitney U-test); Bars, SE; N.S., not significant.

b, mRNA expression of TNIK in tumours (n=3 per group) determined by real-time PCR 3 days after siRNA injection.

c, d, Representative appearance of mice (c) and excised tumours (d) and the weight of excised tumours (n=8 per group) (d) on day 25 (18 days after the injection of siRNA). $, P<0.005 (Mann-Whitney U-test); Bars, SE.

Supplementary Figure S12|Translational blockage of XTNIK inhibits Wnt signaling in Xenopus embryos

a, Relative expression of XTNIK was quantified by real-time PCR and expressed as a ratio (ΔΔCT) relative to the expression of the ornithine decarboxylase (odc) gene. Embryos at the 4-cell stage, stage 9 and stage 10.5 were divided into the dorsal (ORANGE) and ventral (BLUE) sides.

b, Protein expression of embryos co-injected with XTNIK-WT-Myc or XTNIK_{ORF}-HA mRNA and 5mis-Control-1, -Control-3, XTNIK-MO1 or -MO3 morpholino oligonucleotide (MO). 5mis-Control-1 and -Control-3 contain 5 nucleotide substitutions in the sequences of XTNIK-MO1 and -MO3, respectively, and did not suppress the translation of XTNIK-WT-Myc (blots with anti-Myc). XTNIK_{ORF}-HA lacks the 5′UTR (5′-untranslated region) that is targeted by antisense MOs XTNIK-MO1 and -MO3. The translation of XTNIK-WT-Myc (blots with anti-Myc), but not that of XTNIK_{ORF}-HA (blots with anti-HA), was inhibited by XTNIK-MO1 and -MO3.

c-e, 5-mis-Control-1 (40 ng), -Control-3 (20 ng), XTNIK-MO1 (40 ng), or -MO3 (20 ng), XTNIK_{ORF}-HA (500 pg) mRNA and X□-catenin mRNA (50 pg) were co-injected in the indicated combinations into the ventral marginal zone of 8-cell-stage embryos.

c, Injected embryos were harvested at stage 10 and examined by immunoblotting.

d, e, Representative appearance of tadpoles and the ratios of tadpoles with secondary axis formation. “Complete” indicates axis formation with head to trunk structures. “Partial” indicates axis formation without heads. Secondary axes with or without cement glands were counted as “Complete” or “Partial”, respectively.

Supplementary Figure S13|Translational blockage of XTNIK inhibits Wnt signaling in Xenopus embryos

5-mis-Control-1 (40 ng), -Control-3 (40 ng), XTNIK-MO1 (40 ng), or -MO3 (40 ng) and XTNIK_{ORF}-HA (500 pg) mRNA were co-injected in the indicated combinations into the dorsal marginal zone of 8-cell-stage embryos.

a, b, Representative embryos at stage 10+ with the vegetal poles up and the dorsal sides left. The ratios of embryos with dorsal blastopore lip formation are shown in the columns.

c, Expression of Siamois and Xnr3 of stage 9 embryos determined by real-time PCR.

Supplementary Figure S14|Translational blockage of XTNIK inhibits Wnt signaling in Xenopus embryos

5-mis-Control-1 (40 ng), -Control-3 (40 ng), XTNIK-MO1 (40 ng), or -MO3 (40 ng) and XTNIK_{ORF}-HA (500 pg) mRNA were co-injected in the indicated combinations into the dorsal marginal zone of 8-cell-stage embryos.

a, b, Representative appearance of tadpoles and the ratios of tadpoles with eyes.

### REFERENCES

1. Peifer, M. & Polakis, P. Wnt signaling in oncogenesis and embryogenesis—a look outside the nucleus. Science 287, 1606-1609 (2000).
2. Polakis, P. Wnt signaling and cancer. Genes Dev 14, 1837-1851 (2000).
3. Kinzler, K. W. & Vogelstein, B. Lessons from hereditary colorectal cancer. Cell 87, 159-170 (1996).
4. Clevers, H. Wnt breakers in colon cancer. Cancer Cell 5, 5-6 (2004).
5. van de Wetering, M. et al. Armadillo coactivates transcription driven by the product of the Drosophila segment polarity gene dTCF. Cell 88, 789-799 (1997).
6. Huber, O. et al. Nuclear localization of β-catenin by interaction with transcription factor LEF-1. Mech Dev 59, 3-10 (1996).
7. Behrens, J. et al. Functional interaction of β-catenin with the transcription factor LEF-1. Nature 382, 638-642 (1996).
8. Shitashige, M., Hirohashi, S. & Yamada, T. Wnt signaling inside the nucleus. Cancer Sci 99, 631-637 (2008).

### Full Methods:

### Cell Lines:

The human embryonic kidney cell line HEK293 and the human colorectal cancer cell line DLD1 were obtained from the Health Science Research Resources Bank (Osaka, Japan). The human cervical cancer cell line HeLa was obtained from the Riken Cell Bank (Tsukuba, Japan). Human colorectal cancer cell lines HCT-116 and WiDr were purchased from the American Type Culture Collection (Rockville, Md.).

### Antibodies:

Antibodies used in this study and their suppliers are listed in Supplementary Table S1.

### Immunoprecipitation:

Total cell lysates were prepared as described previously¹. The lysates were incubated at 4° C. overnight with the indicated antibody or relevant control IgG and precipitated with Dynabeads protein G (Dynal Biotech, Oslo, Norway).

### Immunoblot Analysis:

Protein samples were fractionated by SDS-PAGE and blotted onto Immobilon-P membranes (Millipore, Billerica, Mass.). After incubation with the primary antibodies at 4° C. overnight, the blots were detected with the relevant horseradish peroxidase-conjugated anti-mouse or anti-rabbit IgG antibody and ECL Western blotting detection reagents (GE Healthcare, Giles, UK). Blot intensity was quantified using a LAS-3000 scanner and Science Lab 2003 software (Fuji Film, Tokyo, Japan)¹.

### Mass Spectrometry (MS):

Protein bands in SDS-PAGE gels were visualized by Coomassie blue staining and digested using modified trypsin (Promega) as described previously. The tryptic peptides were concentrated and desalted with a 500-μm i.d.×1 mm HiQ sil C18-3 trapping column (KYA Technologies, Tokyo, Japan). The peptides were then fractionated with a 0-80% acetonitrile gradient (200 mL/minute for 1 hour) using a 150-μm i.d.×5 cm C18W-3 separation column (KYA) and analysed with a Q-Star Pulsar-i mass spectrometer equipped with a nanospray ionization source (Applied Biosystems, Foster City, Calif.). Reliability of protein identification was estimated by calculating the Confidence Value using ProID software (Applied Biosystems)¹.

### Plasmids:

Human TNIK (Traf2- and Nck-interacting kinase) expression constructs [pCIneoHA-TNIK and its mutant (K54R)]³ were kindly provided by Drs. M. Umikawa and K. Kariya (University of the Ryukyus, Nishihara-cho, Japan). cDNA sequences encoding different parts of TNIK protein were subcloned into pBind (Promega, Madison, Wis.). Human TCF4 (T-cell factor-4) (splice form E) and β-catenin cDNA lacking the 134-amino-acid sequence in its NH₂-terminus were subcloned into pFLAG-CMV4 (Sigma-Aldrich, St. Louis, Mo.)⁴. Full-length human TCF4 cDNA and its truncated forms were subcloned into pAct (Promega). The mutant form of TCF4, designated as TCF4S154A, was constructed using a QuikChange mutagenesis kit (Stratagene, La Jolla, Calif.) with oligos GGCCCCATCACCGGCACACATTGTCTCTA and GGGGCATCCTTGAGGGCTTGTCTACTCTG to change the serine (S) 154 residue to alanine (A). Full-length human TCF4 and TCF4S154A cDNAs were subcloned into pEU-E01-MCS (CellFree Science, Matsuyama, Japan).

### Mammalian Two-Hybrid Assay:

Physical interactions between the TNIK and TCF4 proteins were assessed using the CheckMate Mammalian Two-hybrid system (Promega), according to instructions provided by the supplier. HEK293 cells were co-transfected in triplicate with pBind, pAct and pG5luc (Promega) plasmids using the Lipofectamine 2000 reagent (Invitrogen, Carlsbad, Calif.)^{2,5}.

### Nuclear Protein Extraction:

Nuclear proteins were extracted using NE-PER Nuclear and Cytoplasmic Extraction Reagents (Pierce, Rockford, Ill.).

### Recombinant Protein Production:

Glutathione S-transferase (GST)-fusion proteins were synthesized using the ENDEXT Wheat Germ Expression Kit (CellFree Sciences, Matsuyama, Japan). HA (hemagglutinin)-tagged recombinant TNIK proteins were synthesized using rabbit reticulocyte lysate (TnT T7 Quick Coupled Transcription/Translation System) (Promega, Madison, Wis.).

### Immunofluorescence Cytochemistry:

Cells cultured on glass coverslips (Asahi Technoglass, Tokyo, Japan) were fixed with 4% paraformaldehyde at room temperature for 10 minutes and permeabilized with 0.2% Triton X-100. After blocking with 10% normal swine serum (Vector Laboratories, Burlingame, Calif.), the cells were incubated with primary antibodies at 4° C. overnight and subsequently with Alexa fluor-594 anti-mouse and Alexa fluor-488 anti-rabbit antibodies (Invitrogen). The specimens were examined with a laser scanning microscope (LSM5 PASCAL; Carl Zeiss, Jena, Germany)⁶.

### RNA Interference:

Two small interfering RNAs (siRNAs), TNIK-J-004542-12 (sense: 5′-CGACAUACCCAGACUGAUAUU-3′; antisense: 5′-PUAUCAGUCUGGGUAUGUCGUU-3′) and TNIK-J-004542-13 (sense: 5′-GACCGAAGCUCUUGGUUACUU-3′; antisense: 5′-PGUAACCAAGAGCUUCGGUCUU-3′), were synthesized and annealed by Dharmacon (Chicago, Ill.). Two control RNAs (X and IX) were purchased from Dharmacon.

The SureSilencing short hairpin (sh)RNA plasmid for human TNIK (T1, ACACACTGGTTTCCATGTAAT; T2, AGAGAAGGAACCTTGATGATT; T3, AGAAAGATTTCGGTGGTAAAT) and negative control (C, GGAATCTCATTCGATGCATAC) were purchased from SuperArray Bioscience (Frederick, Md.).

### Luciferase Reporter Assay:

A pair of luciferase reporter constructs, TOP-FLASH and FOP-FLASH (Upstate, Charlottesville, Va.), were used to evaluate TCF/LEF (lymphoid enhancer factor) transcriptional activity. Cells were transiently transfected in triplicate with one of the luciferase reporters and phRL-TK (Promega). Luciferase activity was measured with the Dual-luciferase Reporter Assay system (Promega) using Renilla reniformis luciferase activity as an internal control⁴.

### Colony Formation Assay:

Twenty four hours after transfection, 750, 400, 300 and 1000 μg/ml G418 (Geneticin, Invitrogen) was added to the culture media of HEK293, HeLa, DLD1 and HCT-116 cells, respectively. Cells were stained with Giemsa solution (Wako, Osaka, Japan) after selection for 8 days¹.

### Real-Time RT-PCR:

Total RNA was prepared with an RNeasy Mini Kit (Qiagen, Valencia, Calif.). DNase-1-treated RNA was random-primed and reverse-transcribed using SuperScript II reverse transcriptase (Invitrogen). The TaqMan universal PCR master mix and pre-designed TaqMan Gene Expression probe and primer sets were purchased from Applied Biosystems. Amplification data measured as an increase in reporter fluorescence were collected using the PRISM 7000 Sequence Detection system (Applied Biosystems). mRNA expression level relative to the internal control [β-actin (ACTB) for human or ornithine decarboxylase (odc) for Xenopus] was calculated by the comparative threshold cycle (C_{T}) method⁵.

Mouse Experiments: 5×10⁶ HCT-116, DLD1 or WiDr cells suspended in 0.1 ml of PBS were subcutaneously inoculated into the flanks of 5-week-old female BALB/c nu/nu nude mice (SLC, Tokyo, Japan). One week later the developed tumours were treated with siRNA together with atelocollagen (AteloGene; KOKEN, Tokyo, Japan)⁷. The final concentration of siRNA was 30 μM and that of atelocollagen was 0.5%. A 0.2-ml volume of siRNA solution was injected directly into each tumour. Tumour volume was determined as V=A×B²π/6, where A and B represent the largest and smallest dimensions⁸.

### Immunohistochemistry:

Fifty cases of sporadic colorectal cancer were selected from the pathology archive panel of the National Cancer Center Hospital (Tokyo, Japan). Immunoperoxidase staining was performed using the avidin-biotin complex method as described previously⁹.

### Xenopus Experiments:

Preparation of Xenopus laevis embryos and microinjection have been described previously¹⁰. Eggs were fertilized in vitro and dejellied with 1% sodium thioglycolate solution. Embryos were staged according to Niewkoop and Faber¹¹. Capped mRNA was synthesized by in-vitro transcription (mMESSAGE mMACHINE kit; Ambion, Austin, Tex.). Injection was performed in Steinberg's solution containing 5% Ficoll.

pCS2-FLAG-Xenopus β-catenin (Xβ-catenin)¹² was kindly provided by Dr. S. Sokol (Mount Sinai School of Medicine, New York, N.Y.) and pCS2-nβ-Gal^{13,14} was kindly provided by Drs. D. Turner, R. Rupp and J. Lee (Fred Hutchinson Cancer Research Center, Seattle, Wash.). pCS2+-Myc and pCS2+-HA were kindly provided by Dr. M. Taira (University of Tokyo, Tokyo, Japan).

The open reading frame (ORF) and 5′-untranslated region (5′UTR) of LOC443633 (XTNIK), which are recognized by antisense morpholino oligonucleotide (MO) XTNIK-MO1 or MO3, were PCR-amplified from the Xenopus laevis IMAGE cDNA clone MXL1736-98358477 (Open Biosystems, Huntsville, Ala.) and subcloned into pCS2+-Myc (pCS-XTNIK-WT-Myc). The mutant form of pCS-XTNIK-WT-Myc (pCS-XTNIK-K54R-Myc) was constructed by mutagenesis with oligos AGGGTCATGGATGTCACAGGGGATG and AATAGCTGCAAGCTGTCCGGTTTTAAC to change the lysine (K) 54 residue to arginine (R).

The ORF sequence of XTNIK, which is not recognized by XTNIK-MO1 or MO3, was constructed by mutagenesis with oligos ATGGCcAGtGAtTCtCCGGCTCGTAGCCTGGATGA (small letters indicate modifications) and ATCGATGGGATCCTGCAAAAAGAACAA (pC52-XTN1K_{ORF}-HA).

### Antisense Morpholino Oligonucleotides (MOs):

The antisense MOs for Xenopus TNIK (XTNIK-MO1 and -MO3) and the corresponding control MOs [carrying 5 nucleotide substitutions within XTNIK-MO1 and -MO3 sequences (5mis-Controls-1 and -3)] were obtained from Gene Tools (Philomath, Oreg.). A database search confirmed the absence of a significant homologous sequence to the complements of XTNIK-MO1 and -MO3 in Xenopus laevis. The sequences of MOs used in this study were: XTNIK-MO1 (5′-GGGAGTCGCTCGCCATGTTTCCTTT-3′), XTNIK-MO3 (5′-CCCCGTTCTTTCCACCTTGCGGCTG-3′), 5mis-Control-1 (5′-GGCAGTGGCTCCCCATCTTTCGTTT-3′) and 5 mis-Control-3 (5′-CCGCGTTGTTTCGACCTTCCGCCTG-3′).

### REFERENCES

1 Shitashige, M. et al. Regulation of Wnt signaling by the nuclear pore complex. Gastroenterology 134, 1961 (2008).
2 Sato, S. et al. β-Catenin interacts with the FUS proto-oncogene product and regulates pre-mRNA splicing. Gastroenterology 129, 1225 (2005).
3 Taira, K. et al. The Traf2- and Nck-interacting kinase as a putative effector of Rap2 to regulate actin cytoskeleton. J Biot Chem 279, 49488 (2004).
4 Idogawa, M. et al. Poly(ADP-ribose) polymerase-1 is a component of the oncogenic T-cell factor-4/β-catenin complex. Gastroenterology 128, 1919 (2005).
5 Huang, L. et al. Functional interaction of DNA topoisomerase IIα with the β-catenin and T-cell factor-4 complex. Gastroenterology 133, 1569 (2007).
6 Idogawa, M. et al. Ku70 and poly(ADP-ribose) polymerase-1 competitively regulate β-catenin and T-cell factor-4-mediated gene transactivation: possible linkage of DNA damage recognition and Wnt signaling. Cancer Res 67, 911 (2007).
7 Takeshita, F. et al. Efficient delivery of small interfering RNA to bone-metastatic tumors by using atelocollagen in vivo. Proc Natl Acad Sci USA 102, 12177 (2005).
8 Bergers, G et al. Effects of angiogenesis inhibitors on multistage carcinogenesis in mice. Science 284, 808 (1999).
9 Shitashige, M. et al. Involvement of splicing factor-1 in β-catenin/T-cell factor-4-mediated gene transactivation and pre-mRNA splicing. Gastroenterology 132, 1039 (2007).
10 Satow, R., Chan, T. C. & Asashima, M. Molecular cloning and characterization of dullard: a novel gene required for neural development. Biochem Biophys Res Commun 295, 85 (2002).
11 Nieuwkoop, P. D. & Faber, J. Normal Table of Xenopus laevis (Daudin). (North Holland Pub. Co. 1956).
12 Sokol, S. Y. Analysis of Dishevelled signalling pathways during Xenopus development. Curr Biol 6, 1456 (1996).
13 Turner, D. L. & Weintraub, H. Expression of achaete-scute homolog 3 in Xenopus embryos converts ectodermal cells to a neural fate. Genes Dev 8, 1434 (1994).
14 Rupp, R. A., Snider, L. & Weintraub, H. Xenopus embryos regulate the nuclear localization of XMyoD. Genes Dev 8, 1311 (1994).

### Abbreviations:

5′UTR, 5′-untranslated region; AC, animal cap; APC, adenomatous polyposis coli; Atelo, atelocollagen; β-Cat, β-catenin; CIVIL, chronic myeloid leukemia; CNH, citron homology; Cont, control; GSK3β, glycogen synthase kinase 3β; GST, glutathione S-transferase; HA, hemagglutinin; IP, Immunoprecipitation; LEF, lymphoid enhancer factor; MO(s), morpholino oligonucleotide(s); N.D., not detected; n-βgal, nuclear β-galactosidase; NLS, nuclear localization signal; N.S., not significant; ORF, open reading frame; pSer, phosphoserine; RT, reverse transcription; shRNA, short hairpin RNA; siRNA, small interfering RNA; TCF(4), T-cell factor(−4); TNIK, Traf2- and Nck-interacting kinase; WT, wild type.

SUPPLEMENTARY TABLE S1
List of antibodies used in this study

| | | | | |
|---|---|---|---|---|
| Procedure | Antigen | Host | Cat. # | Supplier |

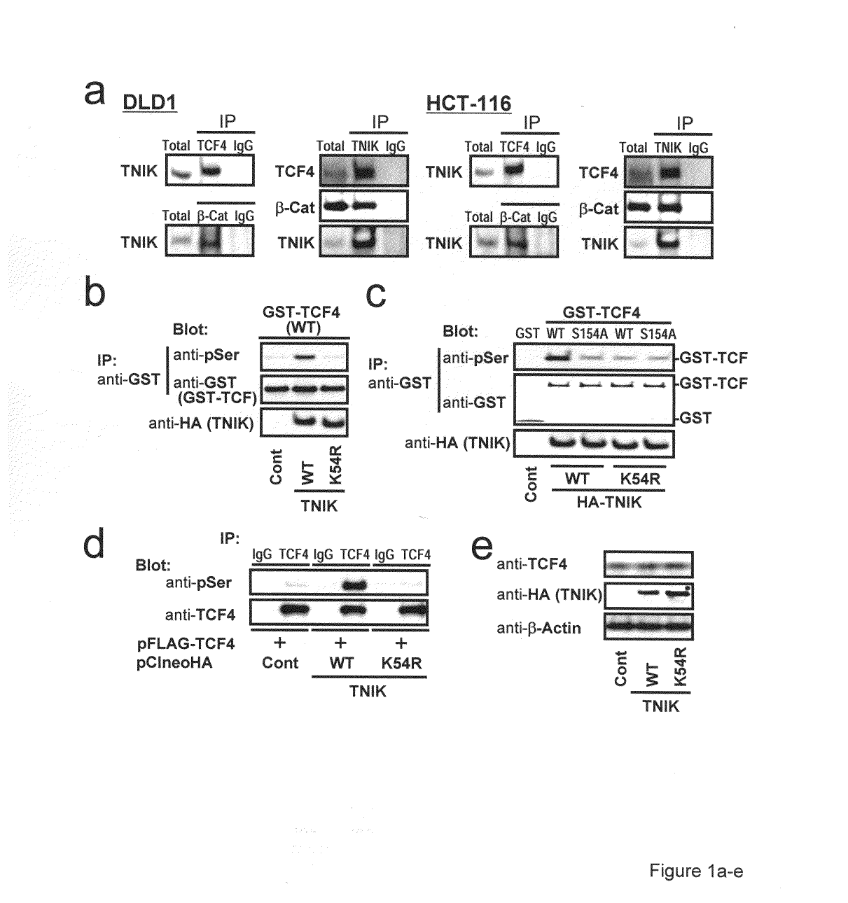

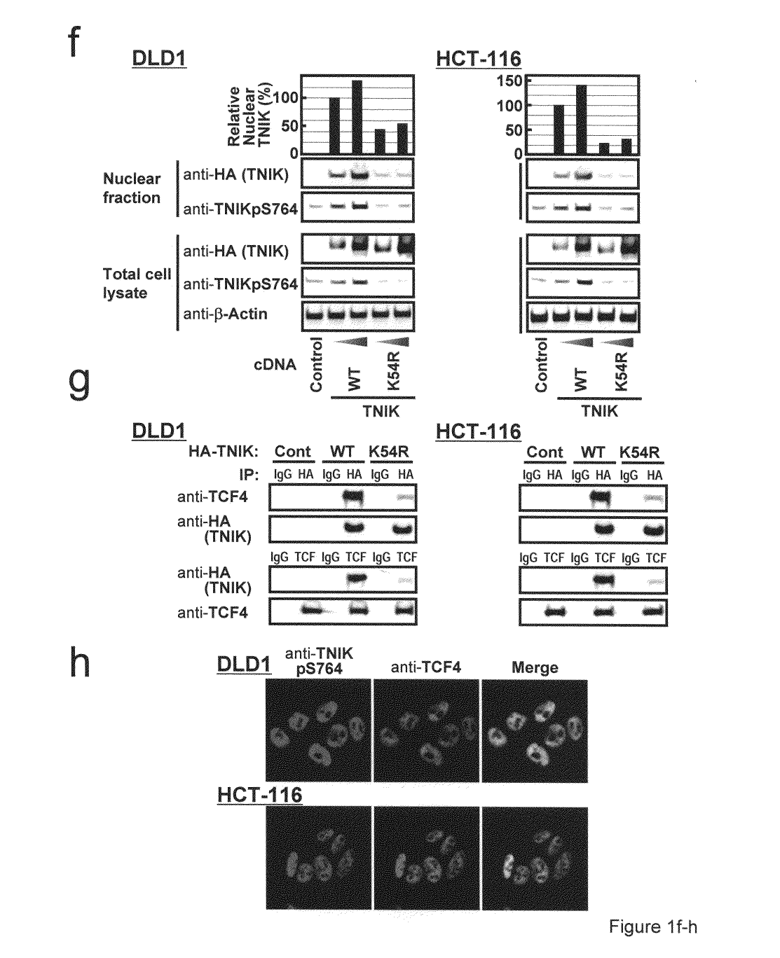

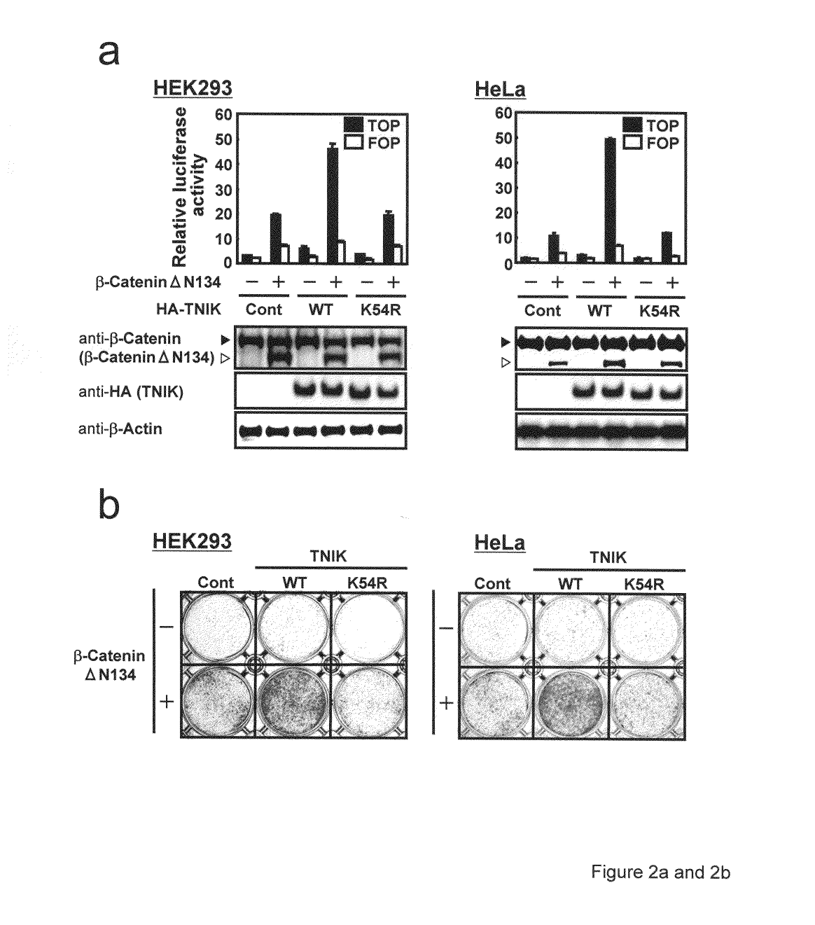

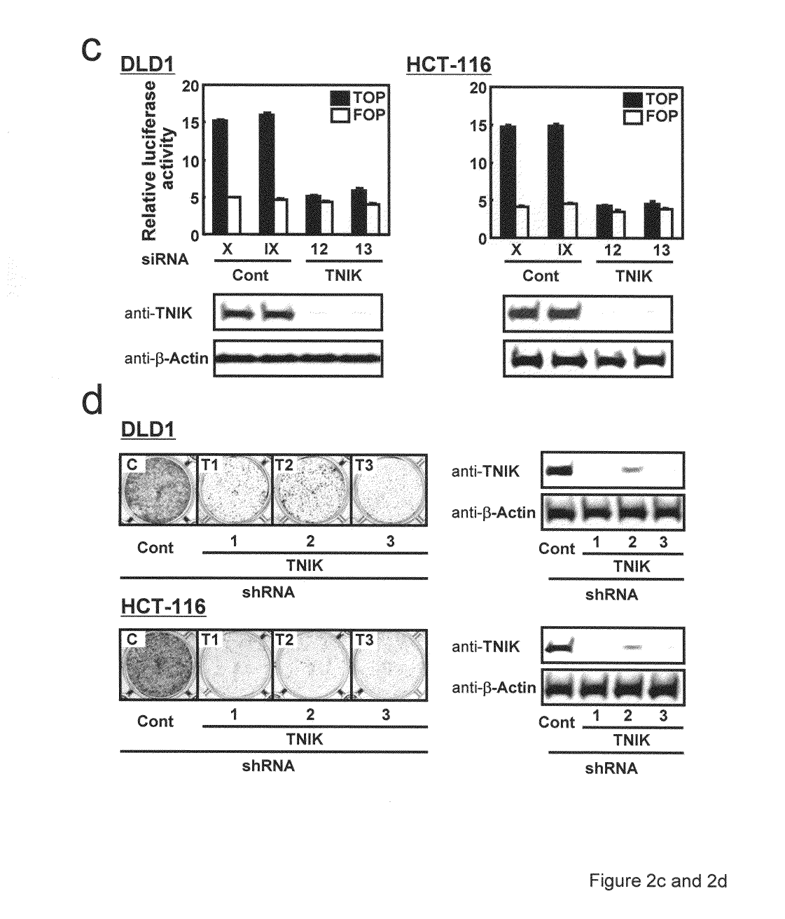

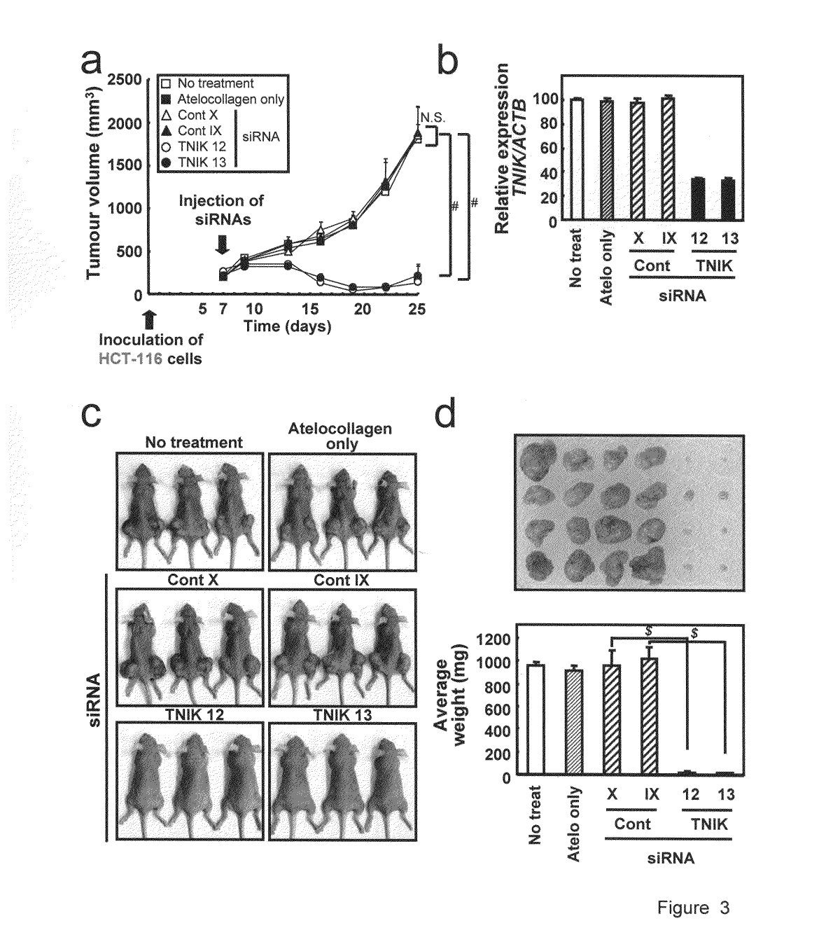

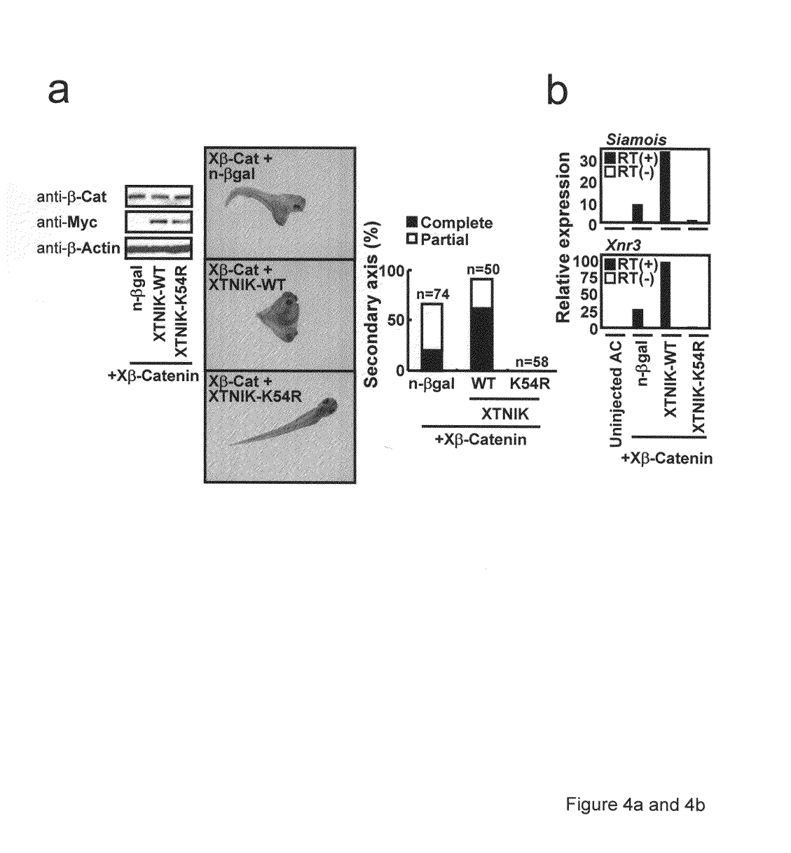

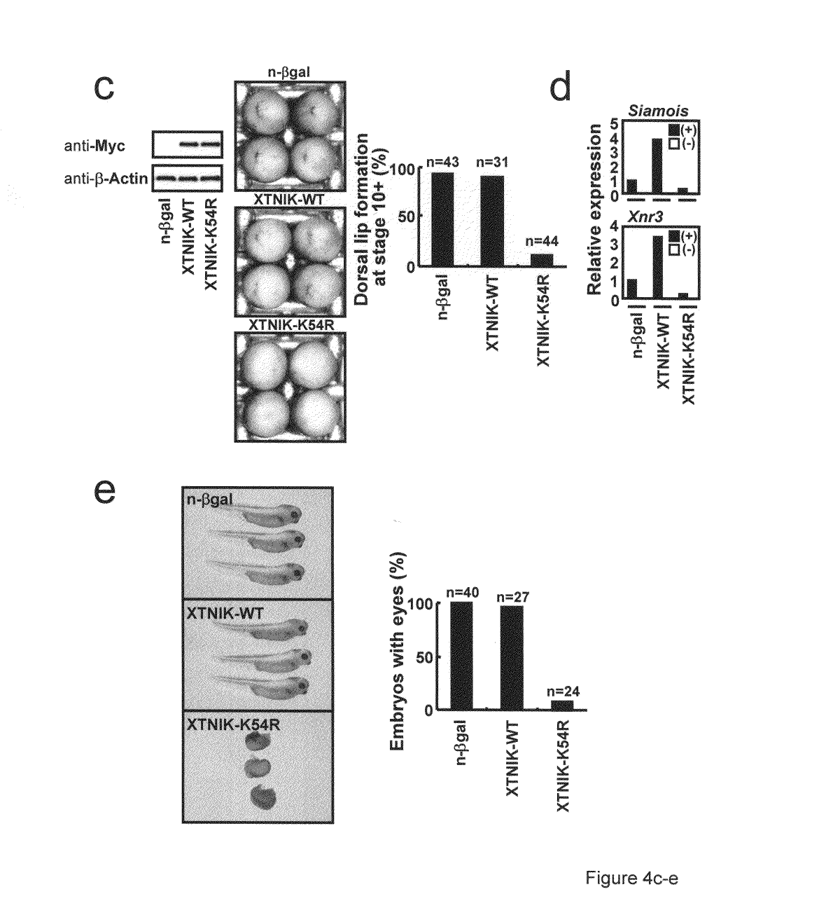

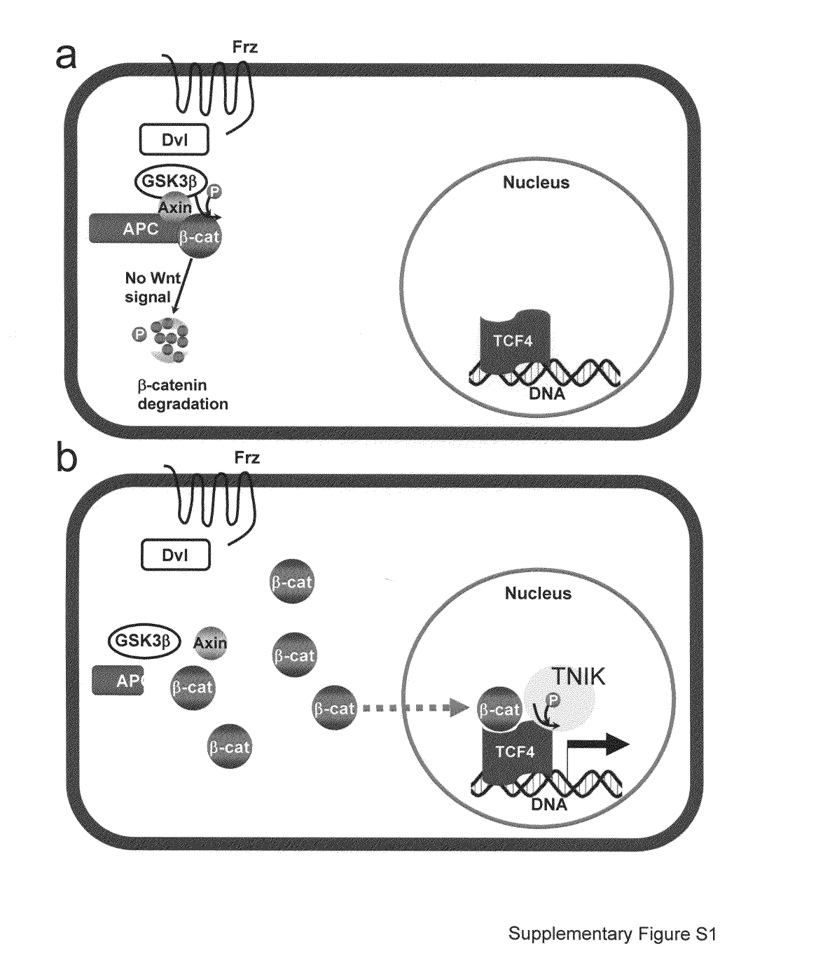

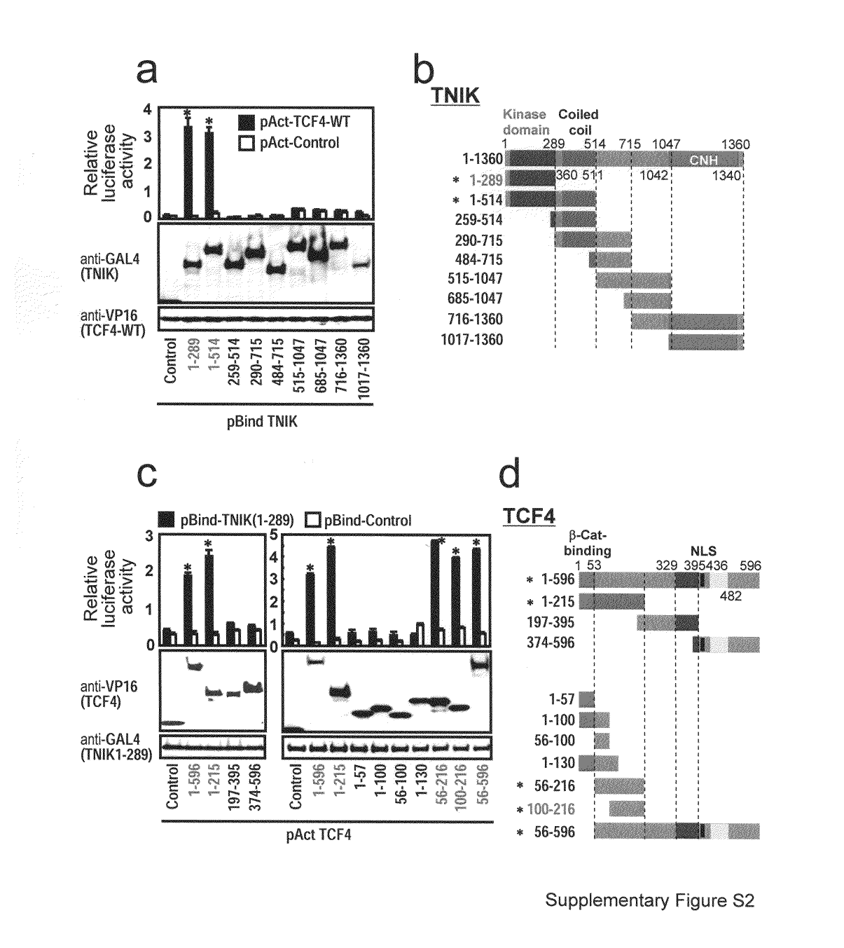

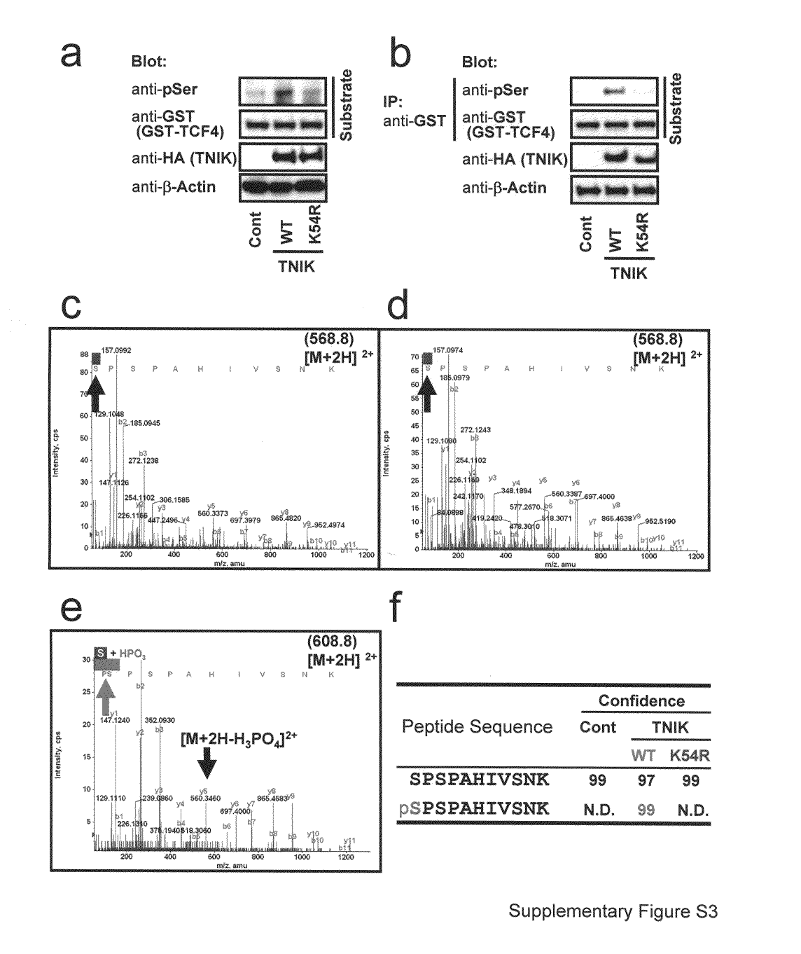

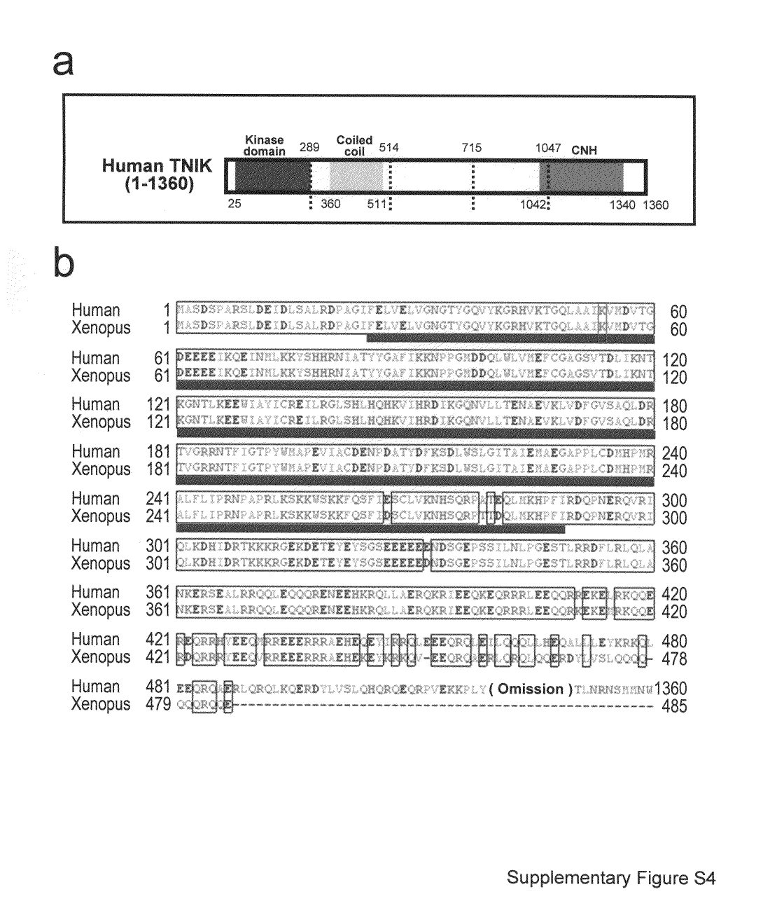

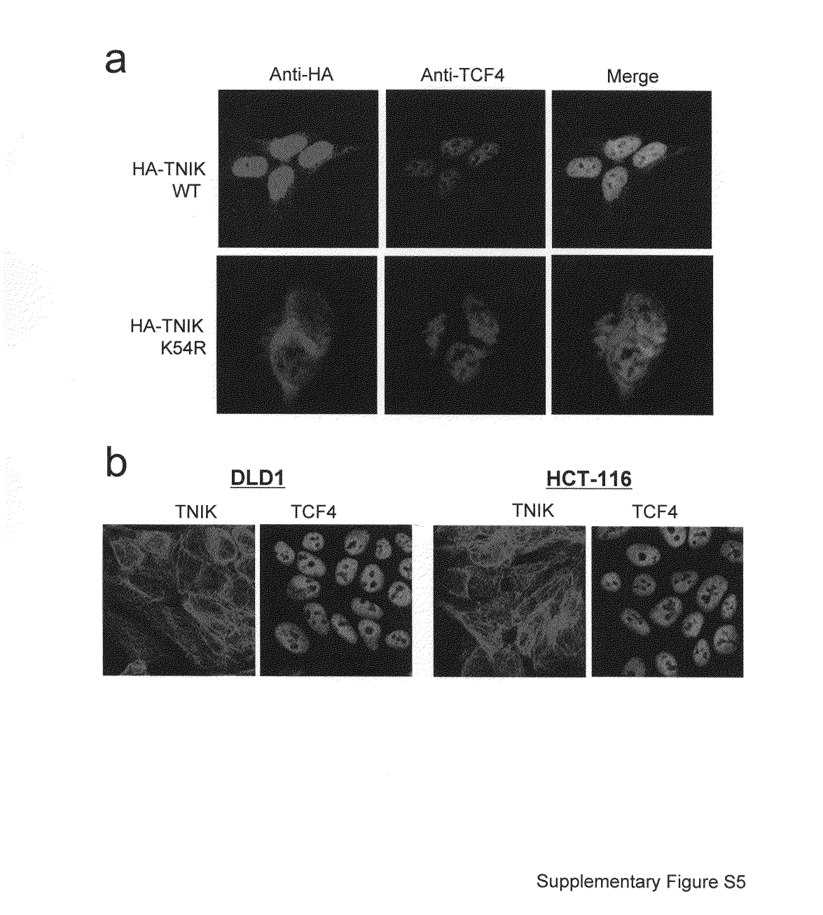

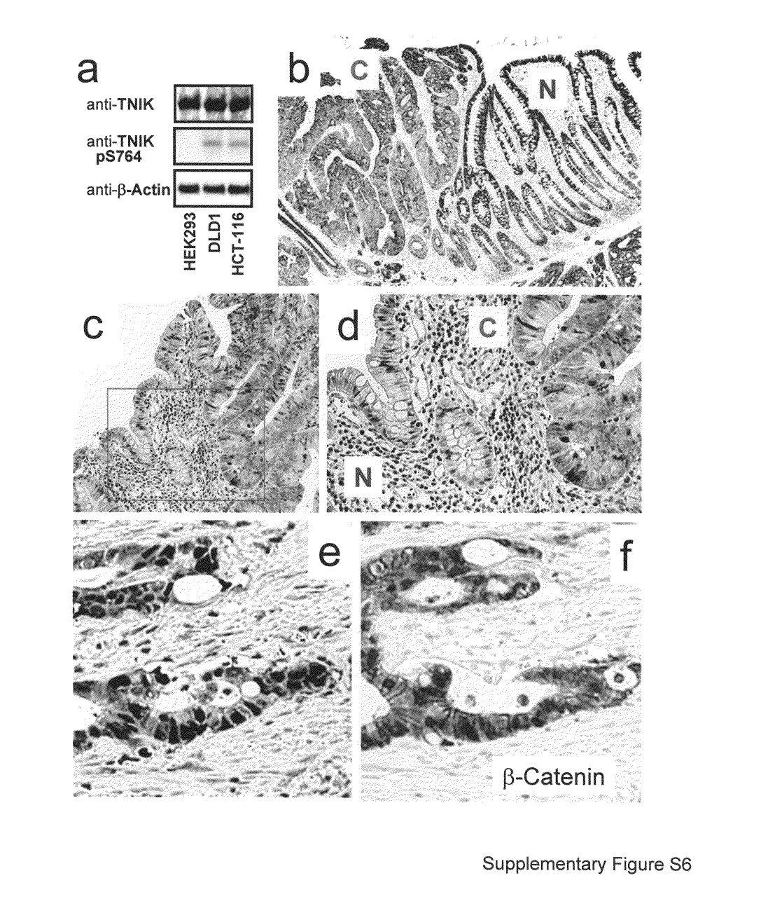

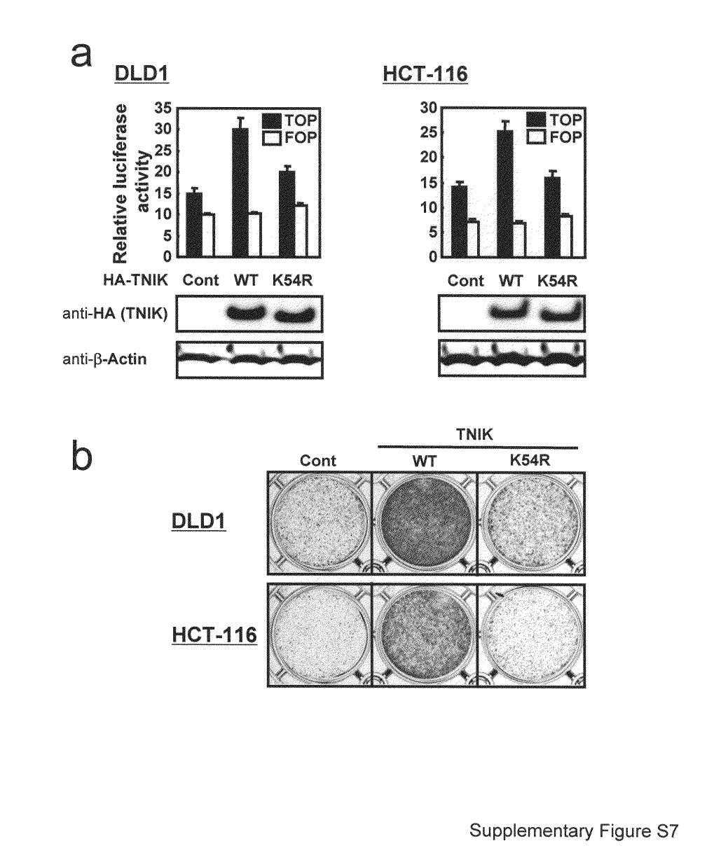

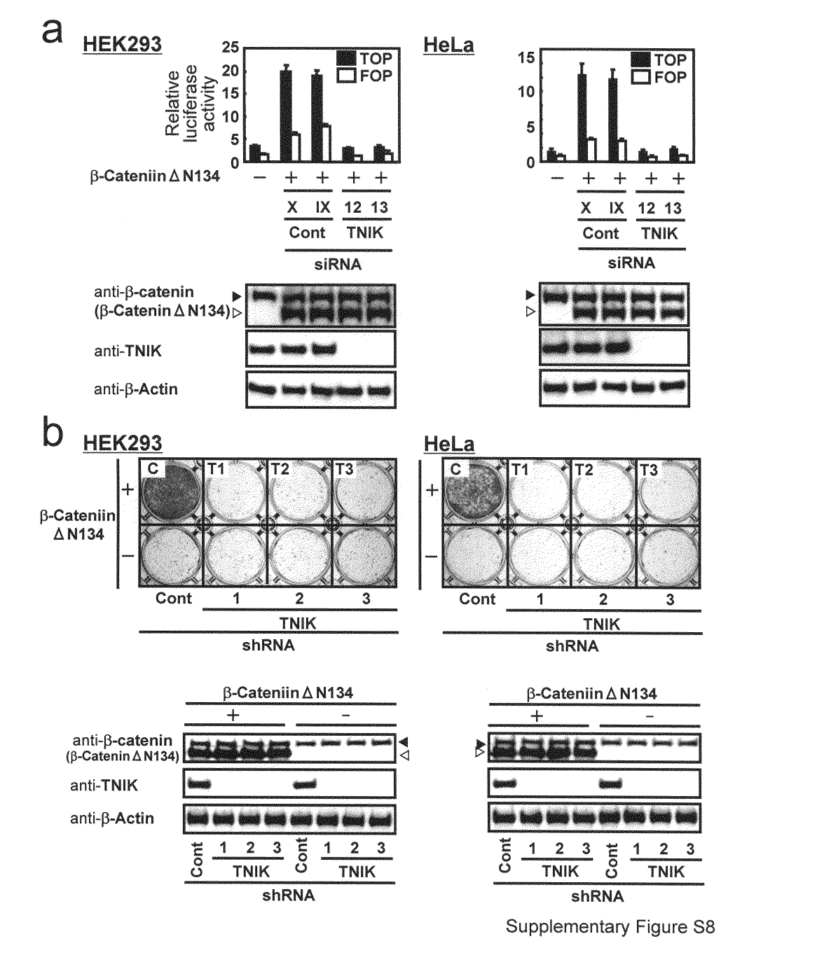

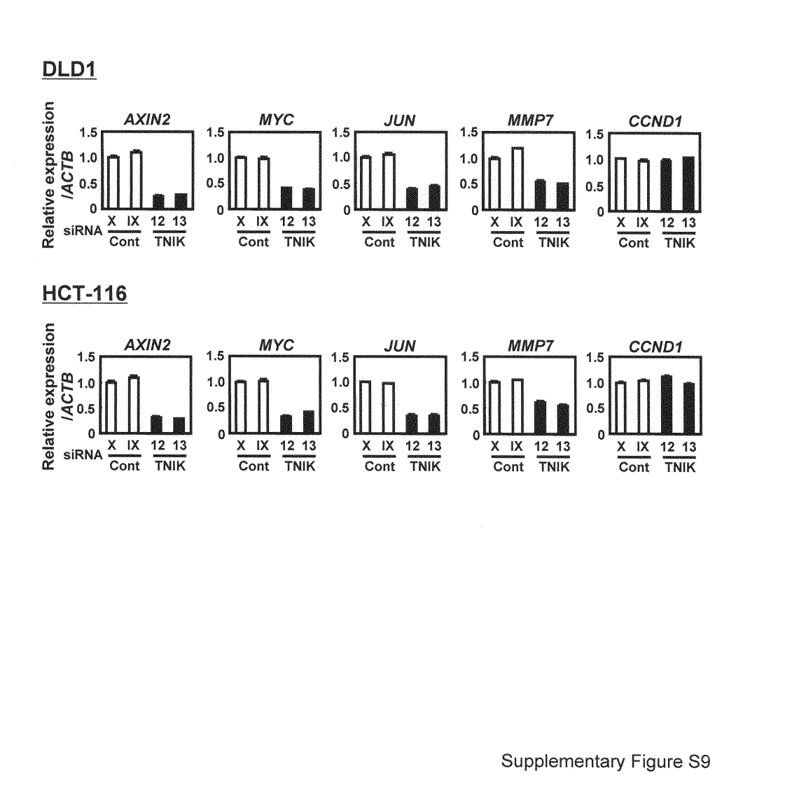

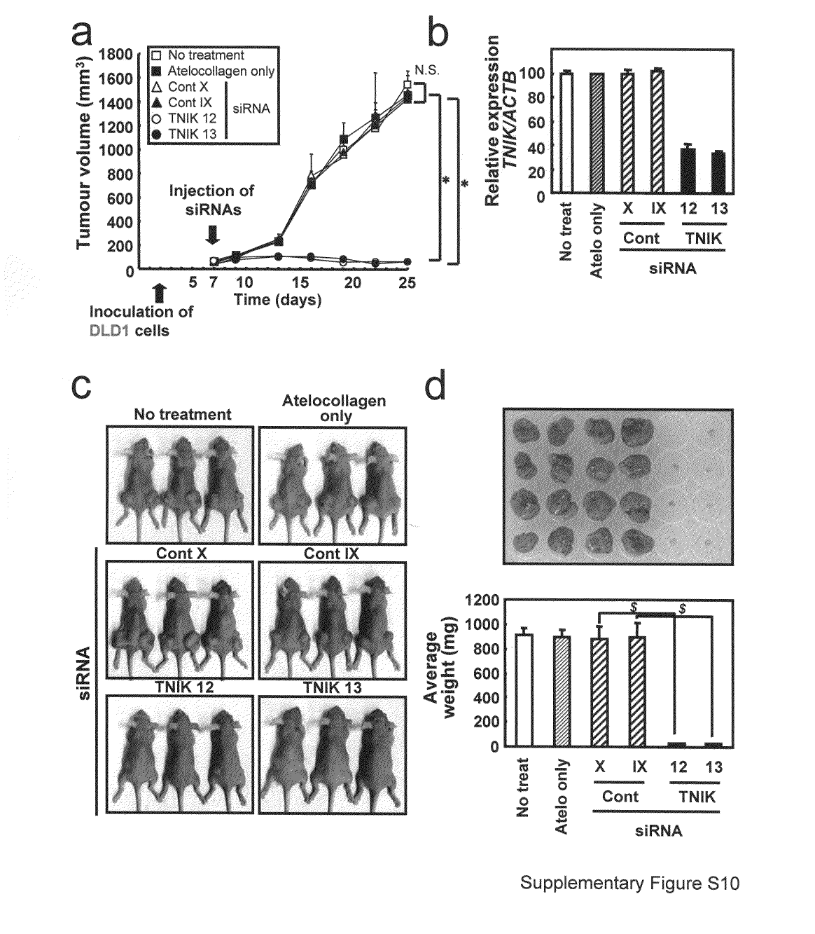

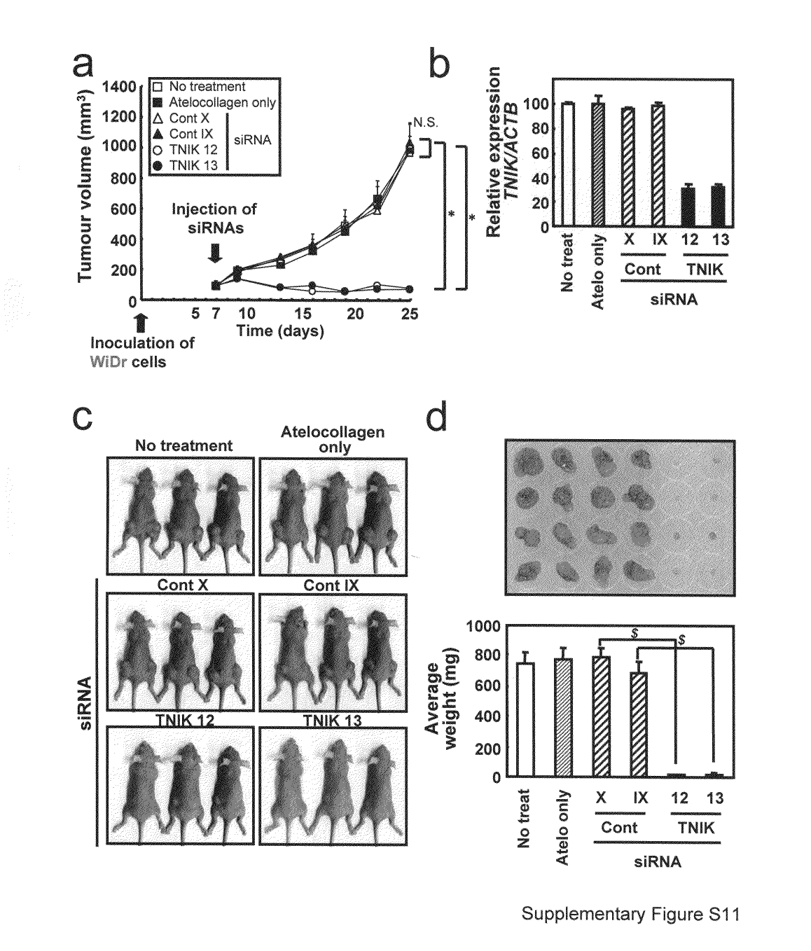

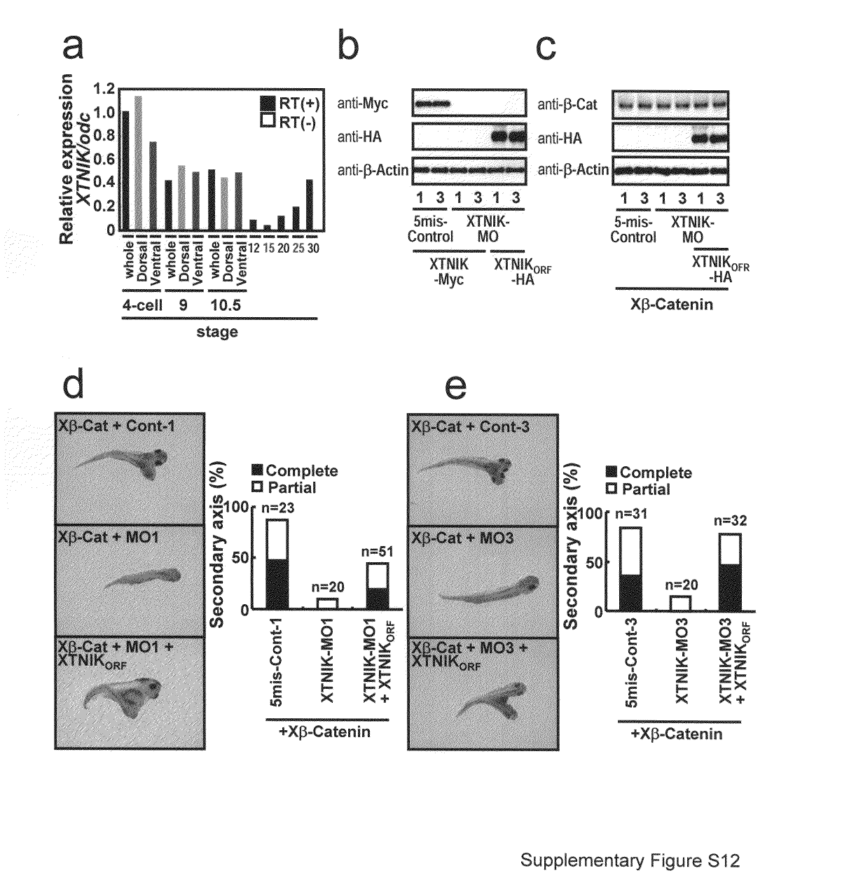

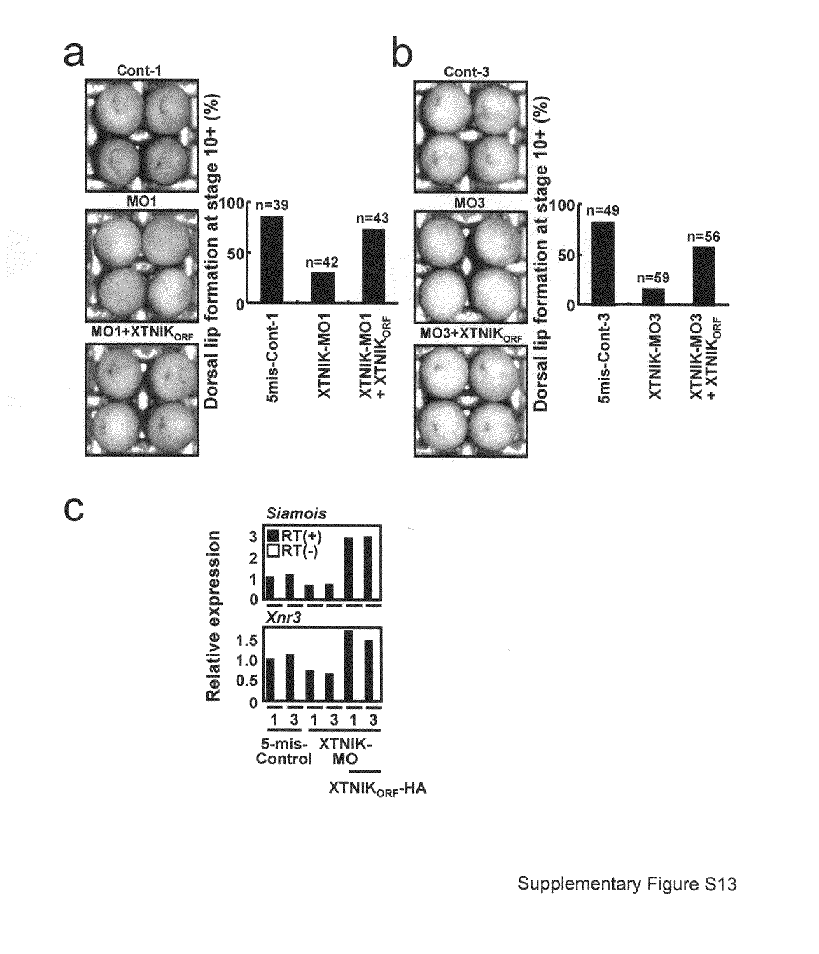

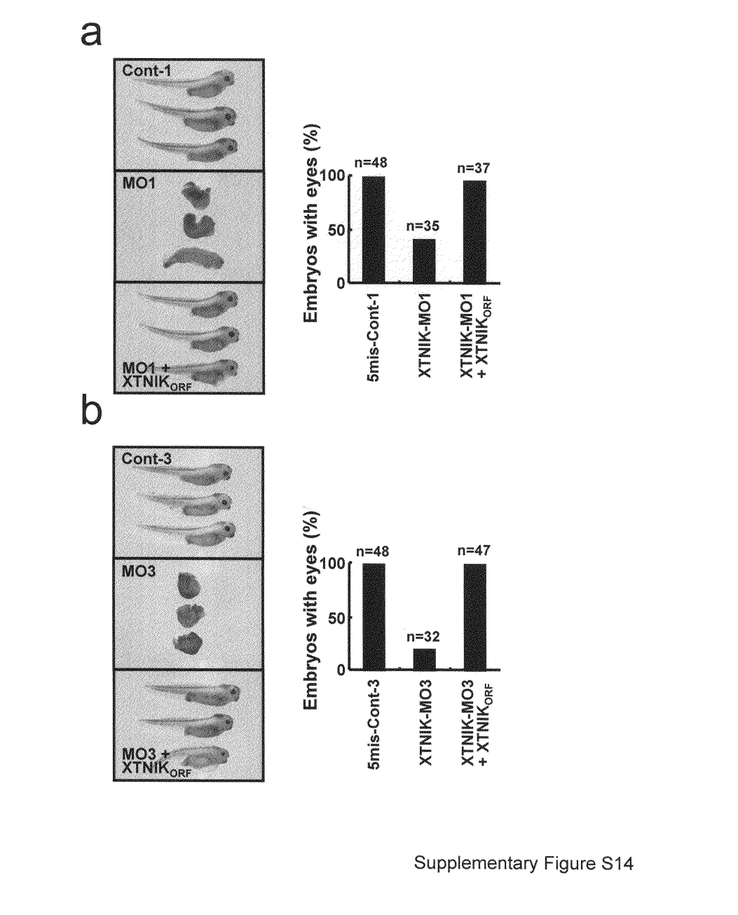

Antibodies used in the experiments shown in FIG. 1

| | | | | | |
|---|---|---|---|---|---|
| a | IP | TCF-4 | mouse | 6H5-3 | Upstate (Charlottesville, VA) |
| | | β-Catenin | mouse | clone14 | BD Transduction Laboratories (Palo Alto, CA) |
| | | TNIK | rabbit | GTX13141 | GeneTex (San Antonio, TX) |
| | Blot | TNIK | rabbit | GTX13141 | GeneTex (San Antonio, TX) |
| | | TCF-4 | mouse | 6H5-3 | Upstate (Charlottesville, VA) |
| | | β-Catenin | mouse | clone14 | BD Transduction Laboratories (Palo Alto, CA) |
| | | TNIK | mouse | 3D4 | Abnova (Taipei, Taiwan) |
| b-e | IP | GST | mouse | 13-6700 | Zymed (South San Francisco, CA) |
| | | TCF-4 | mouse | 6H5-3 | Upstate (Charlottesville, VA) |
| | Blot | Phospho-Serine | rabbit | ab9332 | Abcam (Cambridge, MA) |
| | | GST | rabbit | sc-459 | Santa Cruz Biotechnology (Santa Cruz, CA) |
| | | HA (hemagglutinin) | mouse | 12CA5 | Abgent (San Diego, CA) |
| | | TCF-4 | rabbit | sc-13027 | Santa Cruz Biotechnology (Santa Cruz, CA) |
| | | β-Actin | mouse | AC-74 | Sigma-Aldrich (St. Louis, MO) |
| f | Blot | HA (hemagglutinin) | mouse | 12CA5 | Abgent (San Diego, CA) |
| | | TNIKpS764 | rabbit | AP3276a | Abgent (San Diego, CA) |
| | | β-Actin | mouse | AC-74 | Sigma-Aldrich (St. Louis, MO) |
| g, h | IP | HA (hemagglutinin) | mouse | 12CA5 | Abgent (San Diego, CA) |
| | | TCF-4 | mouse | 6H5-3 | Upstate (Charlottesville, VA) |
| | Blot | TCF-4 | rabbit | sc-13027 | Santa Cruz Biotechnology (Santa Cruz, CA) |
| | | HA (hemagglutinin) | rabbit | sc-805 | Santa Cruz Biotechnology (Santa Cruz, CA) |
| | IF | TNIKpS764 | rabbit | AP3276a | Abgent (San Diego, CA) |
| | | TCF-4 | mouse | 6H5-3 | Upstate (Charlottesville, VA) |

Antibodies used in the experiments shown in FIG. 2

| | | | | |
|---|---|---|---|---|
| Blot | β-Catenin | mouse | clone14 | BD Transduction Laboratories (Palo Alto, CA) |
| | HA (hemagglutinin) | mouse | 12CA5 | Abgent (San Diego, CA) |
| | TNIK | rabbit | GTX13141 | GeneTex (San Antonio, TX) |
| | β-Actin | mouse | AC-74 | Sigma-Aldrich (St. Louis, MO) |

Antibodies used in the experiments shown in FIG. 4

| | | | | |
|---|---|---|---|---|
| Blot | β-Catenin | rabbit | sc-7199 | Santa Cruz Biotechnology (Santa Cruz, CA) |
| | Myc | mouse | 9E10 | Zymed (South San Francisco, CA) |
| | β-Actin | mouse | AC-15 | Abcam (Cambridge, MA) |

Antibodies used in the experiments shown in Supplementary FIG. S2

| | | | | |
|---|---|---|---|---|
| Blot | GAL4 | mouse | sc-510 | Santa Cruz Biotechnology (Santa Cruz, CA) |
| | VP16 | mouse | 2GV-4 | Euromedex (Souffelweyersheim, France) |

Antibodies used in the experiments shown in Supplementary FIG. S3

| | | | | |
|---|---|---|---|---|
| IP | GST | mouse | 13-6700 | Zymed (South San Francisco, CA) |
| Blot | Phospho-Serine | rabbit | ab9332 | Abcam (Cambridge, MA) |
| | GST | rabbit | sc-459 | Santa Cruz Biotechnology (Santa Cruz, CA) |
| | HA (hemagglutinin) | mouse | 12CA5 | Abgent (San Diego, CA) |
| | β-Actin | mouse | AC-74 | Sigma-Aldrich (St. Louis, MO) |

Antibodies used in the experiments shown in Supplementary FIG. S5

| | | | | |
|---|---|---|---|---|
| IF | HA (hemagglutinin) | rabbit | sc-805 | Santa Cruz Biotechnology (Santa Cruz, CA) |
| | TCF-4 | mouse | 6H5-3 | Upstate (Charlottesville, VA) |
| | TNIK | rabbit | GTX13141 | GeneTex (San Antonio, TX) |

Antibodies used in the experiments shown in Supplementary FIG. S6

| | | | | |
|---|---|---|---|---|
| Blot | TNIK | rabbit | GTX13141 | GeneTex (San Antonio, TX) |
| | TNIKpS764 | rabbit | AP3276a | Abgent (San Diego, CA) |
| | β-Actin | mouse | AC-74 | Sigma-Aldrich (St. Louis, MO) |
| IHC | TNIK | rabbit | GTX13141 | GeneTex (San Antonio, TX) |
| | TNIKpS764 | rabbit | AP3276a | Abgent (San Diego, CA) |
| | β-Catenin | mouse | clone14 | BD Transduction Laboratories (Palo Alto, CA) |

Antibodies used in the experiments shown in Supplementary FIG. S7

| | | | | |
|---|---|---|---|---|
| Blot | HA (hemagglutinin) | mouse | 12CA5 | Abgent (San Diego, CA) |
| | β-Actin | mouse | AC-74 | Sigma-Aldrich (St. Louis, MO) |

Antibodies used in the experiments shown in Supplementary FIG. S8

| | | | | |
|---|---|---|---|---|
| Blot | β-Catenin | mouse | clone14 | BD Transduction Laboratories (Palo Alto, CA) |
| | TNIK | rabbit | GTX13141 | GeneTex (San Antonio, TX) |
| | β-Actin | mouse | AC-74 | Sigma-Aldrich (St. Louis, MO) |

Antibodies used in the experiments shown in Supplementary FIG. S12

| | | | | |
|---|---|---|---|---|
| Blot | Myc | mouse | 9E10 | Zymed (South San Francisco, CA) |
| | HA (hemagglutinin) | mouse | 12CA5 | Abgent (San Diego, CA) |
| | β-Catenin | rabbit | sc-7199 | Santa Cruz Biotechnology (Santa Cruz, CA) |
| | β-Actin | mouse | AC-15 | Abcam (Cambridge, MA) |

IP, Immunoprecipitation; IF, Immunofluorescence; IHC, Immunohistochemistry.

### SUMMARY OF THE INVENTION

These inventors have found that, TNIK is the essential protein kinase in the Wnt signaling pathway, is deeply concerned with proliferation of cancer, especially a solid tumor, for example pancreatic cancer, non-small cell lung cancer, prostate cancer or breast cancer (especially colorectal cancer), and proliferation of cancer, especially a solid tumor (especially colorectal cancer) can be controlled by inhibiting the action of TNIK.

### DETAILED DESCRIPTION OF THE INVENTION

Based on the knowledge concerned, the present inventors have screened the compounds which have a TNIK inhibitory activity, have found that the aminothiazole derivative shown by the following general formula (I)

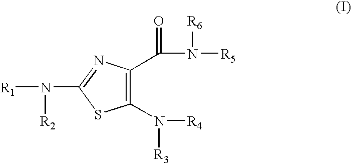

(wherein R1, R2, R3, R4, R5, and R6 independently represents a hydrogen atom or a substituent, respectively) or a pharmaceutically acceptable salt thereof has a TNIK inhibitory activity, have confirmed that the aminothiazole derivative suppresses proliferation of a cancer cell, and have completed this invention.

As a substituent in aminothiazole derivative (I), the following substituents can be mentioned, respectively.

As the substituent of R1 and R2, a substituted or unsubstituted alkyl group, a substituted or unsubstituted cycloalkyl group, a substituted or unsubstituted acyl group, a substituted or unsubstituted alkoxycarbonyl group, a substituted or unsubstituted carbamoyl group, a substituted or unsubstituted thiocarbamoyl group, a substituted or unsubstituted sulfonyl group, the substituted or unsubstituted heterocyclic ring, a substituted or unsubstituted aryl group, and substituted or unsubstituted heteroaromatic ring are mentioned.

As the substituent of R3 and R4, a substituted or unsubstituted alkyl group, a substituted or unsubstituted cycloalkyl group, a substituted or unsubstituted acyl group, a substituted or unsubstituted alkoxycarbonyl group, a substituted or unsubstituted carbamoyl group, a substituted or unsubstituted thiocarbamoyl group, a substituted or unsubstituted sulfonyl group, a substituted or unsubstituted heterocyclic ring, a substituted or unsubstituted aryl group, and a substituted or unsubstituted heteroaromatic ring are mentioned.

As the substituent of R5 and R6, a substituted or unsubstituted alkyl group, a substituted or unsubstituted cycloalkyl group, a substituted or unsubstituted heterocyclic ring, a substituted or unsubstituted aryl group, and a substituted or unsubstituted heteroaromatic ring are mentioned.

As the illustrative compound of aminothiazole derivative (I) or a pharmaceutically acceptable salt thereof, the following compounds or a pharmaceutically acceptable salt thereof are mentioned.
5-(4-acetamidobenzamido)-2-(phenylamino)thiazole-4-carboxamide
5-(3-methylbenzamido)-2-(phenylamino)thiazole-4-carboxamide
5-(2-fluorobenzamido)-2-(phenylamino)thiazole-4-carboxamide
5-(4-methoxybenzamido)-2-(phenylamino)thiazole-4-carboxamide
5-(3-methoxybenzamido)-2-(phenylamino)thiazole-4-carboxamide
2-(phenylamino)-5-(2-(thiophen-2-yl)acetamido)thiazole-4-carboxamide
5-(3-methylbutanamido)-2-(phenylamino)thiazole-4-carboxamide
5-(2-cyclopentylacetamido)-2-(phenylamino)thiazole-4-carboxamide
2-(p-toluidino)-5-(2-fluorobenzamido)thiazole-4-carboxamide
2-(p-toluidino)-5-(4-acetamidobenzamido)thiazole-4-carboxamide
2-(p-toluidino)-5-(2-chlorobenzamido)thiazole-4-carboxamide
2-(p-toluidino)-5-(3-bromobenzamido)thiazole-4-carboxamide
2-(p-toluidino)-5-(2,6-difluorobenzamido)thiazole-4-carboxamide
2-(p-toluidino)-5-(3,4-dimethoxybenzamido)thiazole-4-carboxamide
2-(p-toluidino)-5-(thiophene-2-carboxamido)thiazole-4-carboxamide
2-(p-toluidino)-5-(2-ethylbutanamido)thiazole-4-carboxamide
2-(ethylamino)-5-(8-methyl-2-phenylquinoline-4-carboxamido)thiazole-4-carboxamide

Each of thiazole derivative (I) of this invention or a pharmaceutically acceptable salt thereof are well-known compounds, and can also be received from TimTec (Delaware, USA), Aurora Fine Chemicals (California, USA), etc.

Moreover, thiazole derivative (I) or a pharmaceutically acceptable salt thereof can be manufactured also by the procedure of illustrating below.

In the manufacturing method shown below, if a desired substituent is changed under the conditions of the methods or is unsuitable for proceeding the method, it can be manufactured easily by adding the procedure usually used in synthetic organic chemistry, for example the well-known procedure such as protection or deprotection of a functional group (T. W. Greene, Protective Groups in Organic Synthesis 3rd Edition, John Wiley & Sons, Inc., 1999 references).

Moreover, if needed, an order of reaction processes such as substituent induction is also changeable arbitrarily.

Compound (1) can be obtained with, for example, the manufacturing method shown in a process 1.

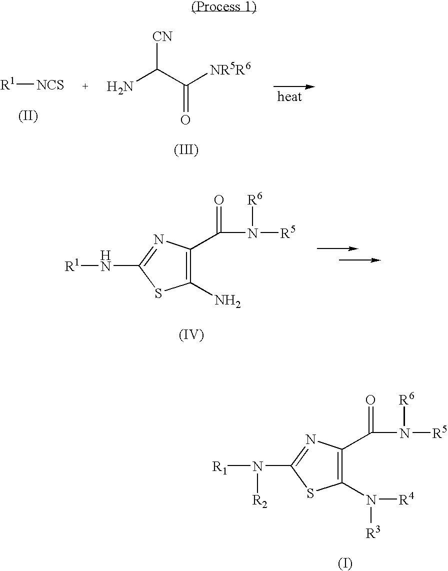

(R1, R2, R3, R4, R5, and R6 are as defined above.)

Compound (II) and (III) which are the starting materials of a process 1 be obtained as a commercial products (for example, Acros Organics product and URL:http://www.acros.com/), or obtained by either a well-known procedure or the procedure according to it.

Compound (IV) can be manufactured according to, for example, the procedure published in the paper (J. Chem. Soc. 1949, 3001 references) etc.

That is to say, compound (IV) can be obtained by carrying out the reaction of compound (II) and compound (III) among inert organic solvents, such as ethyl acetate.

Compound (I) can be obtained from compound (IV) by setting the conditions of acylation or alkylation well used in synthetic organic chemistry, if needed, repeating protection and deprotection of a functional group above mentioned.

Moreover, the pharmaceutically acceptable salt thereof illustrated below by the procedure well used in synthetic organic chemistry, if needed, can be obtained.

The pharmaceutically acceptable acid addition salt includes a salt with an inorganic acid such as hydrochloric acid, hydrobromic acid, sulfuric acid, or a salt with an organic acid such as maleic acid, fumaric acid, succinic acid, citric acid.

A further object of the present invention is to provide novel aminothiazole derivatives shown by the following general formula (I′).

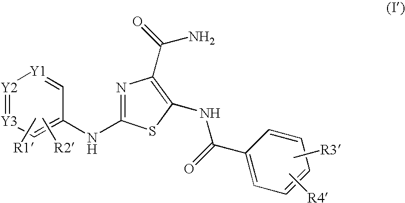

(wherein R1′ and R2′ independently represent a hydrogen atom, a halogen atom, a hydroxy group, a substituted or unsubstituted alkyl group, a substituted or unsubstituted alkoxy group, a substituted or unsubstituted amino group, an acylamino group, a nitro group, a substituted or unsubstituted alkoxycarbonylamino group, R3′ and R4′ independently represent a hydrogen atom, a halogen atom, a hydroxy group, a substituted or unsubstituted alkyl group, a substituted or unsubstituted alkoxy group, a substituted or unsubstituted amino group, a substituted or unsubstituted acylamino group or a substituted or unsubstituted alkyl sulfonamido group, and Y1, Y2 and Y3 independently represent a nitrogen atom or a carbon atom.), or a pharmaceutically acceptable salt thereof.

The substituent as used herein includes, for example, a halogen atom (such as F, Cl, Br), a substituted or unsubstituted C1-C8 alkyl group, a substituted or unsubstituted C1-C8 alkoxy group, a substituted or unsubstituted C1-C4 acylamino group, or C1-C2 alkyl sulfonamido group. The substituted or unsubstituted amino group as used herein includes, for example, dimethyl amino group, 4-methylpiperazin-1-yl group, 2-hydroxyethylamino group, 2-(dimethylamino)ethylamino group, 2-morpholinoethylamino group, 4-morpholino group, 2-(pyrrolidin-1-yl)ethylamino group, (2-hydroxyethyl)piperazin-1-yl group, 2-methoxyethylamino group, 2-aminoethylamino group, 4-(hydroxymethyl)piperidin-1-yl group, 2-(piperidin-1-yl)ethylamino group, 2-(pyridin-4-yl)ethylamino group or 2-(methylthio)ethylamino group. The substituted or unsubstituted C1-C8 alkoxy group as used herein includes, for example, methoxy group, benzyloxy group, 2-morpholinoethoxy group 2-(pyrrolidin-1-yl)ethoxy group or tetrahydro-2H-pyran-4-yloxy group. The substituted or unsubstituted acylamino group as used herein includes, for example, acetamido group, 2-hydroxyacetamido group, 2-(dimethylamino)acetamido group, 2-morpholinoacetamido group, 2-(pyrrolidin-1-yl)acetamido group, 2-(piperidin-1-yl)acetamido group or 2-(4-methylpiperazin-1-yl)acetamido group. The substituted or unsubstituted alkoxycarbonylamino group as used herein includes, for example, a substituted or unsubstituted C1-C8 alkoxycarbonylamino group (such as tert-butoxycarbonylamino group).

The following general reaction schemes detail the synthetic approaches to the aminothiazole derivatives disclosed herein. Compounds (I′) disclosed herein can be prepared as shown in Schemes 1-6 and as illustrated in the Examples by using standard synthetic methods and the starting materials, which are either commercially available or can be synthesized from commercially available precursors using synthetic methods known in the art, or variations thereof as appreciated by those skilled in the art.

Although these schemes often indicate exact structures, those skilled in the art will appreciate that the methods apply widely to analogous compounds of Formula I′, by being given appropriate consideration to protection and deprotection or reactive functional groups by methods standard to the art of organic chemistry. For example, hydroxy groups, in order to prevent unwanted side reactions, generally need to be converted to ethers or esters during chemical reactions at other sites in the molecule. The hydroxyl protecting group is readily removed to provide the free hydroxy group. Amino groups and carboxylic acid groups are similarly derivatized to protect them against unwanted side reactions. Typical protecting groups and methods for attaching and cleaving them are described fully by T. W. Greene, Protective Groups in Organic Synthesis 3rd Edition, John Wiley and Sons, Inc., New York (1999).

Each variable in the following schemes refers to any group consistent with the description of the compounds provided herein. Tautomers and solvates (e.g., hydrates) of the compounds of formula I are also within the scope of the invention.

Any compound of any formula disclosed herein can be obtained using procedures provided in the reaction Schemes, as well as procedures provided in the Examples, by selecting suitable starting materials and following analogous procedures. Thus, any compound of any formula disclosed or exemplified herein, can be obtained by using the appropriate starting materials and appropriate reagents, with the desired substitutions, and following procedures analogous to those described herein.

Compounds of Formula (I′) are generally synthesized by the formation of the amide from 5-aminothiazole intermediate (II′) and a substituted benzoyl chloride (III′-a), as shown in Scheme 1:

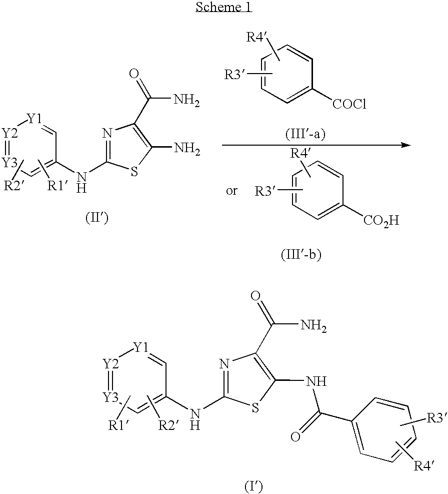

wherein R1′, R2′, R3′, R4′, Y1, Y2, and Y3 are the same as defined in the formula (I′).

The same type of amide-coupling reaction may be done with a substituted benzoic acid (III′-b) under general amide coupling conditions such as 1-ethyl-3-(3-dimethylaminopropyl) carbodiimide hydrochloride (EDC), hydroxybenzotriazole (HOBT) and a base such as diisopropylethylamine or triethylamine to afford the compounds of Formula I′.

In another approach, compounds of Formula (I′) may be prepared from the ester intermediate (IV′) by a direct aminolysis with ammonia, as shown in Scheme 2:

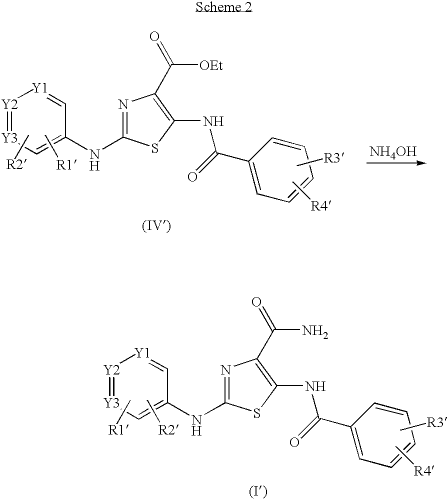

wherein R1′, R2′, R3′, R4′, Y1, Y2, and Y3 are the same as defined in the formula (I′).

The aminolysis reaction is carried out by using concentrated ammonium hydroxide solution or ammonia in methanol in presence of a solvent such as THF, or dioxane. The reaction is stirred and heated in a sealed tube at a temperature from 80° C. to 150° C., for 1-24 hours, preferably under microwave irradiation at 80° C. for 150 minutes using a microwave synthesizer.

The compounds represented by the formula (II′) in Scheme 1, which are used as starting materials of the amide-coupling reaction, may be prepared in a similar manner as described by Cook et al. (J. Chem. Soc. 1949, 3001). For example, the compounds represented by the formula (II′) may be prepared by the scheme 3 below:

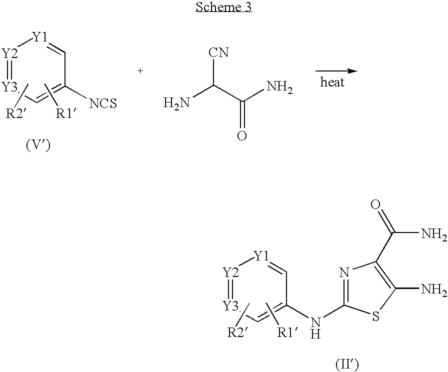

wherein R1′, R2′, Y1, Y2, and Y3 are the same as defined in the formula (I′).

Thus, a mixture of thioisocyanate (V′) and aminocyanoacetamide is stirred in a suitable solvent such as ethyl acetate, and heated to reflux condition for 0.5-2 hours to give the compounds represented by the formula (II′).

The thioisocyanate (V′) may be commercially available, or may be prepared from the corresponding amine by the methods well known in the field of organic synthesis, such as a thiophosgene treatment.

The substituted aminothiazole compounds (IV′) may be prepared via a palladium-catalyzed reaction with an aniline or amino-heteroaromatic compound (VII′) and 2-halogeno-thiazole compound (VI′), as shown in Scheme 4:

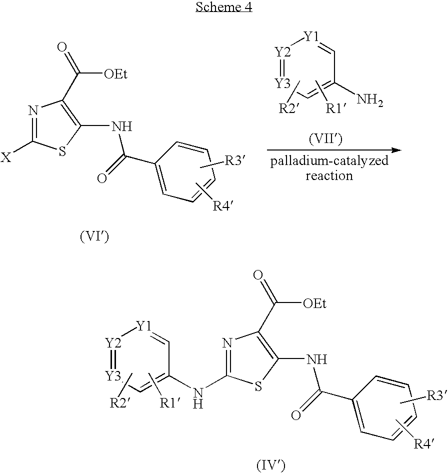

wherein R1′, R2′, R3′, R4′, Y1, Y2, and Y3 are the same as defined in the formula (I′) and X is a halogen selected from Cl, Br and I.

These Buckwald/Hartwig type reactions are well-known to those skilled in the art and are performed in inert solvents such as toluene, THF or dioxane and involve a palladium catalyst such as tris(dibenzylideneacetone)dipalladium (0), tetrakis(triphenylphosphine)palladium (0), palladium (II) acetate, and a base such as sodium, potassium or cesium carbonate and a ligand such as 4,5-Bis(diphenylphosphino)-9,9-dimethylxanthene (XANTPHOS). The same type of palladium-coupling reaction may be done with a corresponding halogeno-aromatic/heteroaromatic compound and a corresponding 2-aminothiazole analog to give the same desired aminothiazole intermediates (IV′).

The compound represented by the formula (VI′) may be prepared by the scheme 5 below:

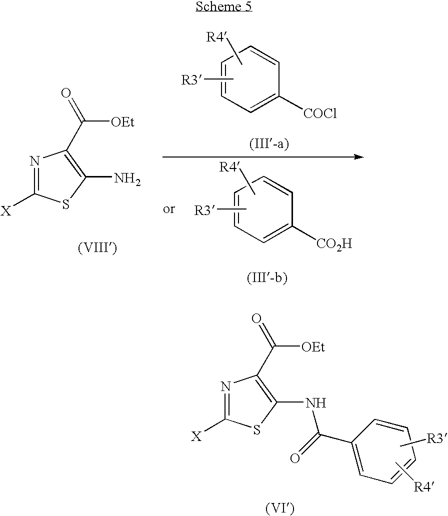

wherein R3′ and R4′ are the same as defined in the formula (I′) and X is a halogen selected from Cl, Br and I.

Thus, the compound represented by the formula (VI′) may be synthesized by the formation of the amide from 5-aminothiazole intermediate (VIII′) and a substituted benzoyl chloride (III′-a). The same type of amide-coupling reaction may be done with a substituted benzoic acid (III′-b) under general amide coupling conditions such as EDC, HOBT and a base such as diisopropylethylamine, or triethylamine.

The compound represented by the formula (VIII′) may be prepared from 5-aminothiazole-4-carboxylic acid ethyl ester by the scheme 6 below:

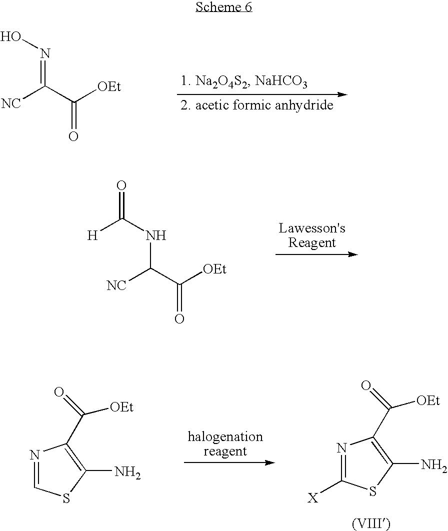

wherein X is a halogen selected from Cl, Br and I.

5-Aminothiazole-4-carboxylic acid ethyl ester is prepared according to the procedure described by Golankiewicz et al. (Tetrahedron, 41 (24), 5989-5994 (1985)). Thus, commercially available ethyl cyano(hydroxyimino)acetate is treated with sodium dithionate in sat. sodium bicarbonate aqueous solution to give ethyl 2-amino-2-cyanoacetate, which is then converted to the corresponding formamide with acetic formic anhydride. Subsequently, the obtained ethyl 2-cyano-2-formamidoacetate is treated with Lawesson's reagent, followed by treating with a halogenation reagent such as NCS, NBS to give the desired product.

The invention is further defined in the following Examples. It should be understood that these Examples are given by way of illustration only. From the above discussion and this Example, one skilled in the art can ascertain the essential characteristics of this invention, and without departing from the spirit and scope thereof, can make various changes and modifications to the invention to adapt the invention to various uses and conditions. As a result, the present invention is not limited by the illustrative examples set forth herein below, but rather defined by the claims appended hereto.

Specific examples of the compounds represented by the formula I′ are given in Table A-1 below:

TABLE A-1

| No. | | |
|---|---|---|
| (Ex. No.) | Structure | Name |
| A1 (Ex. 1) | 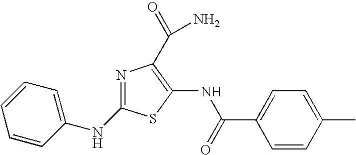 | 5-(4-methylbenzamido)-2- (phenylamino)thiazole- 4-carboxamide |
| A2 (Ex. 2) | 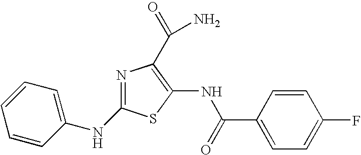 | 5-(4-fluorobenzamido)-2- (phenylamino)thiazole- 4-carboxamide |
| A3 (Ex. 3) | 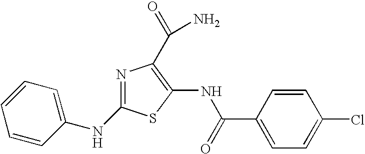 | 5-(4-chlorobenzamido)-2- (phenylamino)thiazole- 4-carboxamide |
| A4 (Ex. 4) | 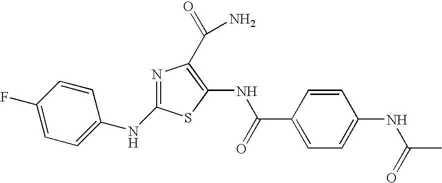 | 5-(4-acetami dobenzamido)-2- (4-fluorophenyl- amino)thiazole-4-carboxamide |
| A5 (Ex. 5) | 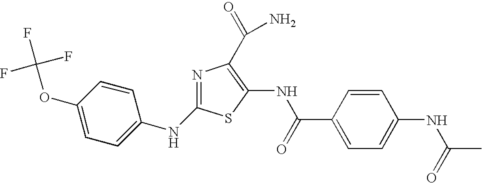 | 5-(4-acetamidobenzamido)-2-(4- (trifluoromethoxy) phenylamino) thiazole-4-carboxamide |
| A6 (Ex. 6) | 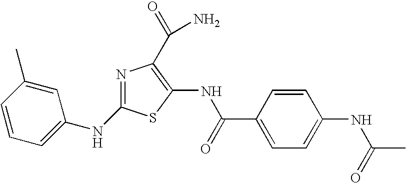 | 2-(m-toluidino)-5-(4- acetamidobenzamido) thiazole-4-carboxamide |
| A7 (Ex. 7) | 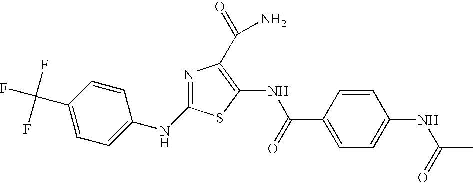 | 5-(4-acetamidobenzamido)-2- [4-(trifluoromethyl) phenylamino]thiazole-4- carboxamide |
| A8 (Ex. 8) | 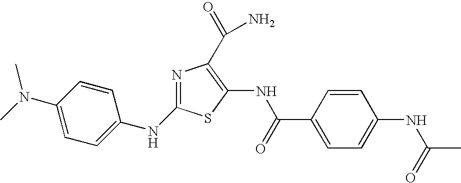 | 5-(4-acetamidobenzamido)-2- [4-(dimethylamino) phenylamino]thiazole-4- carboxamide |
| A9 (Ex. 9) | 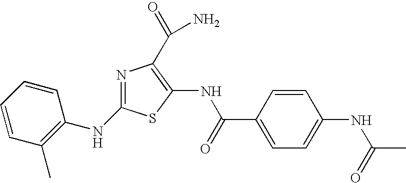 | 2-(o-toluidino)-5- (4-acetamidobenzamido) thiazole-4-carboxamide |
| A10 (Ex. 10) | 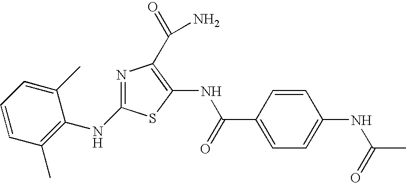 | 5-(4-acetamidobenzamido)-2- (2,6-dimethylphenyl- amino)thiazole-4-carboxamide |
| A11 (Ex. 11) | 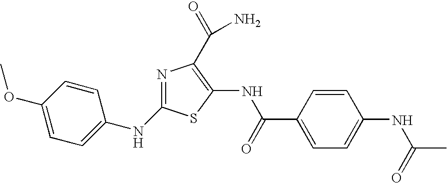 | 5-(4-acetamidobenzamido)-2- (4-methoxyphenyl amino)thiazole-4-carboxamide |
| A12 (Ex. 12) | 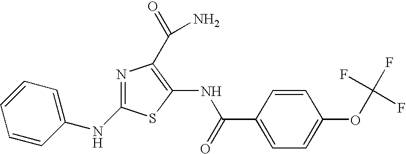 | 2-(phenylamino)-5-[4- (trifluoromethoxy) benzamido]thiazole-4- carboxamide |
| A13 (Ex. 13) | 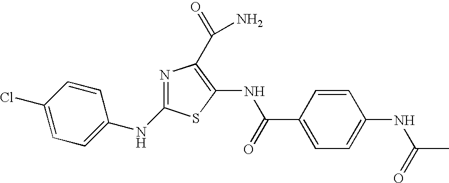 | 5-(4-acetamidobenzamido)-2- (4-chlorophenylamino) thiazole-4-carboxamide |
| A14 (Ex. 14) | 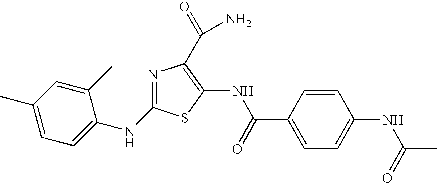 | 5-(4-acetamidobenzamido)-2- (4-chlorophenylamino) thiazole-4-carboxamide |
| A15 (Ex. 15) | 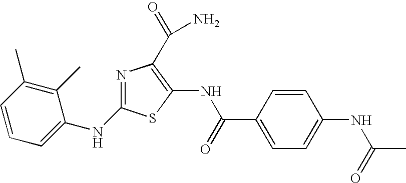 | 5-(4-acetamidobenzamido)-2- (2,4-dimethylphenyl- amino)thiazole-4-carboxamide |
| A16 (Ex. 16) | 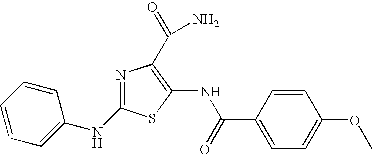 | 5-(4-methoxybenzamido)- 2-(phenylamino) thiazole-4-carboxamide |
| A17 (Ex. 17) | 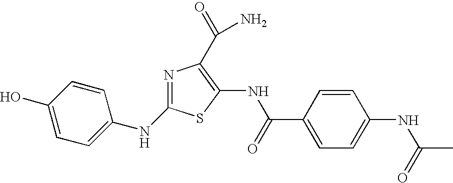 | 5-(4-acetamidobenzamido)-2- (4-hydroxyphenylamino) thiazole-4-carboxamide |
| A18 (Ex. 18) | 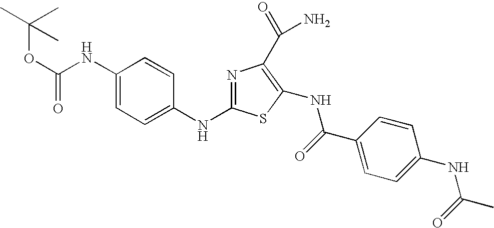 | tert-butyl 4-[5-(4-acetamidobenzamido)- 4-carbamoylthiazol- 2-ylamino]phenylcarbamate |
| A19 (Ex. 19) | 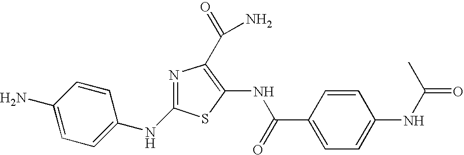 | 5-(4-acetamidobenzamido)- 2-(4-aminophenylamino) thiazole-4-carboxamide |
| A20 (Ex. 20) | 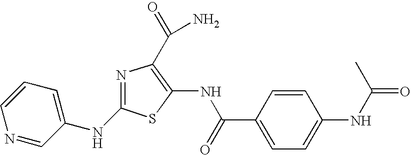 | 5-(4-acetamidobenzamido)- 2-(pyridin-3-ylamino) thiazole-4-carboxamide |
| A21 (Ex. 21) | 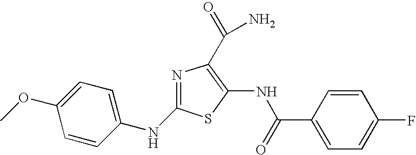 | 5-(4-fluorobenzamido)-2- (4-methoxyphenylamino) thiazole-4-carboxamide |
| A22 (Ex. 22) | 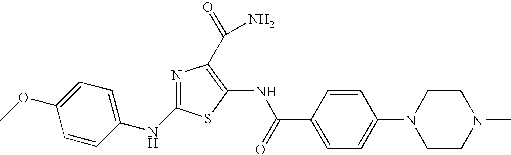 | 2-(4-methoxyphenylamino)- 5-[4-(4-methylpiperazin- 1-yl)benzamido]thiazole- 4-carboxamide |
| A23 (Ex. 23) | 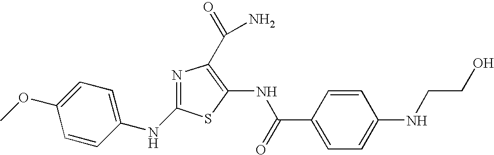 | 5-[4-(2-hydroxyethylamino) benzamido]-2-(4- methoxyphenylamino) thiazole-4-carboxamide |
| A24 (Ex. 24) | 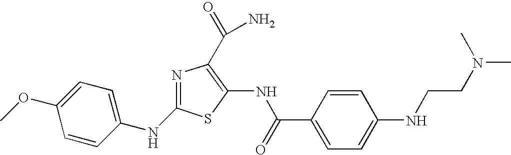 | 5-{4-[2-(dimethylamino) ethylamino]benzamido}- 2-(4-methoxyphenyl- amino)thiazole-4- carboxamide |
| A25 (Ex. 25) | 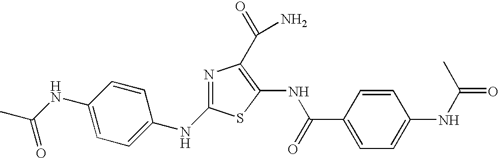 | 5-(4-acetamidobenzamido)- 2-(4-acetamidophenyl amino)thiazole-4-carboxamide |
| A26 (Ex. 26) | 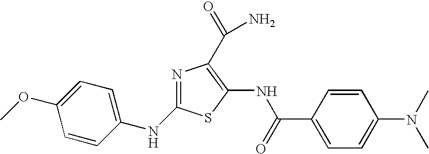 | 5-[4-(dimethylamino) benzamido]-2-(4-methoxy- phenylamino) thiazole-4-carboxamide |
| A27 (Ex. 27) | 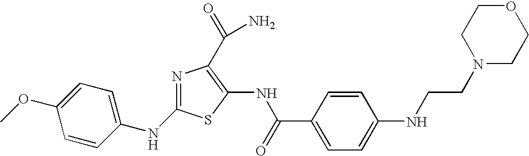 | 2-(4-methoxyphenylamino)- 5-[4-(2-morpholino- ethylamino)benzamido] thiazole-4-carboxamide |
| A28 (Ex. 28) | 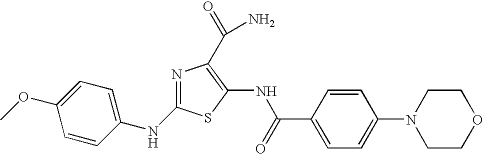 | 2-(4-methoxyphenylamino)- 5-(4-morpholinobenz- amido)thiazole-4-carboxamide |
| A29 (Ex. 29) | 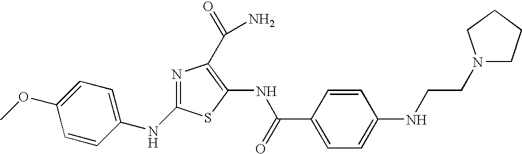 | 2-(4-methoxyphenylamino)- 5-{4-[2-(pyrrolidin-1- yl)ethylamino]benzamido} thiazole-4-carboxamide |
| A30 (Ex. 30) | 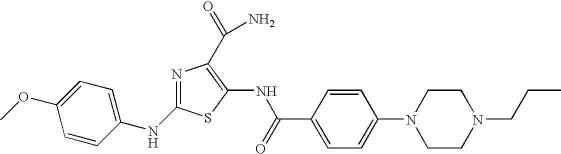 | 5-{4-[4-(2-hydroxyethyl) piperazin-1-yl]benzamido}- 2-(4-methoxyphenylamino) thiazole-4-carboxamide |
| A31 (Ex. 31) | 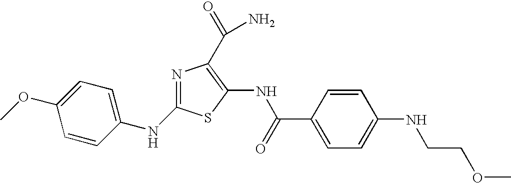 | 5-[4-(2-methoxyethylamino) benzamido]-2-(4- methoxyphenylamino) thiazole-4-carboxamide |
| A32 (Ex. 32) | 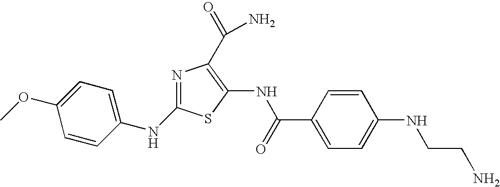 | 5-[4-(2-aminoethylamino) benzamido]-2- (4-methoxyphenylamino) thiazole-4-carboxamide |
| A33 (Ex. 33) | 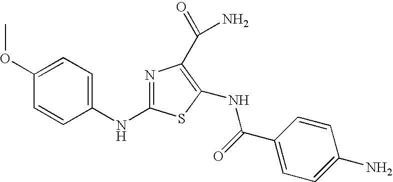 | 5-(4-aminobenzamido)-2- (4-methoxyphenylamino) thiazole-4-carboxamide |
| A34 (Ex. 34) | 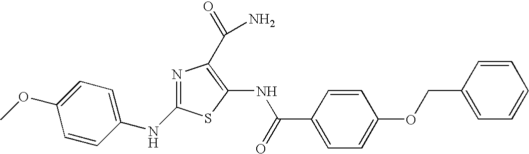 | 5-4-(benzyloxy)benzamido]- 2-(4-methoxyphenyl- amino)thiazole-4-carboxamide |
| A35 (Ex. 35) | 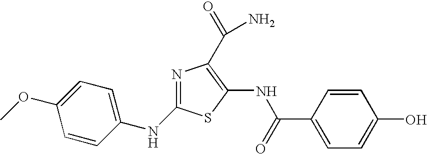 | 5-(4-hydroxybenzamido)- 2-(4-methoxyphenyl- amino)thiazole-4-carboxamide |
| A36 (Ex. 36) | 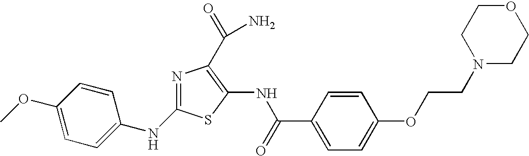 | 2-(4-methoxyphenylamino)- 5-[4-(2-morpholinoethoxy) benzamido]thiazole-4- carboxamide |
| A37 (Ex. 37) | 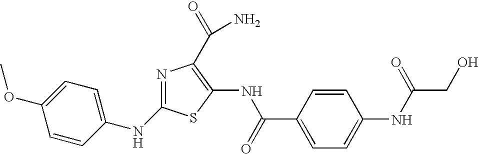 | [4-(2-hydroxyacetamido) benzamido]-2-(4-meth- oxyphenylamino) thiazole-4-carboxamide |
| A38 (Ex. 38) | 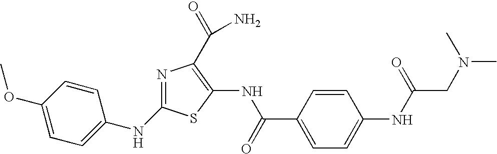 | 5-{4-[2-(dimethylamino) acetamido]benzamido}- 2-(4-methoxyphenylamino) thiazole-4-carboxamide |
| A39 (Ex. 39) | 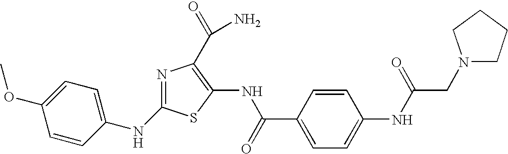 | 2-(4-methoxyphenylamino)- 5-[4-(2-(pyrrolidin-1- yl)acetamido)benzamido) thiazole-4-carboxamide |
| A40 (Ex. 40) | 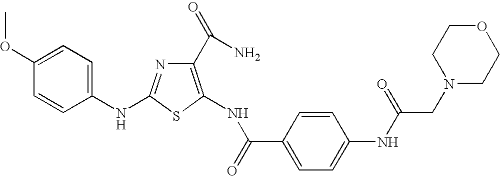 | 2-(4-methoxyphenylamino)-5- [4-(2-morpholinoacetamido) benzamido]thiazole-4- carboxamide |
| A41 (Ex. 41) | 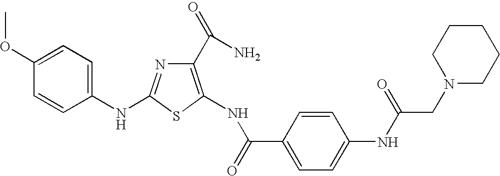 | 2-(4-methoxyphenylamino)- 5-{4-[2-(piperidin-1- yl)acetamido]benzamido} thiazole-4-carboxamide |
| A42 (Ex. 42) | 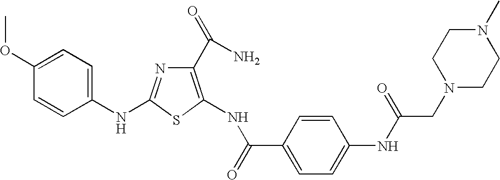 | 2-(4-methoxyphenylamino)-5- {4-[2-(4-methylpiperazin- 1-yl)acetamido]benzamido} thiazole-4-carboxamide |
| A43 (Ex. 43) | 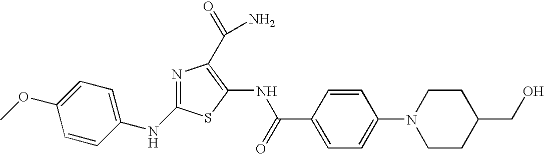 | 5-{4-[4-(hydroxymethyl) piperidin-1-yl]benzamido}- 2-(4-methoxyphenylamino) thiazole-4-carboxamide |
| A44 (Ex. 44) | 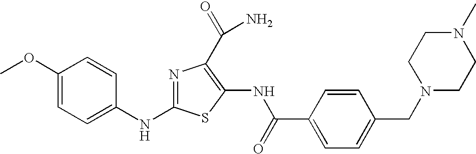 | 2-(4-methoxyphenylamino)-5- {4-[(4-methylpiperazin- 1-yl)methyl]benzamido} thiazole-4-carboxamide |
| A45 (Ex. 45) | 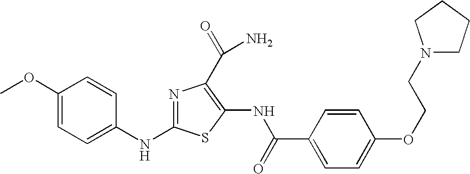 | 2-(4-methoxyphenylamino)- 5-{4-[2-(pyrrolidin-1- yl)ethoxy]benzamido} thiazole-4-carboxamide |
| A46 (Ex. 46) | 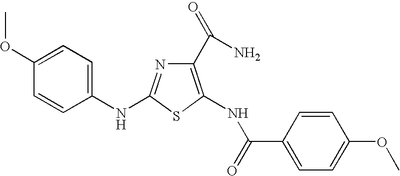 | 5-(4-methoxybenzamido)- 2-(4-methoxyphenylamino) thiazole-4-carboxamide |
| A47 (Ex. 47) | 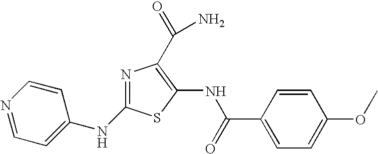 | 5-(4-methoxybenzamido)- 2-(pyridin-4-ylamino) thiazole-4-carboxamide |
| A48 (Ex. 48) | 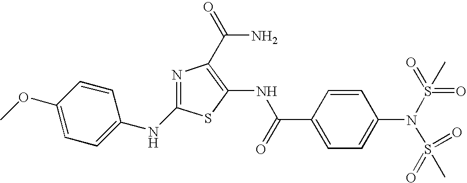 | 2-(4-methoxyphenylamino)- 5-{4-[N-(methylsulfonyl) methylsulfonamido] benzamido}thiazole- 4-carboxamide |
| A49 (Ex. 49) | 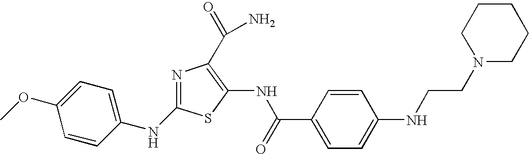 | 2-(4-methoxyphenylamino)- 5-{4-[2-(piperidin-1-yl) ethylamino]benzamido} thiazole-4-carboxamide |
| A50 (Ex. 50) | 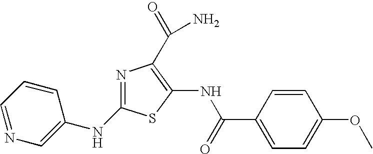 | 5-(4-methoxybenzamido)- 2-(pyridin-3-ylamino) thiazole-4-carboxamide |
| A51 (Ex. 51) | 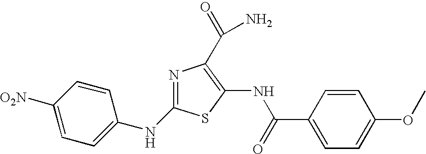 | 5-(4-methoxybenzamido)- 2-(4-nitrophenylamino) thiazole-4-carboxamide |
| A52 (Ex. 52) | 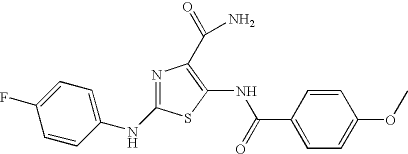 | 2-(4-fluorophenylamino)- 5-(4-methoxybenzamido) thiazole-4-carboxamide |
| A53 (Ex. 53) | 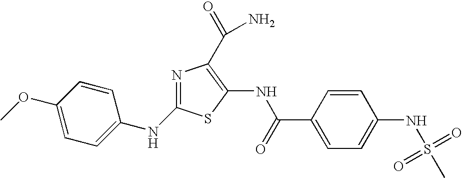 | 2-(4-methoxyphenylamino)- 5-[4-(methylsulfonamido) benzamido]thiazole- 4-carboxamide |
| A54 (Ex. 54) | 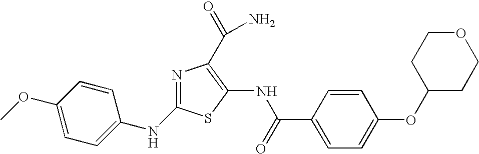 | 2-(4-methoxyphenylamino)- 5-[4-(tetrahydro-2H-pyran-4- yloxy)benzamido] thiazole-4-carboxamide |
| A55 (Ex. 55) | 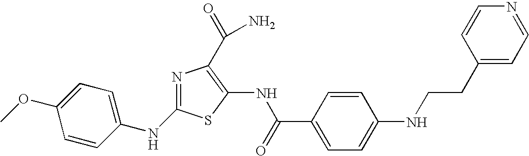 | 2-(4-methoxyphenylamino)- 5-{4-[2-(pyridin-4-yl) ethylamino]benzamido} thiazole-4-carboxamide |
| A56 (Ex. 56) | 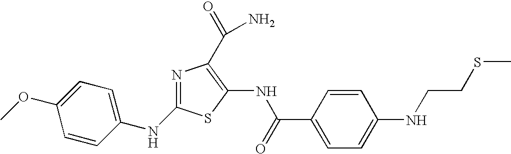 | 2-(4-methoxyphenylamino)- 5-{4-[2-(methylthio) ethylamino]benzamido} thiazole-4-carboxamide |
| A57 (Ex. 57) | 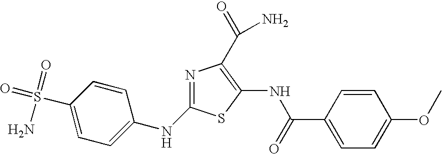 | 5-(4-methoxybenzamido)-2- (4-sulfamoylphenylamino) thiazole-4-carboxamide |

The aminothiazole derivatives (I) and aminothiazole derivatives (I′) show the TNIK inhibitory effects (Test Example 1) and do not show undesirable activity (Test Example 2).

The aminothiazole derivatives shows the anti-tumor activity (Test Example 3) and low toxicity.

The aminothiazole derivatives may be used as an anti-tumor agent in the form of a conventional pharmaceutical preparation for an oral or parenteral administration such as intravenous drip injection.

The preparation for oral administration includes solid preparations such as tablets, granules, powders, capsules, and liquid preparations such as syrups. These preparations can be prepared by a conventional method. The solid preparations can be prepared by using conventional pharmaceutical carriers, such as lactose, starch such as cornstarch, crystalline cellulose such as microcrystalline cellulose, hydroxypropyl cellulose, calcium carboxymethylcellulose, talc, magnesium stearate, etc. Capsules can be prepared by capsulating the granules or powders thus prepared. Syrups can be prepared by dissolving or suspending the aminothiazole derivatives in an aqueous solution containing sucrose, carboxymethylcellulose, etc.

The preparation for parenteral administration includes injections such as intravenous drip injection. The injection preparation can also be prepared by a conventional method, and optionally may be incorporateed in isotonic agents (e.g. mannitol, sodium chloride, glucose, sorbitol, glycerol, xylitol, fructose, maltose, mannose), stabilizers (e.g. sodium sulfite, albumin), preservatives (e.g. benzyl alcohol, methyl p-hydroxybenzoate).

The aminothiazole derivatives are effective for the treatment of tumors, especially solid tumors such as colorectal cancer, pancreatic cancer, non-small cell lung cancer, prostate cancer or breast cancer.

The dose of the aminothiazole derivatives may vary according to the severity of the diseases, ages and body weights of the patients, dosage forms and the like, but is usually in the range of 1 mg-1,000 mg per day in an adult, which may be administered once or by dividing into two or three times by the oral or parenteral route.

### TEST EXAMPLE

### Test Example 1

### Preparation of Recombinant Human TNIK (N-Terminal Segment)

A cDNA encoding the N-terminal segment (TNIK_N, residues 1-314) containing the kinase domain of human TNIK (NM_{—}015028.1) was amplified from cDNA mixture synthesized from human tissue (Biochain) by PCR using the following primers: 5′-AATTTCAGGGCGCCATGGCGAGCGACTCCCCGGCTCGAAG-3′ (forward primer, the underlined nucleotides indicates the location of a EheI site); 5′-ATTCGAAAGCGGCCGCTCATCCTCGCTTCTTCTTTGTTCTAT-3′ (reverse primer, the underlined nucleotides indicates the location of a NotI site). The cDNA was subcloned into baculovirus transfer vector pFastBac_GSTb that includes protease cleavage site and glutathione S-transferase purification tag (GST-tag). The plasmid was purified and the insertion of the pFastBac_GSTb-TNIK_N was confirmed by DNA sequencing. Then E. coli DH10Bac competent cells were transformed with the plasmid to prepare the recombinant bacmid according to the instructions for the Bac-to-Bac™ baculovirus expression systems (Invitrogen). The Sf9 cells were transfected with the recombinant bacmid containing pFastBac_GSTb-TNIK_N using Cellfectin Reagent (Invitrogen) in SF-900II serum free media (Invitrogen). The viral supernatant was collected from the medium 72 h after transfection. The virus was amplified three times by infecting actively growing Sf9 or Sf21 cells in Grace's insect media (Invitrogen) supplemented with 10% FCS and an antibiotic-antimycotic reagent (Invitrogen) for 72 h at 27° C. in T-flask or roller bottles. The titer of amplified TNIK_N virus was estimated at 2.36×10⁸ pfu/ml by using BacPAK™ Baculovirus Rapid Titer kit (Clontech).

Log-phase Sf21 cells (2×10⁶ cells/ml) in the Grace's insect media were infected with the recombinant baculovirus at MOI of 3.0 and incubated in roller bottles (250 ml media per bottle) for 72 h at 27° C., after which, the cells were collected by centrifugation, and the cell pellet washed with cold PBS and kept at −80° C. until purification. The following purification procedures were carried out at 4° C. The frozen cells were thawed on ice and lysed in lysis buffer (50 mM Tris-HCl, pH 7.5, 150 mM NaCl, 1% Nonidet P-40, 5 mM DTT, 0.5 mM EDTA, 0.5 mM EGTA) supplemented with 1 mM phenylmethansulfonylfluoride, 2 μg/ml leupeptin, 2 μg/ml aprotinin, 1 mM NaF, 100 μM sodium orthovanadate, and 1 μM cantharidin by sonication. The suspended lysate was cleared by centrifugation at 9000 g for 20 min and the supernatant was incubated for 1 h with glutathione Sepharose beads (GE Healthcare). The beads were suspended in buffer-H (50 mM Tris-HCl, pH 7.5, 1 M NaCl, 1 mM DTT, 0.5 mM EDTA, 0.5 mM EGTA and 0.05% Brij35) and washed with buffer-H followed by buffer-L (50 mM Tris-HCl, pH 7.5, 150 mM NaCl, 1 mM DTT, 0.5 mM EDTA, 0.5 mM EGTA, 0.05% Brij35) in an Econo-pack column (BIO-RAD). The bound TNIK_N was eluted with elution buffer (50 mM Tris-HCl, pH 8.0, 150 mM NaCl, 1 mM DTT, 10% glycerol, 0.5 mM EDTA, 0.5 mM EGTA and 5 mM reduced glutathione). The eluted fractions were collected and determined the protein concentration by Bradford reagent (BIO-RAD). The TNIK_N fractions were pooled and desalted using 10DG column (BIORAD) equilibrated with the storage buffer (50 mM Tris-HCl, pH 7.5, 150 mM NaCl, 1 mM DTT, 10% glycerol, 0.05% Brij35). The purified TNIK_N was characterized by electrophoresis using 4-20% polyacrylamide gels and matrix-assisted laser desorption/ionization reflection time-of-flight (MALDI-TOF) mass spectrometry on a Voyager-DE RP MALDI/TOF (Applied Biosystems). TNIK_N was confirmed by the molecular weight and MASCOT Peptide Mass Fingerprint.

### Kinase Assay:

The kinase assays were conducted in a 20 μl volume using 384-well plates (Greiner). The reaction mixture consists of compound or vehicle (1% DMSO), 0.08 ng/μl TNIK_N, 1 μM FITC-labeled substrate peptides, FITC-x-Lys-Tyr-Lys-Thr-Leu-Arg-Gln (x: ε-aminocaproic acid), 20 mM Hepes, pH 7.5, 0.01% Triton X-100, 5 mM MgCl₂, 25 μM ATP and 2 mM DTT. As blank, TNIK_N was excluded from the reaction mixture of vehicle (1% DMSO). The kinase reaction was carried out 1 h at room temperature and terminated by addition of 60 μl of the termination buffer (127 mM Hepes, pH 7.5, 26.7 mM EDTA, 0.01% Triton X-100, 1% DMSO and 0.13% Coating Reagent 3 (Caliper Life Sciences)). The amount of unphosphorylated and phosphorylated FITC-labeled substrate peptides was detected by Mobility Shift Micro Fluidic Technology (Caliper LC3000 System, Caliper Life Sciences). The kinase activity of TNIK_N was defined as P/(P+S) (P: peak height of the phosphorylated FITC-labeled substrate peptide; S: peak height of the FITC-labeled substrate peptide). Inhibition of the compounds was calculated as follows; inhibition (%)=(1−(A−C)/(B−C))×100 A: the mean P/(P+S) of compound wells; B: the mean P/(P+S) of vehicle wells; C: the mean P/(P+S) of blank wells. The IC50 values of the compound against the kinases were calculated from regression analysis of the log-concentration-inhibition curves.

### Result:

The test results are shown in Table 1.

TABLE 1

| NO. | Test Compound | IC50 (μM) |
|---|---|---|
| 1 | 5-(4-acetamidobenzamido)-2-(phenylamino)thiazole-4-carboxamide | 0.009 |
| 2 | 5-(3-methylbenzamido)-2-(phenylamino)thiazole-4-carboxamide | 0.040 |
| 3 | 5-(2-fluorobenzamido)-2-(phenylamino)thiazole-4-carboxamide | 0.019 |
| 4 | 5-(4-methoxybenzamido)-2-(phenylamino)thiazole-4-carboxamide | 0.010 |
| 5 | 5-(3-methoxybenzamido)-2-(phenylamino)thiazole-4-carboxamide | 7.7 |
| 6 | 2-(phenylamino)-5-(2-(thiophen-2-yl)acetamido)thiazole-4-carboxamide | 0.28 |
| 7 | 5-(3-methylbutanamido)-2-(phenylamino)thiazole-4-carboxamide | 0.18 |
| 8 | 5-(2-cyclopentylacetamido)-2-(phenylamino)thiazole-4-carboxamide | 0.13 |
| 9 | 2-(p-toluidino)-5-(2-fluorobenzamido)thiazole-4-carboxamide | 0.068 |
| 10 | 2-(p-toluidino)-5-(4-acetamidobenzamido)thiazole-4-carboxamide | 0.014 |
| 11 | 2-(p-toluidino)-5-(2-chlorobenzamido)thiazole-4-carboxamide | 1.7 |
| 12 | 2-(p-toluidino)-5-(3-bromobenzamido)thiazole-4-carboxamide | 1.7 |
| 13 | 2-(p-toluidino)-5-(2,6-difluorobenzamido)thiazole-4-carboxamide | 0.18 |
| 14 | 2-(p-toluidino)-5-(3,4-dimethoxybenzamido)thiazole-4-carboxamide | 0.057 |
| 15 | 2-(p-toluidino)-5-(thiophene-2-carboxamido)thiazole-4-carboxamide | 0.031 |
| 16 | 2-(p-toluidino)-5-(2-ethylbutanamido)thiazole-4-carboxamide | 4.1 |
| 17 | 2-(ethylamino)-5-(8-methyl-2-phenylquinoline-4-carboxamido)thiazole- | 3.1 |
| | 4-carboxamide | |
| A1 | 5-(4-methylbenzamido)-2-(phenylamino)thiazole-4-carboxamide | 0.006 |
| A2 | 5-(4-fluorobenzamido)-2-(phenylamino)thiazole-4-carboxamide | 0.017 |
| A4 | 5-(4-acetamidobenzamido)-2-(4-fluorophenylamino)thiazole-4-carboxamide | 0.018 |
| A9 | 2-(o-toluidino)-5-(4-acetamidobenzamido)thiazole-4-carboxamide | 0.018 |
| A11 | 5-(4-acetamidobenzamido)-2-(4-methoxyphenylamino)thiazole-4-carboxamide | 0.014 |
| A19 | 5-(4-acetamidobenzamido)-2-(4-aminophenylamino)thiazole-4-carboxamide | 0.024 |
| A20 | 5-(4-acetamidobenzamido)-2-(pyridin-3-ylamino)thiazole-4-carboxamide | 0.009 |
| A22 | 2-(4-methoxyphenylamino)-5-[4-(4-methylpiperazin-1- | 0.015 |
| | yl)benzamido]thiazole-4-carboxamide | |
| A23 | 5-[4-(2-hydroxyethylamino)benzamido]-2-(4- | 0.008 |
| | methoxyphenylamino)thiazole-4-carboxamide | |
| A24 | 5-{4-[2-(dimethylamino)ethylamino]benzamido}-2-(4- | 0.011 |
| | methoxyphenylamino)thiazole-4-carboxamide | |
| A27 | 2-(4-methoxyphenylamino)-5-[4-(2- | 0.009 |
| | morpholinoethylamino)benzamido]thiazole-4-carboxamide | |
| A29 | 2-(4-methoxyphenylamino)-5-{4-[2-(pyrrolidin-1- | 0.008 |
| | yl)ethylamino]benzamido}thiazole-4-carboxamide | |
| A31 | 5-[4-(2-methoxyethylamino)benzamido]-2-(4- | 0.010 |
| | methoxyphenylamino)thiazole-4-carboxamide | |
| A37 | 5-[4-(2-hydroxyacetamido)benzamido]-2-(4- | 0.009 |
| | methoxyphenylamino)thiazole-4-carboxamide | |
| A42 | 2-(4-methoxyphenylamino)-5-{4-[2-(4-methylpiperazin-1- | 0.018 |
| | yl)acetamido]benzamido}thiazole-4-carboxamide | |
| A43 | 5-{4-[4-(hydroxymethyl)piperidin-1-yl]benzamido}-2-(4- | 0.013 |
| | methoxyphenylamino)thiazole-4-carboxamide | |
| A44 | 2-(4-methoxyphenylamino)-5-{4-[(4-methylpiperazin-1- | 0.011 |
| | yl)methyl]benzamido}thiazole-4-carboxamide | |
| A45 | 2-(4-methoxyphenylamino)-5-{4-[2-(pyrrolidin-1- | 0.022 |
| | yl)ethoxy]benzamido}thiazole-4-carboxamide | |
| A46 | 5-(4-methoxybenzamido)-2-(4-methoxyphenylamino)thiazole-4-carboxamide | 0.016 |
| A47 | 5-(4-methoxybenzamido)-2-(pyridin-4-ylamino)thiazole-4-carboxamide | 0.003 |
| A48 | 2-(4-methoxyphenylamino)-5-{4-[N- | 0.006 |
| | (methylsulfonyl)methylsulfonamido]benzamido}thiazole-4- | |
| | carboxamide | |
| A49 | 2-(4-methoxyphenylamino)-5-{4-[2-(piperidin-1- | 0.005 |
| | yl)ethylamino]benzamido}thiazole-4-carboxamide | |
| A55 | 2-(4-methoxyphenylamino)-5-{4-[2-(pyridin-4- | 0.010 |
| | yl)ethylamino]benzamido}thiazole-4-carboxamide | |

### Test Example 2

### Selectivity Profiling Test

The inhibitory effects of Compound 1 against 20 tyrosine kinases and 30 serine/threonine kinases were investigated using QuickScout™ TK and STK Screening Panel (Carna Biosciences, Kobe Japan). The IC50 values of Compound 1 are showed in Table 2. The results reveal that Compound 1 inhibits TNIK_N more potently (IC50; 9 nM) than the other 50 kinases.

TABLE 2
Selectivity Profiling of Compound 1

| Tyrosine | | Serine/threonine | |
|---|---|---|---|
| kinases | IC50 (nM) | kinases | IC50 (nM) |
| KDR | 59 | MLK1 | 18 |
| EGFR | 69 | GSK3b | 120 |
| FLT3 | 95 | AurA | 130 |
| PDGFRa | 150 | CaMK4 | >300 |
| ABL | 290 | CDK2 | >300 |
| FGFR1 | >300 | CHK1 | >300 |
| EphA2 | >300 | CK1e | >300 |
| IGF1R | >300 | DAPK1 | >300 |
| ITK | >300 | DYRK1B | >300 |
| JAK3 | >300 | Erk2 | >300 |
| CSK | >300 | AKT1 | >300 |
| LCK | >300 | IKKb | >300 |
| MET | >300 | IRAK4 | >300 |
| EphB4 | >300 | MAPKAPK2 | >300 |
| PYK2 | >300 | MST1 | >300 |
| SRC | >300 | NEK2 | >300 |
| SYK | >300 | p38a | >300 |
| TIE2 | >300 | p70S6K | >300 |
| TRKA | >300 | PAK4 | >300 |
| TYRO3 | >300 | PDK1 | >300 |
| | | PIM1 | >300 |
| | | PKACa | >300 |
| | | PKCa | >300 |
| | | PKD2 | >300 |
| | | ROCK1 | >300 |
| | | SGK | >300 |
| | | JNK2 | >300 |
| | | MAP2K1 | >300 |
| | | AMPKa1/b1/g1 | >300 |
| | | RAF1 | >300 |

### Test Example 3

### TCF/Lymphoid Enhancer Factor (LEF) Reporter Gene Assay

Human colorectal cancer cell lines DLD-1 and HCT-116 were obtained from Health Science Research Resources Bank and American Type Culture Collection, respectively. Full length human TNIK inserted into pCIneo-HA vector (Promega) was kindly gifted from Dr. Kenichi Kariya (Ryukyu University). DLD1 and HCT-116 cells were co-transfected in triplicate with canonical (TOP-FLASH) or mutant (FOP-FLASH) TCF/LEF luciferase reporter, phRL-TK (Promega) (an internal standard), and pCIneo-HA-TNIK or pCIneo-HA (control plasmid). Twenty-four hours after transfection, compound I or vehicle were added to the cells at final concentration of 0.078125, 0.15625, 0.3125, and 0.625 μM. After subsequent 24 h, reporter activity was assayed by using the Dual-Luciferase Reporter Assay System (Promega) according to the instruction manuals. The test results are shown in FIG. 1. Results were normalized to Renilla values of each sample. The reporter assay results represent the average and standard deviation of triplicate assays. Compound 1 inhibited β-catenin/TCF4-mediated transcription in DLD1 and HCT-116 in a concentration-dependent manner.

### Example

The following examples are illustrative only, and not intended to limit the scope of the limit the present invention.

Abbreviations and symbols used in the following descriptions mean as follows:
CDCl₃: chloroform-d
D₂O: deuterium oxide
DCM: dichloromethane
DMA: dimethylacetamide
DMF: dimethyl formamide
DMSO: dimethyl sulfoxide
EtOH: ethanol
EtOAc: ethyl acetate
HCl: hydrochloric acid
K₂CO₃: potassium carbonate
MeOH: methanol
MgSO₄: magnesium sulfate
NaHCO₃: sodium bicarbonate
Na₂SO₄: sodium sulfate
NH₄Cl: ammonium chloride
NH₃: ammonia
N₂: nitrogen
POCl₃: phosphorous oxychloride
THF: tetrahydrofuran
TFA: trifluoroacetic acid
Xantphos: 4,5-Bis(diphenylphosphino)-9,9-dimethylxanthene
EDC: 1-ethyl-3-(3-dimethylaminopropyl) carbodiimide hydrochloride
HOBT: hydroxybenzotriazole
min.: minute(s)
h or hr(s): hour(s)
RT or rt: room temperature
sat.: saturated
aq.: aqueous
TLC: thin layer chromatography
HPLC: high performance liquid chromatography
Prep HPLC: preparative HPLC
LCMS: high performance liquid chromatography/mass spectrometry
MS: mass spectrometry
NMR: nuclear magnetic resonance

### Example 1-16, 20, 26, 34, 36, 46, 52 and 54

Each of the Examples shown in the following Table A were synthesized according to the procedure described in the following Example 4 using appropriate starting materials.

TABLE A

| | | LCMS |
|---|---|---|
| Ex. No. | ¹H-NMR δ (ppm) | m/z [M + H]⁺ |
| 1 | (DMSO-d₆): 2.41 (s, 3H), 6.95 (t, 1H, J = 7.6 Hz), 7.30 (dd, | 352.9 |
| | 2H, J = 8.4, 7.6 Hz), 7.43 (d, 2H, J = 8.0 Hz), 7.65-7.75 (m, | |
| | 3H), 7.75-7.9 (m, 3H), 10.06 (s, 1H), 12.59 (s, 1H). | |
| 2 | (DMSO-d₆): 6.95 (t, 1H, J = 7.2 Hz), 7.30 (dd, 2H, J = 8.4, | 356.9 |
| | 7.2 Hz), 7.47 (t, 2H, J = 8.8 Hz), 7.7-7.8 (m, 3H), 7.87 (br, | |
| | 1H), 7.97 (dd, 2H, J = 8.4, 5.2 Hz), 10.09 (s, 1H), 12.63 (s, | |
| | 1H). | |
| 3 | (DMSO-d₆): 6.95 (t, 1H, J = 7.6 Hz), 7.30 (dd, 2H, J = 8.8, | 372.8 |
| | 7.6 Hz), 7.71 (d, 2H, J = 8.8 Hz), 7.72-7.8 (m, 3H), 7.91 (d, | |
| | 2H, J = 8.8 Hz), 10.10 (s, 1H), 12.67 (s, 1H). | |
| 5 | (DMSO-d₆): 2.09 (s, 3H), 7.25 (d, 2H J = 8.6 Hz), 7.7-7.9 | 480.0 |
| | (m, 8H), 10.28 (s, 1H), 10.33 (s, 1H), 12.54 (s, 1H). | |
| 6 | (DMSO-d₆): 2.09 (s, 3H), 2.31 (s, 3H), 6.76 (d, 1H, J = 7.4 | 410.2 |
| | Hz), 7.18 (t, 1H, J = 7.8 Hz), 7.35 (s, 1H), 7.6-7.7 (m, 2H), | |
| | 7.7-7.9 (m, 5H), 9.95 (s, 1H), 10.33 (s, 1H), 12.53 (s, 1H). | |
| 7 | (DMSO-d₆): 2.10 (s, 3H), 7.60 (d, 2H, J = 8.5 Hz), 7.7-7.9 | 464.4 |
| | (m, 6H), 7.95 (d, 2H, J = 8.6 Hz), 10.33 (s, 1H), 10.50 (s, | |
| | 1H), 12.56 (s, 1H). | |
| 8 | (DMSO-d₆): 2.09 (s, 3H), 2.84 (s, 6H), 6.73 (d, 2H, J = 8.4 | 439.0 |
| | Hz), 7.5-7.6 (m, 2H), 7.7-7.9 (m, 4H), 9.65 (s, 1H), 10.32 (s, | |
| | 1H), 12.45 (s, 1H). | |
| 9 | (DMSO-d₆): 2.09 (s, 3H), 2.28 (s, 3H), 6.97 (t, 1H, J = 7.3 | 410.4 |
| | Hz), 7.1-7.2 (m, 2H), 7.42 (s, 1H), 7.7-7.9 (m, 5H), 8.08 (d, | |
| | 1H, J = 8.3 Hz), 9.04 (s, 1H), 10.31 (s, 1H), 12.46 (s, 1H). | |
| 10 | (DMSO-d₆): 2.08 (s, 3H), 2.23 (s, 6H), 7.1-7.2 (m, 4H), | 424.2 |
| | 7.7-7.8 (m, 5H), 8.89 (s, 1H), 10.30 (s, 1H), 12.34 (s, 1H). | |
| 11 | (DMSO-d₆): 2.09 (s, 3H), 3.72 (s, 3H), 6.87 (d, 2H, J = 8.7 | 426.4 |
| | Hz), 7.6-7.7 (m, 3H), 7.7-7.9 (m, 5H), 9.87 (s, 1H), 10.34 (s, | |
| | 1H), 12.51(s, 1H). | |
| 12 | (DMSO-d₆): 6.95 (t, 1H, J = 7.2 Hz), 7.30 (dd, 2H, J = 8.4, | 422.9 |
| | 7.6 Hz), 7.63 (d, 2H, J = 8.0 Hz), 7.7-7.8 (m, 3H), 7.89 (br, | |
| | 1H), 8.03 (d, 2H, J = 8.8 Hz), 10.11 (s, 1H), 12.68 (s, 1H). | |
| 13 | (DMSO-d₆): 2.09 (s, 3H), 7.30 (d, 2H, J = 8.7 Hz), 7.7-7.9 | 428.2 |
| | (m, 8H), 10.22 (s, 1H), 10.34 (s, 1H), 12.55 (s, 1H). | [M − H]⁺ |
| 14 | (DMSO-d₆): 2.09 (s, 3H), 2.23 (s, 3H), 2.25 (s, 3H), 7.00-7.1 | 424.0 |
| | (m, 2H), 7.37 (s, 1H), 7.7-7.9 (m, 6H), 8.99 (s, 1H), 10.33 (s, | |
| | 1H), 12.44 (s, 1H). | |
| 15 | (DMSO-d₆): 2.09 (s, 3H), 2.16 (s, 3H), 2.27 (s, 3H), 6.96 (d, | 424.6 |
| | 1H, J = 7.4 Hz), 7.09 (t, 1H, J = 7.7 Hz), 7.33 (s, 1H), 7.67 | |
| | (d, 1H, J = 7.9 Hz), 7.7-7.9 (m, 5H), 9.08 (s, 1H), 10.34 (s, | |
| | 1H), 12.42 (s, 1H). | |
| 16 | (DMSO-d₆): 3.86 (s, 3H), 6.94 (t, 1H, J = 7.3 Hz), 7.16 (d, | 368.8 |
| | 2H, J = 8.4 Hz), 7.30 (t, 2H, J = 7.6 Hz), 7.68 (br, 1H), 7.73 | |
| | (d, 2H, J = 8.0 Hz), 7.80 (br, 1H), 7.86 (d, 2H, J = 7.7 Hz), | |
| | 10.03 (s, 1H), 12.5 (s, 1H). | |
| 20 | (DMSO-d₆): 2.13 (s, 3H), 7.3-7.4 (m, 1H), 7.7-7.9 (m, 6H), | 395.3 |
| | 8.15 (br, 1H), 8.4-8.5 (m, 1H), 8.70 (br, 1H), 10.30 (s, 1H), | [M − H]⁺ |
| | 10.33 (s, 1H), 12.57 (S, 1H). | |
| 26 | (DMSO-d₆): 3.02 (s, 6H), 3.72 (s, 3H), 6.83 (d, 2H, J = 8.8 | 412.4 |
| | Hz), 6.87 (d, 2H, J = 8.8 Hz), 7.55 (s, 1H), 7.64 (d, 2H, J = | |
| | 8.9 Hz), 7.7-7.8 (m, 3H), 9.79 (s, 1H), 12.33 (s, 1H). | |
| 34 | (DMSO-d₆): 3.73 (s, 3H), 5.22 (s, 2H), 6.88 (d, 2H, J = 8.8 | 474.9 |
| | Hz), 7.24 (d, 2H, 8.8 Hz), 7.3-7.55 (m, 5H), 7.63 (br, 1H), | |
| | 7.66 (d, 2H, J = 9.2 Hz), 7.80 (br, 1H), 7.86 (d, 2H, J = 8.8 | |
| | Hz), 9.86 (s, 1H), 12.50 (s, 1H). | |
| 36 | (DMSO-d₆): 2.35-2.6 (m, 4H), 2.72 (t, 2H, J = 5.6 Hz), | 498.4 |
| | 3.5-3.6 (m, 4H), 3.73 (s, 3H), 4.20 (t, 2H, J = 5.6 Hz), 6.88 | |
| | (d, 2H, J = 8.8 Hz), 7.16 (d, 2H, J = 9.2 Hz), 7.63 (br, 1H), | |
| | 7.66 (d, 2H, J = 9.2 Hz), 7.80 (br, 1H), 7.84 (d, 2H, J = 8.8 | |
| | Hz), 9.86 (s, 1H), 12.49 (s, 1H). | |
| 46 | (DMSO-d₆): 3.72 (s, 3H), 3.86 (s, 3H), 6.88 (d, 2H, J = 8.7 | 399.2 |
| | Hz), 7.15 (d, 2H, J = 8.6 Hz), 7.60 (s, 1H), 7.65 (d, 2H, J = | |
| | 8.6 Hz), 7.78 (s, 1H), 7.85 (d, 2H, J = 8.5 Hz), 9.84 (s, 1H), | |
| | 12.48 (s, 1H). | |
| 52 | NMR (DMSO-d₆, 400 MHz): δ 3.86 (s, 3H), 7.09 (t, 1H, J = | 387.1 |
| | 8.8 Hz), 7.14 (d, 1H, J = 8.76 Hz), 7.71-7.81 (m, 4H), 7.87 | |
| | (d, 1H, J = 8.72 Hz), 10.08 (s, 1H), 12.51 (s, 1H). | |
| 54 | (DMSO-d₆): 1.5-1.7 (m, 2H), 1.85-2.1 (m, 2H), 3.4-3.6 (m, | 468.9 |
| | 2H), 3.73 (s, 3H), 3.8-4.0 (m, 2H), 4.73 (t, 1H, J = 4.0 Hz), | |
| | 6.88 (d, 2H, J = 9.2 Hz), 7.19 (d, 2H, J = 8.8 Hz), 7.62 (br, | |
| | 1H), 7.65 (d, 2H, J = 8.8 Hz), 7.79 (br, 1H), 7.84 (d, 2H, J = | |
| | 8.8 Hz), 9.85 (s, 1H), 12.48 (s, 1H). | |

### Example 4

### 5-(4-acetamidobenzamido)-2-(4-fluorophenylamino)thiazole-4-carboxamide

### (a) 5-amino-2-(4-fluorophenylamino)thiazole-4-carboxamide

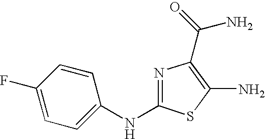

To a suspension of 2-amino-2-cyanoacetamide (0.25 g, 2.5 mmol) in EtOAc (7 mL) was added 4-fluorophenyl isothiocyanate (0.386 g, 2.5 mmol), and the mixture was refluxed for 30 min. The solvent was evaporated and the residue was purified by silica gel column chromatography eluted with 2% MeOH in DCM to give 0.4 g (52% yield) of the titled compound.

¹H-NMR (400 MHz, DMSO-d₆) δ (ppm) 6.65 (s, 2H), 6.89 (br, 2H), 7.04 (t, 2H J=8.7 Hz), 7.6-7.7 (m, 2H), 9.56 (s, 1H). LCMS m/z [M+H]⁺253.0.

### (b) 5-(4-acetamidobenzamido)-2-(4-fluorophenylamino)thiazole-4-carboxamide

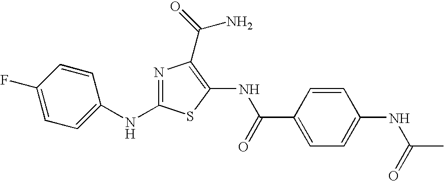

To a mixture of 4-acetamidobenzoic acid (0.106 g, 0.59 mmol) and catalytic amount of DMF in dry THF (5 mL) was added dropwise oxalyl chloride (0.06 mL, 0.79 mmol) at 0° C., and the mixture was stirred for 2 h at rt. The solvent was evaporated, and the residual oxalyl chloride was removed with azeotropic distillation using toluene under nitrogen atmosphere. The resulting acid chloride was then dissolved in pyridine (5 mL) and cooled to 0° C. To this solution, a solution of 5-amino-2-(4-fluorophenylamino)thiazole-4-carboxamide (0.1 g, 0.39 mmol) in pyridine (5 mL) was added at 0° C., and the mixture was stirred for 12 h at rt. The solvent was evaporated, and the residue was suspended into 1M HCl, and the resulting solids were collected. The solids were washed with water (10 mL), ether (20 mL) and dried. The crude solids were purified by silica gel column chromatography eluted with 3% MeOH in DCM to give 38 mg (10% yield) of the titled compound.

¹H-NMR (400 MHz, DMSO-d₆) δ (ppm) 2.09 (s, 3H), 7.01 (t, 2H, J=8.7 Hz), 7.7-7.9 (m, 8H), 10.09 (s, 1H), 10.32 (s, 1H), 12.5 (s, 1H). LCMS m/z [M+H]⁺414.4.

### Example 17

### 5-(4-acetamidobenzamido)-2-(4-hydroxyphenylamino)thiazole-4-carboxamide

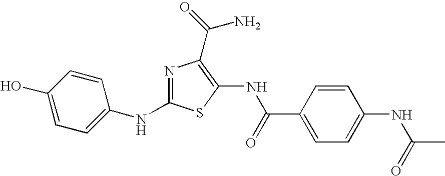

To a solution of 5-(4-acetamidobenzamido)-2-(4-methoxyphenylamino)thiazole-4-carboxamide (100 mg, 0.23 mmol) in 1,2-dichloroethane (10 mL) was added dropwise BBr₃ (587.5 mg, 2.35 mmol) at 0° C. under nitrogen atmosphere, and the mixture was stirred at rt for 5 h. The reaction mixture was quenched with 1M HCl (5 ml), and the organic layer was separated and concentrated. The resulting solids were collected, and washed successively with hexane and ether, and dried to give 70 mg (72% yield) of the titled compound.

¹H-NMR (400 MHz, DMSO-d₆) δ (ppm) 2.09 (s, 3H), 6.71 (d, 2H, J=8.6 Hz), 7.51 (d, 2H, J=8.6 Hz), 7.58 (br, 1H), 7.7-7.9 (m, 5H), 9.71 (s, 1H), 10.33 (s, 1H), 12.49 (s, 1H). LCMS m/z [M+H]⁺412.1.

### Example 18

### tert-butyl-4-[5-(4-acetamidobenzamido)-4-carbamoylthiazol-2-ylamino]phenylcarbamate

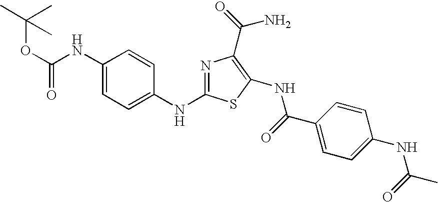

### (a) tert-butyl-4-isothiocyanatophenylcarbamate

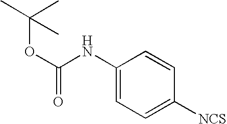

To a mixture of tent-butyl-4-aminophenylcarbamate (0.5 g, 2.4 mmol) and triethylamine (0.98 mL, 7.2 mmol) in THF (45 mL) was added dropwise thiophosgene (0.2 mL, 2.64 mmol) at 0° C., and the mixture was stirred at rt for 30 min. The reaction mixture was quenched with water and extracted with ether (2×30 mL). The organic layer was dried over Na₂SO₄ and concentrated to give 0.5 g (83% yield) of the titled compound, which was used for next step without further purification.

¹H-NMR (400 MHz, CDCl₃) δ (ppm) 1.50 (s, 9H), 6.50 (s, 1H), 7.14 (d, 2H, J=8.6 Hz), 7.34 (d, 2H, J=8.4 Hz). LCMS m/z [M+H]⁺251.2.

### (b) tert-butyl-4-(5-amino-4-carbamoylthiazol-2-ylamino)phenylcarbamate

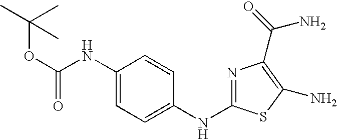

To a suspension of 2-amino-2-cyanoacetamide (0.178 g, 1.8 mmol) in EtOAc (10 mL) was added tert-butyl-4-isothiocyanatophenylcarbamate (0.5 g, 2.0 mmol), and the mixture was refluxed for 30 min. The solvent was evaporated, and the residue was purified by silica gel column chromatography eluted with 2% MeOH in DCM to give 0.4 g (57% yield) of the titled compound.

¹H-NMR (400 MHz, DMSO-d₆) δ (ppm) 1.46 (s, 9H), 6.62 (s, 2H), 6.88 (s, 2H), 7.3-7.4 (m, 2H), 7.46 (d, 2H, J=8.8 Hz), 9.12 (s, 1H), 9.39 (s, 1H). LCMS m/z [M+H]⁺350.3.

### (c) tert-butyl-4-[5-(4-acetamidobenzamido)-4-carbamoylthiazol-2-ylamino]phenylcarbamate

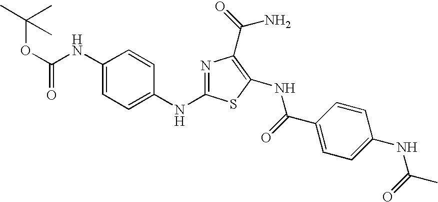

To a mixture of 4-acetamidobenzoic acid (0.256 g, 1.43 mmol) and catalytic amount of THF in dry THF (15 mL) was added dropwise oxalyl chloride (0.25 mL, 2.86 mmol) at 0° C., and the mixture was stirred for 2 h at rt. The solvent was evaporated, and the residual oxalyl chloride was removed with azeotropic distillation using toluene under nitrogen atmosphere. The resulting acid chloride was then dissolved in pyridine (10 mL) and cooled to 0° C. To this solution, a solution of tert-butyl-4-(5-amino-4-carbamoylthiazol-2-ylamino)phenylcarbamate (0.4 g, 1.14 mmol) in pyridine (5 mL) was added at 0° C., and the mixture was stirred for 12 h at rt. The solvent was evaporated, and the residue was suspended into 1M HCl, and the resulting solids were collected. The solids were washed with water (10 mL), ether (20 mL) and dried. The crude solids were purified by silica gel column chromatography eluted with 2-5% MeOH in DCM to give 125 mg (21% yield) of the titled compound.

¹H-NMR (400 MHz, DMSO-d₆) δ (ppm) 1.47 (s, 9H), 2.09 (s, 3H), 7.3-7.4 (m, 2H), 7.62 (d, 2H, J=8.5 Hz), 7.72 (s, 1H), 7.8-7.9 (m, 5H), 9.20 (br, 1H), 9.92 (s, 1H), 10.33 (s, 1H), 12.54 (s, 1H). LCMS m/z [M+H]⁺511.3.

### Example 19

### 5-(4-acetamidobenzamido)-2-(4-aminophenylamino)thiazole-4-carboxamide

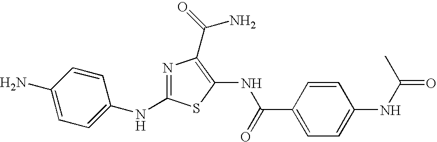

tert-butyl-4-[5-(4-acetamidobenzamido)-4-carbamoylthiazol-2-ylamino]phenylcarbamate (0.10 g, 19 mmol) was dissolved in 4M HCl in 1,4-dioxane (10 mL) at 0° C. under argon atmosphere, and the mixture was stirred at 0° C. for 3 h. The solvent was evaporated, and the residual acid was removed with azeotropic distillation using toluene. The resulting solids were dried to give 78 mg (98% yield) of the titled compound.

¹H-NMR (400 MHz, DMSO-d₆) δ (ppm) 2.07 (s, 3H), 7.29 (d, 2H, J=8.7 Hz), 7.7-7.9 (m, 8H), 9.93 (br, 2H), 10.36 (s, 1H), 10.38 (s, 1H), 12.56 (s, 1H). LCMS m/z [M+H]⁺411.1.

### Example 21

### 5-(4-fluorobenzamido)-2-(4-methoxyphenylamino)thiazole-4-carboxamide

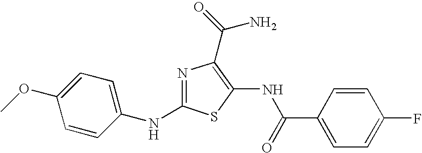

### (a) 5-amino-2-(4-methoxyphenylamino)thiazole-4-carboxamide

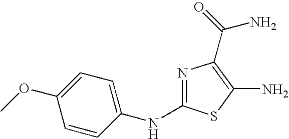

A mixture of 4-methoxyphenylisothiocyanate (1.91 g, 11.53 mmol) and 2-amino-2-cyanoacetamide (1.20 g, 12.11 mmol) in EtOAc (16 mL) was heated at 80° C. for 50 min. The reaction mixture was cooled to rt, and the resulting solids were filtered off. The filtrate was concentrated, and the residue was purified by silica gel chromatography (eluent: 2% MeOH in CHCl₃ to 5% MeOH in CHCl₃) to afford the titled compound (2.63 g, 86% yield).

¹H-NMR (400 MHz, DMSO-d₆) δ (ppm) 3.70 (s, 3H), 6.61 (s, 2H), 6.7-7.0 (m, 4H), 7.51 (d, 2H, J=9.2 Hz), 9.34 (s, 1H).

### (b) 5-(4-fluorobenzamido)-2-(4-methoxyphenylamino)thiazole-4-carboxamide

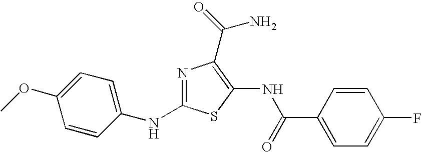

To a solution of 5-amino-2-(4-methoxyphenylamino)thiazole-4-carboxamide (920 mg, 3.48 mmol) in pyridine (15 mL) was added 4-fluorobenzoyl chloride (0.411 mL, 3.48 mmol) at 0° C., the mixture was allowed to warm to rt and stirred for 1 h at rt. The reaction mixture was diluted with EtOAc, and the organic layer was washed successively with water (×2) and brine. The organic layer was dried over Na₂SO₄ and evaporated. The resulting solids were collected and washed with 50% hexane in EtOAc to afford the titled compound (1.04 g, 77% yield).

¹H-NMR (400 MHz, DMSO-d₆) δ (ppm) 3.73 (s, 3H), 6.88 (d, 2H, J=9.2 Hz), 7.46 (t, 2H, J=8.8 Hz), 7.6-7.75 (m, 3H), 7.84 (br, 1H), 7.96 (dd, 2H, J=8.8, 5.2 Hz), 9.89 (s, 1H), 12.58 (s, 1H). LCMS m/z [M+H]⁺386.9.

### Example 22

### 2-(4-methoxyphenylamino)-5-[4-(4-methylpiperazin-1-yl)benzamido]thiazole-4-carboxamide

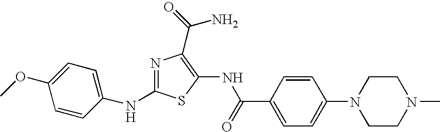

A mixture of 5-(4-fluorobenzamido)-2-(4-methoxyphenylamino)thiazole-4-carboxamide (50 mg, 0.13 mmol) and 1-methylpiperazine (0.072 mL, 0.65 mmol) in N-methylpyrrolidone (0.6 mL) was treated using a microwave synthesizer for 40 min (CEM corp, 180° C.). The reaction mixture was concentrated and the residue was purified by silica gel chromatography (eluent: CHCl₃ to 12% MeOH in CHCl₃) to afford the titled compound (38 mg, 63% yield).

¹H-NMR (400 MHz, DMSO-d₆) δ (ppm) 2.23 (s, 3H), 2.3-2.7 (m, 4H), 3.2-3.5 (m, 4H), 3.72 (s, 3H), 6.88 (d, 2H, J=9.2 Hz), 7.08 (d, 2H, J=9.2 Hz), 7.58 (br, 1H), 7.65 (d, 2H, J=9.2 Hz), 7.73 (d, 2H, J=8.8 Hz), 7.76 (br, 1H), 9.82 (s, 1H), 12.39 (s, 1H). LCMS m/z [M+H]⁺467.0.

### Example 23-24, 27-32, 43, 49 and 55-56

The compounds shown in the following Table B were prepared by following the procedure described for above Example 22 using appropriate starting materials.

TABLE B

| | | LCMS |
|---|---|---|
| Ex. No. | ¹H-NMR δ (ppm) | m/z [M + H]⁺ |
| 23 | (DMSO-d₆): 3.1-3.3 (m, 2H), 3.57 (q, 2H, J = 5.6 Hz), 3.72 | 426.0 |
| | (s, 3H), 4.76 (t, 1H, J = 5.6 Hz), 6.57 (t, 1H, J = 5.6 Hz), | [M − H]⁺ |
| | 6.71 (d, 2H, J = 8.8 Hz), 6.87 (d, 2H, J = 9.2 Hz), 7.54 (br, | |
| | 1H), 7.6-7.7 (m, 4H), 7.72 (br, 1H), 9.79 (s, 1H), 12.28 (s, 1H). | |
| 24 | (DMSO-d₆): 2.19 (s, 6H), 2.45 (t, 2H, J = 6.4 Hz), 3.19 (q, | 455.0 |
| | 2H, J = 6.4 Hz), 3.72 (s, 3H), 6.43 (t, 1H, J = 5.2 Hz), 6.72 | |
| | (d, 2H, J = 8.8 Hz), 6.88 (d, 2H, J = 8.8 Hz), 7.54 (br, 1H), | |
| | 7.6-7.7 (m, 4H), 7.72 (br, 1H), 9.79 (s, 1H), 12.28 (s, 1H). | |
| 27 | (DMSO-d₆): 2.3-2.6 (m, 6H), 3.23 (q, 2H, J = 6.4 Hz), | 497.0 |
| | 3.5-3.7 (m, 4H), 3.72 (s, 3H), 6.46 (t, 1H, J = 5.6 Hz), 6.71 | |
| | (d, 2H, J = 9.2 Hz), 6.88 (d, 2H, J = 9.2 Hz), 7.54 (br, 1H), | |
| | 7.6-7.7 (m, 4H), 7.72 (br, 1H), 9.79 (s, 1H), 12.28 (s, 1H). | |
| 28 | (DMSO-d₆): 3.2-3.4 (m, 4H), 3.6-3.8 (m, 4H), 6.88 (d, 2H, J = | 454.0 |
| | 9.2 Hz), 7.10 (d, 2H, J = 9.2 Hz), 7.59 (br, 1H), 7.65 (d, | |
| | 2H, J = 9.2 Hz), 7.7-7.8 (m, 3H), 9.83 (s, 1H), 12.41 (s, 1H). | |
| 29 | (DMSO-d₆): 1.6-1.8 (m, 4H), 2.4-2.8 (m, 6H), 3.15-3.4 (m, | 481.4 |
| | 2H), 3.72 (s, 3H), 6.52 (t, 1H, J = 5.2 Hz), 6.71 (d, 2H, J = | |
| | 8.8 Hz), 6.88 (d, 2H, J = 9.2 Hz), 7.54 (br, 1H), 7.6-7.7 (m, | |
| | 4H), 7.72 (br, 1H), 9.79 (s, 1H), 12.28 (s, 1H). | |
| 30 | (DMSO-d₆): 2.2-2.8 (m, 10H), 3.54 (q, 2H, J = 6.0 Hz), 3.72 | 497.0 |
| | (s, 3H), 4.45 (t, 1H, J = 5.6 Hz), 6.88 (d, 2H, J = 9.2 Hz), | |
| | 7.08 (d, 2H, J = 9.2 Hz), 7.58 (br, 1H), 7.65 (d, 2H, J = 9.2 | |
| | Hz), 7.73 (d, 2H, J = 9.2 Hz), 7.76 (br, 1H), 9.82 (s, 1H), | |
| | 12.39 (s, 1H). | |
| 31 | (DMSO-d₆): 3.2-3.4 (m, 5H), 3.50 (t, 2H, J = 5.6 Hz), 3.72 | 441.9 |
| | (s, 3H), 6.63 (t, 1H, J = 5.6 Hz), 6.72 (d, 2H, J = 8.8 Hz), | |
| | 6.88 (d, 2H, J = 8.8 Hz), 7.54 (br, 1H), 7.6-7.7 (m, 4H), 7.72 | |
| | (br, 1H), 9.79 (s, 1H), 12.28 (s, 1H). | |
| 32 | (DMSO-d₆): 2.6-2.8 (m, 2H), 3.0-3.5 (m, 4H), 3.72 (s, 3H), | 426.9 |
| | 6.58 (br, 1H), 6.69 (d, 2H, J = 8.4 Hz), 6.87 (d, 2H, J = 9.2 | |
| | Hz), 7.4-7.9 (m, 6H), 9.79 (br, 1H), 12.26 (br, 1H). | |
| 43 | (DMSO-d₆): 1.1-1.3 (m, 2H), 1.5-1.8 (m, 3H), 2.75-2.95 (m, | 482.0 |
| | 2H), 3.28 (t, 2H, J = 5.6 Hz), 3.38 (s, 3H), 3.9-4.1 (m, 2H), | |
| | 4.49 (t, 2H, J = 5.6 Hz), 6.88 (d, 2H, J = 9.2 Hz), 7.06 (d, | |
| | 2H, J = 9.2 Hz), 7.58 (br, 1H), 7.65 (d, 2H, J = 8.8 Hz), 7.71 | |
| | (d, 2H, J = 8.8 Hz), 7.53 (br, 1H), 9.82 (s, 1H), 12.36 (s, 1H). | |
| 49 | (DMSO-d₆): 1.2-1.7 (m, 6H), 2.2-2.7 (m, 6H), 3.1-3.5 (m, | 495.4 |
| | 2H), 3.72 (s, 3H), 6.44 (br, 1H), 6.71 (d, 2H, J = 8.4 Hz), | |
| | 6.87 (d, 2H, J = 9.2 Hz), 7.56 (br, 1H), 7.6-7.7 (br, 1H), | |
| | 7.6-7.7 (m, 4H), 7.73 (br, 1H), 9.80 (s, 1H), 12.29 (s, 1H). | |
| 55 | (DMSO-d₆): 2.89 (dd, 2H, J = 7.6, 6.8 Hz), 3.3-3.5 (m, 2H), | 489.4 |
| | 3.72 (s, 3H), 6.65-6.8 (m, 3H), 6.87 (d, 2H, J = 9.2 Hz), 7.32 | |
| | (d, 2H, J = 6.0 Hz), 7.55 (br, 1H), 7.6-7.7 (m, 4H), 7.72 (br, | |
| | 1H), 8.48 (d, 2H, J = 6.0 Hz), 9.79 (s, 1H), 12.29 (s, 1H). | |
| 56 | (DMSO-d₆): 2.12 (s, 3H), 2.67 (dd, 1H, J = 7.6, 6.4 Hz), | 457.9 |
| | 3.2-3.5 (m, 2H), 3.72 (s, 3H), 6.65-6.8 (m, 3H), 6.87 (d, 2H, | |
| | J = 9.2 Hz), 7.56 (br, 1H), 7.6-7.7 (m, 4H), 7.34 (br, 1H), | |
| | 9.81 (s, 1H), 12.30 (s, 1H). | |

### Example 25

### 5-(4-acetamidobenzamido)-2-(4-acetamidophenylamino)thiazole-4-carboxamide

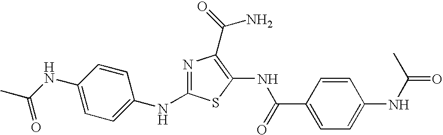

To a solution of 5-(4-acetamidobenzamido)-2-(4-aminophenylamino)thiazole-4-carboxamide (50 mg, 0.12 mmol) in pyridine (2 mL) was added acetyl chloride (14.4 mg, 0.182 mmol) at 0° C. under nitrogen atmosphere, and the mixture was stirred at rt for 2 h. The reaction mixture was concentrated, and the residue was suspended into 1M HCl. The solids were collected by filtration and washed successively with hexane, methanol, and dried to give 52 mg (76% yield) of the titled compound.

¹H-NMR (400 MHz, DMSO-d₆) δ (ppm) 2.01 (s, 3H), 2.09 (s, 3H), 7.51 (d, 2H, J=8.4 Hz), 7.66 (d, 2H, J=8.4 Hz), 7.73 (s, 1H), 7.8-7.9 (m, 5H), 9.81 (s, 1H), 9.98 (s, 1H), 10.34 (s, 1H), 12.54 (s, 1H). LCMS m/z [M+H]⁺453.4.

### Example 33

### 5-(4-aminobenzamido)-2-(4-methoxyphenylamino)thiazole-4-carboxamide

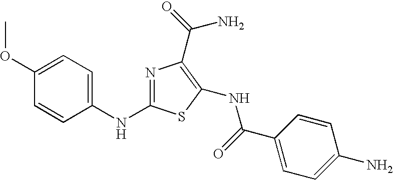

### (a) 2-(4-methoxyphenylamino)-5-(4-nitrobenzamido)thiazole-4-carboxamide

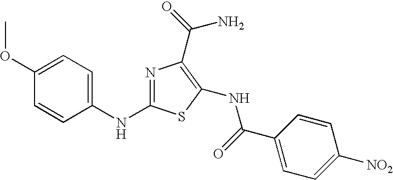

To a solution of 4-nitrobenzoyl chloride (0.7 g, 3.78 mmol) in pyridine (6 mL) was added a solution of 5-amino-2-(4-methoxyphenylamino)thiazole-4-carboxamide (1 g, 3.7 mmol) in pyridine (6 mL) at 0° C., and the mixture was stirred at rt for 12 h. The solvent was evaporated, and the residue was diluted with water and extracted with ethyl acetate. The organic layer was washed successively with 1M HCl (2×100 mL), water (2×50 mL), sat. NaHCO₃ (2×50 mL), water (50 mL) and brine (50 mL). The organic layer was dried over Na₂SO₄ and concentrated to give 0.41 g (27% yield) of the title compound, which was used for next step without further purification.

¹H-NMR (400 MHz, DMSO-d₆) δ (ppm) 3.70 (s, 3H), 6.88 (d, 2H, J=8.7 Hz), 7.66 (d, 2H, J=8.7 Hz), 7.70 (s, 1H), 7.88 (s, 1H), 8.13 (d, 2H, J=8.4 Hz), 8.43 (d, 2H, J=8.4 Hz), 9.93 (s, 1H), 12.76 (s, 1H). LCMS m/z [M+H]⁺414.2.

### (b) 5-(4-aminobenzamido)-2-(4-methoxyphenylamino)thiazole-4-carboxamide

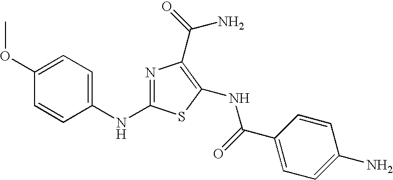

To a solution of 2-(4-methoxyphenylamino)-5-(4-nitrobenzamido)thiazole-4-carboxamide (0.1 g, 0.24 mmol) in THF-EtOH (1:1, 30 mL) was added stannous chloride dihydrate (0.27 g, 1.2 mmol) at rt, and the mixture was refluxed for 5 h. The reaction mixture was concentrated, and the residue was diluted with EtOAc. 1M NaOH was added to the solution until the solution was basic (pH=8-9). The organic layer was separated, and the aqueous layer was extracted with EtOAc (2×50 mL). The combined organic layers were filtered through a bed of Celite. The filtrate was washed with water, and dried over Na₂SO₄ and concentrated. The resulting solids were collected and washed with hexane to give 39 mg (42% yield) of the titled compound.

¹H-NMR (400 MHz, DMSO-d₆) δ (ppm) 3.72 (s, 3H), 6.03 (s, 2H), 6.65 (d, 2H, J=8.3 Hz), 6.87 (d, 2H, J=8.6 Hz), 7.53 (s, 1H), 7.58 (d, 2H, J=8.3 Hz), 7.54 (d, 2H, J=8.5 Hz), 7.70 (s, 1H), 9.78 (s, 1H), 12.25 (s, 1H). LCMS m/z [M+H]⁺384.0.

### Example 35

### 5-(4-hydroxybenzamido)-2-(4-methoxyphenylamino)thiazole-4-carboxamide

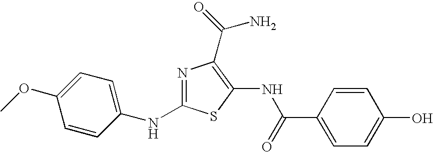

### (a) 4-[4-carbamoyl-2-(4-methoxyphenylamino)thiazol-5-ylcarbamoyl]phenyl acetate

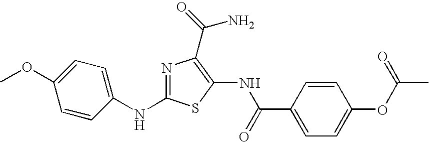

To a suspension of acetoxybenzoic acid (350 mg, 1.97 mmol) in CH₂Cl₂ (36 mL) was added oxalyl chloride (0.696 mL, 7.95 mmol) and catalytic amount of DMF at 0° C., and the mixture was stirred for 5 hr at rt. The solvent was evaporated, and the residual oxalyl chloride was removed with azeotropic distillation using toluene under nitrogen atmosphere. The resulting acid chloride was added to a solution of 5-amino-2-(4-methoxyphenylamino)thiazole-4-carboxamide (350 mg, 1.32 mmol) in pyridine (10 mL) at 0° C., and the mixture was stirred for 2 hr at rt. The reaction mixture was quenched by adding of ice-water, and extracted with EtOAc. The organic layer was washed with water twice, dried over Na₂SO₄ and concentrated. The residue was triturated with 50% EtOAc in Et₂O, and the resulting solids were collected by filtration and washed with 50% EtOAc in Et₂O to afford the titled compound (382 mg, 68% yield).

¹H-NMR (400 MHz, DMSO-d₆) δ (ppm) 2.32 (s, 3H), 3.73 (s, 3H), 6.89 (d, 2H, J=8.8 Hz), 7.39 (d, 2H, J=8.8 Hz), 7.6-7.7 (m, 3H), 7.84 (br, 1H), 7.94 (d, 2H, J=8.8 Hz), 9.90 (s, 1H), 12.59 (s, 1H).

### (b) 5-(4-hydroxybenzamido)-2-(4-methoxyphenylamino)thiazole-4-carboxamide

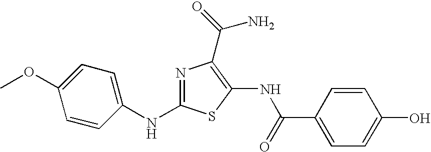

To a suspension of 4-[4-carbamoyl-2-(4-methoxyphenylamino)thiazol-5-ylcarbamoyl]phenyl acetate (350 mg, 0.821 mmol) in dry MeOH (45 mL) was added K₂CO₃ (113 mg, 0.821 mmol), and the mixture was stirred at 50° C. for 20 min. The reaction mixture was cooled to 0° C., and then diluted with water. The solution was acidified with 2M HCl (0.5 mL). The resulting solids were collected by filtration and washed with water to afford the titled compound (250 mg, 79% yield).

¹H-NMR (400 MHz, DMSO-d₆) δ (ppm) 3.72 (s, 3H), 6.88 (d, 2H, J=9.2 Hz), 6.95 (d, 2H, J=8.8 Hz), 7.60 (br, 1H), 7.65 (d, 2H, J=9.2 Hz), 7.7-7.8 (m, 3H), 9.84 (s, 1H), 10.40 (br, 1H), 12.42 (s, 1H). LCMS m/z [M+H]⁺384.8.

### Example 37

### 5-[4-(2-hydroxyacetamido)benzamido]-2-(4-methoxyphenylamino)thiazole-4-carboxamide

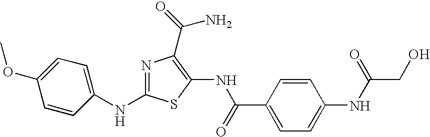

To a mixture of 5-(4-aminobenzamido)-2-(4-methoxyphenylamino)thiazole-4-carboxamide (0.05 g, 0.13 mmol) and triethylamine (0.018 mL) in THF (10 mL) was added dropwise acetoxy acetylchloride (0.016 mL, 0.16 mmol) at 0° C., and the mixture was stirred at rt for 2 h. The reaction mixture was diluted with water and extracted with EtOAc (3×20 mL). The combined organic layers were dried over Na₂SO₄ and concentrated. The resulting solids were dissolved in MeOH (5 mL), and K₂CO₃ (30 mg, 0.22 mmol) and catalytic amount of water was added to this solution. The mixture was stirred at rt for 1 h. The solvent was evaporated, and the residue was purified by silica gel column chromatography eluted with 2% MeOH in DCM to give 7 mg (9% yield) of the titled compound.

¹H-NMR (400 MHz, DMSO-d₆) δ (ppm) 3.72 (s, 3H), 4.04 (d, 2H, J=5.8 Hz), 5.7-5.8 (m, 1H), 6.88 (d, 2H, J=8.6 Hz), 7.6-7.7 (m, 3H), 7.80 (s, 1H), 7.85 (d, 2H, J=8.4 Hz), 7.94 (d, 2H, J=8.4 Hz), 9.86 (s, 1H), 10.07 (s, 1H), 12.51 (s, 1H). LCMS m/z [M+H]⁺442.3.

### Example 38

### 5-{4-[2-(dimethylamino)acetamido]benzamido}-2-(4-methoxyphenylamino)thiazole-4-carboxamide

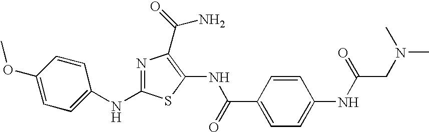

### (a) 5-[4-(2-bromoacetamido)benzamido]-2-(4-methoxyphenylamino)thiazole-4-carboxamide

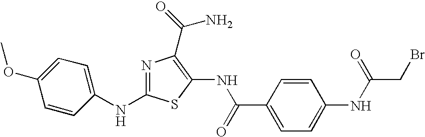

To a mixture of 5-(4-aminobenzamido)-2-(4-methoxyphenylamino)thiazole-4-carboxamide (0.3 g, 0.78 mmol) and triethylamine (0.213 mL, 1.56 mmol) in THF (30 mL) was added dropwise bromoacetylchloride (135 mg, 0.86 mmol) at 0° C., and the mixture was stirred at rt for 4 h. The reaction mixture was diluted with water and extracted with EtOAc. The organic layer was dried over Na₂SO₄ and concentrated. The resulting solids were washed with MeOH, and dried to give 0.3 g (75% yield) of the titled compound.

¹H-NMR (400 MHz, DMSO-d₆) δ (ppm) 3.72 (s, 3H), 4.09 (s, 2H), 6.88 (d, 2H, J=8.6 Hz), 7.6-7.7 (m, 3H), 7.8-7.9 (m, 5H), 9.86 (s, 1H), 10.75 (s, 1H), 12.52 (s, 1H). LCMS m/z [M+H]⁺506.2.

### (b) 5-{4-[2-(dimethylamino)acetamido]benzamido}-2-(4-methoxyphenylamino)thiazole-4-carboxamide

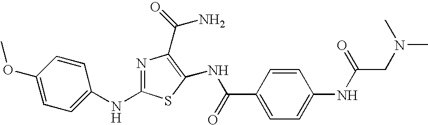

To a solution of dimethylamine (11% in MeOH, 0.07 mL, 0.138 mmol) in THF (10 mL) was added NaHCO₃ (11.5 mg, 0.138 mmol), and the mixture was stirred for 15 min at rt.

A solution of 5-[4-(2-bromoacetamido)benzamido]-2-(4-methoxyphenylamino)thiazole-4-carboxamide (70 mg, 0.138 mmol) in THF was added slowly to this solution at 0° C., and the mixture was stirred at rt overnight. The solvent was evaporated, and water (10 mL) was added to the residue. The resulting solids were collected by filtration and purified by silica gel column chromatography eluted with 60% EtOAc in hexanes to give 15 mg (23% yield) of the titled compound.

¹H-NMR (400 MHz, DMSO-d₆) δ (ppm) 2.28 (s, 6H), 3.12 (s, 2H), 3.72 (s, 3H), 6.88 (d, 2H, J=8.6 Hz), 7.6-7.7 (m, 3H), 7.80 (s, 1H), 7.83 (d, 2H, J=8.5 Hz), 7.90 (d, 2H, J=8.4 Hz), 9.86 (s, 1H), 10.11 (s, 1H), 12.51 (s, 1H). LCMS m/z [M+H]⁺469.3.

### Example 39-42

The compounds shown in the following Table C were prepared by following the procedure described for above Example 38 using appropriate starting materials.

TABLE C

| | | LCMS |
|---|---|---|
| Ex. No. | ¹H-NMR δ (ppm) | m/z [M + H]⁺ |
| 39 | (DMSO-d₆): 1.7-1.8 (m, 4H), 2.5-2.7 (m, 4H), 3.3-3.4 (m, | 495.1 |
| | 2H), 3.73 (s, 3H), 6.8-6.9 (m, 2H), 7.6-7.7 (m, 3H), 7.8-7.9 | |
| | (m, 5H), 9.86 (s, 1H), 10.10 (s, 1H), 12.51 (s, 1H). | |
| 40 | (DMSO-d₆): 3.18 (s, 2H), 3.2-3.4 (m, 4H), 3.6-3.6 (m, 4H), | 511.5 |
| | 3.72 (s, 3H), 6.88 (d, 2H, J = 8.8 Hz), 7.62 (s, 1H), 7.65 (d, | |
| | 2H, J = 8.7 Hz), 7.8-7.9 (m, 5H), 9.85 (s, 1H), 10.12 (s, 1H), | |
| | 12.51 (s, 1H). | |
| 41 | (DMSO-d₆): 1.4-1.5 (m, 2H), 1.5-1.6 (m, 4H), 2.4-2.5 (m, | 509.4 |
| | 4H), 3.11 (s, 2H), 3.72 (s, 3H), 6.87 (d, 2H, J = 8.8 Hz), | |
| | 7.6-7.7 (m, 3H), 7.8-7.9 (m, 5H), 9.85 (s, 1H), 10.04 (s, 1H), | |
| | 12.51 (s, 1H). | |
| 42 | (CD₃OD): 2.31 (s, 3H), 2.5-2.7 (m, 8H), 3.22 (s, 2H), 3.77 | 524.2 |
| | (s, 3H), 6.90 (d, 2H, J = 8.7 Hz), 7.51 (d, 2H, J = 8.8 Hz), | |
| | 7.81 (d, 2H, J = 8.6 Hz), 7.92 (d, 2H, J = 8.4 Hz). | |

### Example 44

### 2-(4-methoxyphenylamino)-5-{4-[(4-methylpiperazin-1-yl)methyl]benzamido}thiazole-4-carboxamide

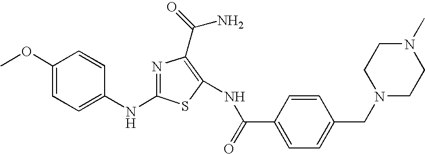

To a mixture of 5-amino-2-(4-methoxyphenylamino)thiazole-4-carboxamide (50 mg, 0.189 mmol) and N,N-diisopropylethylamine (27 mg, 0.208 mmol) in DMA (2 mL) was added 4-chloromethylbenzoyl chloride (39 mg, 0.208 mmol) at 0° C., and the mixture was stirred at rt. After 2 h, 1-methylpiperazine (95 mg, 0.946 mmol) was added to this mixture, and the stirring was continued for 3 h at rt. The reaction mixture was diluted with EtOAc, and the organic layer was washed with water twice, dried over Na₂SO₄ and concentrated. The residue was purified with silica gel chromatography (eluent: CHCl₃ to 12% MeOH in CHCl₃) to afford the titled compound (34 mg, 37% yield).

¹H-NMR (400 MHz, DMSO-d₆) δ (ppm) 2.17 (s, 3H), 2.2-2.6 (m, 8H), 3.55 (s, 2H), 3.73 (s, 3H), 6.88 (d, 2H, J=9.2 Hz), 7.53 (d, 2H, J=8.0 Hz), 7.6-7.7 (m, 3H), 7.83 (br, 1H), 7.85 (d, 2H, J=8.4 Hz), 9.88 (s, 1H), 12.56 (s, 1H), LCMS m/z [M+H]⁺481.4.

### Example 45

### 2-(4-methoxyphenylamino)-5-{4-[2-(pyrrolidin-1-yl)ethoxy]benzamido}thiazole-4-carboxamide

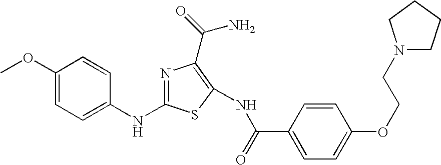

To a mixture of 5-(4-hydroxybenzamido)-2-(4-methoxyphenylamino)thiazole-4-carboxamide (50 mg, 013 mmol) and potassium carbonate (47 mg, 0.34 mmol) in DMF (2 mL) was added 2-chloroethylpyrrolidine hydrochloride (29 mg, 0.17 mmol) at rt, and the mixture was stirred at 80° C. for 2 hr. The reaction mixture was diluted with EtOAc, and washed with water twice, dried over Na₂SO₄ and concentrated. The residue was purified by silica gel chromatography (eluent: CHCl₃ to 12% MeOH in CHCl₃) to afford the titled compound (16 mg, 26% yield).

¹H-NMR (400 MHz, DMSO-d₆) δ (ppm) 1.5-1.8 (m, 4H), 2.3-2.6 (m, 4H), 2.7-3.0 (m, 2H), 3.73 (s, 3H), 4.18 (t, 2H, J=5.6 Hz), 6.88 (d, 2H, J=9.2 Hz), 7.17 (d, 2H, J=8.8 Hz), 7.63 (br, 1H), 7.66 (d, 2H, J=8.8 Hz), 7.80 (br, 1H), 7.84 (d, 2H, J=8.8 Hz), 9.86 (s, 1H), 12.49 (s, 1H), LCMS m/z [M+H]⁺482.4.

### Example 47

### 5-(4-methoxybenzamido)-2-(pyridin-4-ylamino)thiazole-4-carboxamide

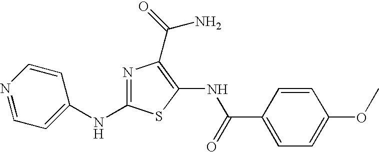

### (a) ethyl 5-amino-2-bromothiazole-4-carboxylate

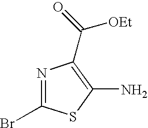

N-Bromosuccinimide (0.54 g, 3.03 mmol) was added to a solution of 5-aminothiazole-4-carboxylic acid ethyl ester (0.44 g, 2.53 mmol), prepared according to the procedure described by Golankiewicz et al. (Tetrahedron, 41 (24), 5989-5994 (1985)) in acetonitrile (10 mL), and the mixture was stirred for 30 min. The reaction mixture was diluted with EtOAc (50 mL) and washed with 5% K₂CO₃ aq. solution (25 mL) followed by brine (25 mL). The organic layer was dried over Na₂SO₄ and concentrated. The residue was purified by silica gel column chromatography eluted with 15% EtOAc in hexane to give 0.37 g (58% yield) of the titled compound.

¹H-NMR (400 MHz, CDCl₃) δ (ppm) 1.38 (t, 3H, J=7.1 Hz), 4.37 (q, 2H, J=7.1 Hz), 6.02 (s, 2H). LCMS m/z [M+H]⁺253.1.

### (b) ethyl 2-bromo-5-(4-methoxybenzamido)thiazole-4-carboxylate

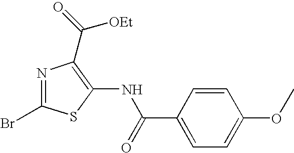

To a solution of p-anisoyl chloride (1.87 g, 11 mmol) in pyridine (30 mL) was added dropwise a solution of ethyl 5-amino-2-bromothiazole-4-carboxylate (1.5 g, 5.5 mmol) in pyridine (52 mL) over a period of 10 min at 0° C., the mixture was stirred for 72 h at rt. The reaction mixture was diluted with 1M HCl and extracted with EtOAc (4×200 mL). The combined organic extracts were washed with brine, dried over Na₂SO₄ and concentrated. The residue was purified by silica gel column chromatography eluted with 10% EtOAc in hexane to give 1.35 g (58% yield) of the titled compound.

¹H-NMR (400 MHz, CDCl₃) δ (ppm) 1.45 (t, 3H, J=7.1 Hz), 3.89 (s, 3H), 4.48 (q, 2H, J=7.1 Hz), 7.02 (d, 2H, J=8.6 Hz), 7.96 (d, 2H, J=8.6 Hz), 11.72 (s, 1H). LCMS m/z [M+H]⁺385.2.

### (c) ethyl 5-(4-methoxybenzamido)-2-(pyridin-4-ylamino)thiazole-4-carboxylate

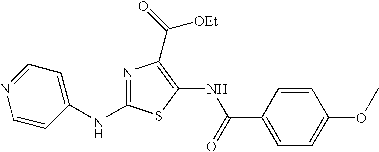

To the solution of ethyl 2-bromo-5-(4-methoxybenzamido)thiazole-4-carboxylate (0.2 g, 0.519 mmol) in 1,4-dioxane (18 mL) was added Xantphos (0.060 g, 0.1 mmol) and Pd₂(dba)₃ (0.047 g, 0.05 mmol) under argon gas. Cesium carbonate (0.337 g, 1.03 mmol) and 4-aminopyridine (0.048 g, 0.519 mmol) was then added to this solution, and the mixture was refluxed for 5 h. The reaction mixture was filtered through a bed of Celite, and the celite was washed with EtOAc (3×5 mL). The filtrate was concentrated, and the residue was purified by silica gel column chromatography eluted with 40% EtOAc in Hexane to give 54 mg (26% yield) of the titled compound.

¹H-NMR (400 MHz, DMSO-d₆) δ (ppm) 1.38 (t, 3H, J=7.2 Hz), 3.87 (s, 3H), 4.42 (q, 2H, J=7.0 Hz), 7.17 (d, 2H, J=8.7 Hz), 7.58 (d, 2H, J=5.6 Hz), 7.92 (d, 2H, J=8.6 Hz), 8.39 (d, 2H, J=5.2 Hz), 10.60 (s, 1H), 11.39 (s, 1H). LCMS m/z [M+H]⁺399.1.

### (d) 5-(4-methoxybenzamido)-2-(pyridin-4-ylamino)thiazole-4-carboxamide

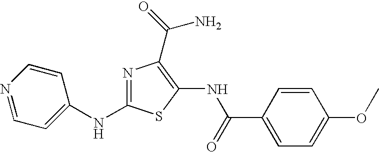

To a solution of ethyl 5-(4-methoxybenzamido)-2-(pyridin-4-ylamino)thiazole-4-carboxylate (0.05 g, 0.12 mmol) in THF (3 mL) was added 7M NH₃ in MeOH (7 mL), and the solution was heated at 80° C. in a sealed tube for 5 h. The solvent was evaporated, and the resulting solid was collected. The solids were washed with ether and dried to give 21 mg (45% yield) of the titled compound.

¹H-NMR (400 MHz, DMSO-d₆) δ (ppm) 3.86 (s, 3H), 7.16 (d, 2H, J=8.3 Hz), 7.6-7.8 (m, 2H), 7.8-8.0 (m, 4H), 8.3-8.4 (m, 2H), 10.50 (s, 1H), 12.58 (s, 1H). LCMS m/z [M+H]⁺370.4.

### Example 50-51 and 57

The compounds shown in the following Table D were prepared by following the procedure described for above Example 47 using appropriate starting materials.

TABLE D

| | | LCMS |
|---|---|---|
| Ex. No. | ¹H-NMR δ (ppm) | m/z [M + H]⁺ |
| 50 | NMR (DMSO-d6, 400 MHz): δ 3.86 (s, 3H), 7.16 (d, 2H, J = | 370.3 |
| | 8.28 Hz), 7.31 (dd, 1H, J = 4.68 and 7.8 Hz), 7.8-7.9 (m, | |
| | 4H), 8.15 (d, 1H, J = 3.92 Hz), 8.47 (d, 1H, J = 8.0 Hz), 8.68 | |
| | (s, 1H), 10.29 (s, 1H), 12.56 (s, 1H). | |
| 51 | NMR (DMSO-d6, 400 MHz): δ 3.86 (s, 3H), 7.16 (d, 2H, J = | 414.1 |
| | 8.4 Hz), 7.38 (m, 1H), 7.8-8.0 (m, 4H), 7.98 (d, 2H, J = 9.0 | |
| | Hz), 8.17 (d, 2H, J = 8.9 Hz), 10.88 (s, 1H), 12.58 (s, 1H). | |
| 57 | (DMSO-d₆): δ 3.86 (s, 3H), 7.1-7.3 (m, 4H), 7.6-8.0 (m, | 446.3 |
| | 8H), 10.47 (s, 1H), 12.58 (s, 1H). | [M − H]⁺ |

### Example 48

### 2-(4-methoxyphenylamino)-5-{4-[N-(methylsulfonyl)methylsulfonamido]benzamido}thiazole-4-carboxamide

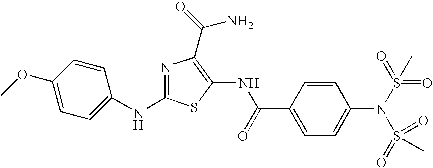

To a mixture of 5-(4-aminobenzamido)-2-(4-methoxyphenylamino)thiazole-4-carboxamide (0.150 g, 0.3916 mmol) and Et₃N (0.2 mL, 1.56 mmol) in THF (10 mL) was added dropwise methanesulfonyl chloride (0.09 mL, 1.174 mmol) at 0° C., and the mixture was stirred at rt for 2 h. The reaction mixture was concentrated. Water was added to the residual oil, and the resulting solids were collected by filtration to give 100 mg (47% yield) of the titled compound.

¹H-NMR (400 MHz, DMSO-d₆) δ (ppm) 3.59 (s, 6H), 3.72 (s, 3H), 6.88 (d, 2H, J=8.8 Hz), 7.6-7.7 (m, 3H), 7.78 (d, 2H, J=8.1 Hz), 7.86 (br, 1H), 7.97 (d, 2H, J=8.1 Hz), 9.91 (s, 1H), 12.63 (s, 1H). LCMS m/z [M+H]⁺540.4.

### Example 53

### 2-(4-methoxyphenylamino)-5-[4-(methylsulfonamido)benzamido]thiazole-4-carboxamide

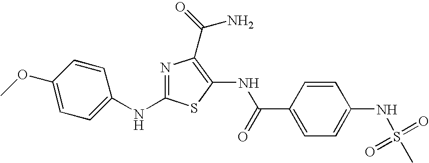

To a solution of 5-(4-aminobenzamido)-2-(4-methoxyphenylamino)thiazole-4-carboxamide (0.06 g, 0.156 mmol) in THF (10 mL) was added dropwise freshly distilled methanesulfonyl chloride (0.02 mL, 0.313 mmol) at 0° C., then Et₃N (0.06 mL, 0.468 mmol) was added to this solution at 0° C. The mixture was stirred at rt for 12 h. To complete the reaction, another 0.5 mol equivalent of methanesulfonyl chloride and 1 mol equivalent Et₃N was added to the mixture, and the stirring was continued overnight. The reaction mixture was concentrated, and the residue was purified by silica gel column chromatography eluted with 50% ethyl acetate in hexanes to give 11.5 mg (15% yield) of the titled compound.

¹H-NMR (400 MHz, DMSO-d₆) δ (ppm) 33.12 (s, 3H), 3.72 (s, 3H), 6.87 (d, 2H, J=8.9 Hz), 7.36 (d, 2H, J=8.6 Hz), 7.64 (br, 2H), 7.65 (d, 4H, J=9.0 Hz), 7.82 (br, 1H), 7.85 (t, 2H, J=8.6 Hz), 9.88 (s, 1H), 12.50 (s, 1H). LCMS m/z [M+H]⁺462.2.

### Example 58

### Preparation of Tablets

Tablets each containing 100 mg of 5-(4-acetamidobenzamido)-2-(phenylamino)thiazole-4-carboxamide (Compound 1) are obtained by the following procedure.

### Formulation:

| Ingredients | Amount |
|---|---|
| Compound 1 | 100 parts by weight |
| Cornstarch | 46 parts by weight |
| Microcrystalline cellulose | 98 parts by weight |
| Hydroxypropyl cellulose | 2 parts by weight |
| Magnesium stearate | 4 parts by weight |

Procedure:

Compound 1, cornstarch and microcrystalline cellulose are mixed and the mixture is added to hydroxypropyl cellulose dissolved in 50 parts by weight of water, followed by sufficient kneading. The kneaded mixture is passed through a sieve to granulate, dried mixed with magnesium stearate and then compressed into tablets of 250 mg each.

### Example 59

### Preparation of Granules

Granules containing 5-(4-acetamidobenzamido)-2-(phenylamino) thiazole-4-carboxamide (Compound 1) are obtained by the following procedure.

### Formulation:

| Ingredients | Amount |
|---|---|
| Compound 1 | 200 parts by weight |
| Lactose | 185 parts by weight |
| Cornstarch | 109 parts by weight |
| Hydroxypropyl cellulose | 6 parts by weight |

Procedure:

Compound 1, lactose and cornstarch are mixed and the mixture is added to hydroxypropyl cellulose dissolved in 120 parts by weight of water, followed by sufficient kneading. The kneaded mixture is passed through a 20 mesh sieve to granulate, dried and then size-adjusted to obtain granules containing 200 mg of Compound 1 per 500 mg of granule.

### Example 60

### Preparation of Capsules

Capsules each containing 100 mg of 5-(4-acetamidobenzamido)-2-(phenylamino)thiazole-4-carboxamide (Compound 1) are obtained by the following procedure.

### Formulation:

| Ingredients | Amount |
|---|---|
| Compound 1 | 100 parts by weight |
| Lactose | 35 parts by weight |
| Cornstarch | 60 parts by weight |
| Magnesium stearate | 5 parts by weight |

Procedure:

Compound 1, lactose, cornstarch and magnesium stearate are well mixed and 200 mg each of the powder mixture is encapsulated to obtain capsules.

1. A TNIK inhibitor comprising a compound represented by the general formula (I) or a pharmaceutically acceptable salt thereof as an active ingredient: 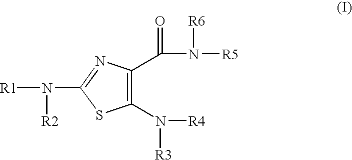
Wherein R1, R2, R3, R4, R5 and R6 represent independently a hydrogen atom or a substituent group.

2. A method for the treatment of cancer patients with TNIK inhibitor, which comprises
administering an effective amount of the compound or pharmaceutically acceptable salt thereof according to claim 1 as an active ingredient.

3. The method of claim 2, wherein the cancer is solid cancer.

4. The method of claim 2 or 3, wherein the cancer is colorectal cancer.

5. The method of claim 2 or 3, wherein the cancer is pancreatic cancer.

6. The method of claim 2 or 3, wherein the cancer is non-small cell lung cancer.

7. The method of claim 2 or 3, wherein the cancer is prostate cancer.

8. The method of claim 2 or 3, wherein the cancer is breast cancer.

9. The method of claim 2 or 3, wherein the TNIK inhibitor is administered orally.

10. The method of claim 2 or 3, wherein the TNIK inhibitor is administered intravenously.

11. A compound represented by the following general formula: 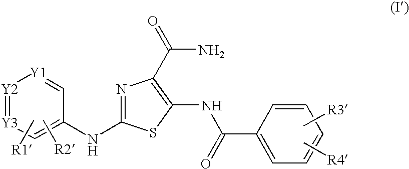
(wherein R1′ and R2′ independently represent a hydrogen atom, a halogen atom, a hydroxy group, a substituted or unsubstituted alkyl group, a substituted or unsubstituted alkoxy group, a substituted or unsubstituted amino group, an acylamino group, a nitro group, a substituted or unsubstituted alkoxycarbonylamino group, R3′ and R4′ independently represent a hydrogen atom, a halogen atom, a hydroxy group, a substituted or unsubstituted alkyl group, a substituted or unsubstituted alkoxy group, a substituted or unsubstituted amino group, a substituted or unsubstituted acylamino group or a substituted or unsubstituted alkyl sulfonamido group, and Y1, Y2 and Y3 independently represent a nitrogen atom or a carbon atom.), or a pharmaceutically acceptable salt thereof.
